SEQUENCE LISTING (18 sequences)

SEQ ID NO: 1 - DNA, 6474 bp, Artificial Sequence
  PKS18-PputidaOpt
atggacgtgctgatcttcggcgaccagaccgccgatcagtacccgctgctgcgcaaggcctgcacctggaagtcgaacgcgaccctgaccaccttcctggaccgcatcagcgtggtgatccgcgaggaagtgcagaagctgccacgcacccagcgcgatcagatcccgaacttcctgaccacctgggacctggtggaagcctactacgccaagggcctgaagatcccggaaatcgagagctgcatggtgaccatcgcgcagctgagccactacatcggctacttcgccgagtcgccgaccgagctgccgaacccgagcaacacccgtgtggtgggcctgtgcaccggtctgctggccggtagcgtggtggccagcgcgcgtagcctgagcgaactgctgccactggccaccgaagccgtgcgcgtggccttccgtgccggtacctgcgtgggtgccgccaaagaggccctggaacagagcagcgccagcaaggacagctggtccaccatcgtgaccaacatcagcgaggatgccgccaaggacgcgatcgccgccttccacgaggaacagaagatccccaccctggcgcaggcctacatcagcgccgtgagcaccatggcgctgaccatcagcggtccgccagccaccaccaagcgcctgctggaaaaggacgccttcaagtcgagcgcccgtgtgccgatcccggtgtatgccccataccacgcctcgcacctgtacgcccaggccgacatcgaccgcatcctggacaaggatgccatccgccatctgcagcaattccgtccggtggcgctggtgcatagcgccgccaccggtaagtgccagaccgcgaccaacaccctggaactggtccgcaccgcgctgcacgagatgctggtcgaaccggtgcgctgggacagcctgctgagcgaggtggtgagccaggtgacctcggcgccaaacgcgcagtgcagcgtgagcgccttcggcgtgacctcgatcaccaacagcctggcgagcgccctgaagaacggtggtcagagcgccatcaccgtgcgcgaccaaagcgcctgggtgccagccgaacacgacagccgtggtcgcacccagaacgacaagatcgccatcgtgggcatgagcggtcgcttcccaggtgcggcgaacccggaagcgctgtgggatctgctggaacgtggcctggacgtgcaccgcgaagtcccagccgatcgcttcgacgccaaggcgcattgcgacccgagcggcaagggcaagaacaagtcgcacaccccgtacggctgcttcatcgacgaaccgggtctgttcgacccacgcttcttcaacatgagcccacgcgaagccgcgcagaccgatccgatgggtcgtctggccctgaccaccgcctacgaagccctggaaatgagcggctacgtgccgaaccgcaccccgagcaccaagctggaacgcatcggcaccttctacggccagacctcggacgactggcgtgagatcaacgccgccgagaacatcgacacctacttcatcaccggtggtgtgcgtgccttcgcgccaggccgtatcaactactacttcaagttctcgggtccgagctacagcgtggacaccgcctgcagcagcagcctggccgccatccagctggcgtgcaccagcctgtgggccggtgattgcgataccgcctgcgccggtggcctgaacgtcctgaccaacccggacatcttcagcggtctgagcaagggccagttcctgagcaagaccggcagctgcaagacctacgacaacgacgccgacggctactgccgtggcgacggttgcggctcggtggtgctgaagcgctacgaagatgcgatcgcggacaaggacaacatcctgggctgcatcctgggtgccgcgaccaaccatagcgccgaggccgtgagcatcacccatccgcatgccggtgcgcaagagtacctgtacaacaaggtgctgagcaacgccggtgtggacgcccacgaaatcagctacgtggaaatgcacggcaccggcacccaagccggtgacggcatcgagatgacctcggtgaccaacgcgttcgccccacgccatcgtcagcgcaccccagaacaaaccctgcacctgggtgccatcaaggccaacatcggccatggcgaagcggccagcggcatcaactcgctggtgaaggtcctgatgatgatgaagaaaaacgcgatcccagccaacgtcggcatcaagggcgtgatgaacaagaccttcccgaaggatctggcccagcgcaacgtgcacatcgaaaccacccaggtcgcctggccacgcaagggtgccgagaagcgcaagatcttcctgaacaacttcagcgcggccggtggcaacaccgccgtgatcctggaagatggtccgctgccggaagaaccgaagggcgtcgatccgcgtaccatgcacatggtcaccgtgtcggcccgttcgatcgccagcctgaagaagaacatcaacaacctgatcgacttcgtggacgagaacccgtcgatcaccctgccgagcctggcctacaccaccaccgcgcgtcgcatccagcacaactaccgcgtcgccttctgcgtgtcggacatgagcaaggtgaaggacggcctgcgtgcccagctgaaggatacctactcgccactgccgatggtgccgaccaagaccgcgttcaccttcaccggtcagggcagccagtataccggtctgggtcagaagctgtacgaggacctggaaaccttcaagaacgacatcgatcagctggacaagctggcccgtctgcacagcctgccgtcgatcctgccactgctgaccggtgccgatgtggcgaccctgtcgccagtggtcatccaactgggcatggcctgcatccaggtggccctggcgcgtatgtggggtgcctggggtgtccgtccgatcgccgtcatcggtcacagcctgggcgaatatgcggccctgcacgtggccggtgtgatcagcgcctcggacatggtgctgctggtcggtcgccgtgcgcaaatcctggaagaggaatgcaccgcgaacacccacggcatgctggccgtcaagggcagcgtggaagccatcaaagccgcgctgggcgacaagatgaccgagatcgcctgcgtgaacggtccggaagaaaccgtgctgtgcggcaccgtggacgtggtggacagcaccaacgagctgctggcctcgaaaggcttcaagagcaccaaactgaacgtgccgttcgccttccattcggcccaggtggaacccatcctggaaaagttcaaggccgtggccgccagcgtgaccttcaacaagccggtcgtgccggtgatgtcgccactgaacggcgatatcatcgtcgaagccggtatcatcggtccggactatctggcgcgtcatgcccgtgaaaccgtgaacttctgcaccgcgctgaccaacggccaagagcagaagctgttcgacgcgaaaaccgcctggctggaagtgggtgcccatccggtgtgcagcggcatggtgaagtcgagcctgggtggctccccagtggccgccggttcgctgcgtcgtaacgaggacccgtggaaaaccctgtcgaacaccctgaccaccctgtacctggccggtgtgtacatcgacttcaacgagtatcaccgcctgttcaacgacgcgcaccagatgtacaccctgccaacctacgccttcgacagcaagaagtactggctggactaccacaacaactggaccctgaccaagggcgaagtgctgcaagccgcgccagccgtgtcgaccatcgaagcggcgccagtggaagaagccccaagcaagctgtcgaccacctcgtgccacaagatcgtgcgcgaggatctgcacgccaactcgggcaccgtcgtggtgcagagcgacctgagcgacccgaagctgaaggccaccatcaccggtcaccaggtgaacggcaccccactgaccccgtcgtcgctgtatgccgaccaggccatgaccgtggccgactacctgtaccagcagctgcgtccaggcaccgaaaccccaggtctgaacgtgtgcagcatggaagtcaccaagaccctgatcccgcagtacccaccgccagcgaccggtcaacatctgcaaatcgagggcaacgccgacctggaagccagccaggtcaagatcaccttccgcaccgtgagcgcggacggcagcaagatcctggccgaacatgcggtgggcatcgtgaaatacgaggacgtgaacgcctggaaagaagagtggggtcgtatccagtacatggtgcagtcgcagatcgacatgctgcaacagaaactggcgaccggtgcggcccacaaggtcctgcgtggcatggcgtacaagctgttcaaggcgctggtcacctacgccgataactatcgcggtatggaagaggtcatcctggatggcaagcagaccgaggccaccgcctcggtgcagttccaaaccaccgaggccgacggcgagttcctgtgcagcccctactggatcgactcgctggcccacctgagcggcttcatcgtgaacgcgagcgaccatctggacagcgagaacagcgtgtacatcagccacggctggggcagcatcaagatcgcgggtaagctgagcccagagaagaaataccgctcgtacgtgcgcatgcagccagcgccaggcaacatctcggtgggcgacgtctacatcatggacggcaccgagatcatcggcatggtcatgggcctgaaattccagaacatcccacgtcgtgccctgaacatcatgatgccgccagcgggtaaagccgccagctcgccagccgcgaaagcggtggcccgtccagcgccaaaggcgctgccagcggcggccccagtgaaaacccaagcgccagtgccgaaggccgtcaaagccccaaagccagtgaaggccgtgaaggtcgcggcgccagccggtgtgacctccaaggtgatgaacatcgtggcccaagagatcgacgtggacatgtcggaactggtggacgaagccgccttcgagaacctgggcgtcgactccctgctgtccctgaccatctcggcgcgtttccgcgaagaactggacatggacatcccgtcgaccctgttcaccgactgctcgaccgtgggcgagctgaagaagcacttcagccagttcgacggtgcgaccatcgtcgaggacgactcgagcgtcacctccgacgaacccagcggtgccccaaccccgttcgaagccccagcgggtcagtcggatagcaccccagccagctcggccggttcggacgacggccatgacgacgtgaagccgagcgcgccaaccgaaggtggtgcctcgctggcgcgtaaactggtggcggaagagatgggtgtcgacgtgagcgagatcaccgacgacctggacctgaccgacatcggtatggatagcctgatgagcctgaccatcctgggcagcatgcgcgaagccaccggtcgtgacctgccagccgatttcctgaccgtgaacgtcaccatcaaggatatcgaaaccgcgctgggcatgcgtccgcagccgaaagcggccaagccagcggccaaagcctcgagcaaagcgccacagctgagcgaagtgaacaagaagctggcgtccctgccggacgtgagcaacctgccgccagccagcagcgtcctgctgcaaggcaacccaaaaaccgcgaccaagaaattcttcctggtgccggatggcagcggctcggccacctcgtacatctcgatcccaaacatcagcccgagcatggccgtgtacggcctgaactgcccgttcatgaagtgcccagaaaagtggacctgcggtgtcgagggcgtgagccgtctgtacctgaacgagatcaagcgtcgtcagccgagcggtccgtacctgatcggtggttggagcgccggtggtgtcatggcgtacgaagtggcccagcaactggtcaacgcgggtgagaaggtggaaagcctggtgctgatcgatgccccatgcccagtcgcgctggatcccctgccagcgcgtctgcatatcttcttcgaccagatcggcctgctgggcaccggtaaaccaggtggcaccccaagctggctgctgccgcatttcgcgagcgcgatccagaacctgaaggactacgacccgaccccgatggacccgaagatcgccccaccagtgctggccatctggtgcaccgacggcgtgtgcccaaacccagaggatccgcgtccaccaccaggcgaaggcgaggacccagcgccaatgaagtggctgctgaacaaccgcaccgacttcagcgacaacggttgggcccagctgctgcccaaagagaacttccagtacgccgtgatgggtggcaaccatttcaccatgatgaagggcgaccatggcgtcaccctgggcaagctgatccagcagggtctgaagctgtaataa

SEQ ID NO: 2 - DNA, 807 bp, Artificial Sequence
  SEQ ID NO 2 (Brn1-PputidaOpt)
atggccaacatcgagcagacctggtcgctggccggtaaggtggccgtggtgaccggtagcggtcgtggcatcggcaaggccatggcgatcgagctggccaaacgcggtgccaaggtcgccgtgaactacgccaacgccgtggaaggtgccgaacaggtggtgaaagagatcaaggccctgggcaacggcagcgacgcccacgccttcaaagccaacgtgggcaacgtggaagaaagcgagaagctgatggacgacgtggtgaagcacttcggcaagctggacatctgctgctcgaacagcggtgtggtgagcttcggccacttcaaggacgtgaccccagaggaattcgaccgcgtgttcaacatcaacacccgtggccagttcttcgtggccaaagccgcctacaagcgcatggaaatgggtggccgtatcatcctgatgggcagcatcaccggtcaggccaagggcgtgccgaagcacgccgtgtacagcggcagcaagggtgccatcgaaaccttcacccgctgcatggccgtcgatgccggtgagaagaaggtgaccgtcaactgcgtggccccaggcggtatcaagaccgacatgtaccacgccgtctgccgtgagtacatcccgaacggcgaccagctgagcgacgaccaggtggacgagtacgcctgcacctggtcgccacacaaccgtgtgggtcagccgatcgatatcgcccgtgtggtctgcttcctggccagccaggacggcgactgggtgaacggcaaggtgatcggcatcgatggtgccgcctgcatgtaataa

SEQ ID NO: 3 - DNA, 810 bp, Artificial Sequence
  SEQ ID NO 3 (Brn2-PputidaOpt)
atggtggtgaccgtgccgagcgccagcctggaaggtcaggtggccctgatcaccggtgccggtcgtggcatcggtcgcggttgcgccatcgagctgggcaaacgtggtgccagcgtggtggtgaactacgtgagcagcgaaggcccagccaacgaggtgtgcgagatcatccgtggcttcaacaacggtgccaaggccgtggccatccaggccgacgtgagcaaggtgagcgaaatccaccgcctgttcgaggaagccaagcaggccttcggcaagatcaacatcgtgatgagcaacagcggcaccgagagctgggacaagaccgaggaaatcagcgaagagaagtacgaccacgtgttcaacctgaacacccgtgcgcagttcttcgtgggccagaccgcctacaagtacatcgaggacaacggtcgcatcatcctgatgtcgtcgatcgccgccggtctgctgggcgtgaaggaccacgcgctgtacaacgccagcaagatggccgtgatcggcttcatcaaggccttcgccaccgatttcggcaagcgtggcgtgaccgtgaacggcgtggccccaggcggtatcaagagcgacatgttcacccagaacgcctggcactacatcccaggtggcaccccagagtggccagccgagaagatcgagagcctgatggcctcgcactgcccactgggtcgttgcgccgtgccggaagatgtggcccgtgtggtggccttcctggccagcgaggatggcggttgggtgaacggccaggtgctgaccatcagcggtggcagcagccagtaataa

SEQ ID NO: 4 - DNA, 561 bp, Artificial Sequence
  SEQ ID NO 4 (Scd1-PputidaOpt)
atgttcgagaagaaagagctgcagccgaccttcgaggaagtgatgggctgccagagcgcctgctacgagtgggccgacagctacgacagcaaggactgggaccgcctgcgcaagtgcgtggccccaaccctgaagatcgactaccgcagcttcctggacaagatgtgggaagccatgccagccgacgagttcgtggccatggcgagcgatccagccgtgctgggtaacccgctgctgaaaacccagcacttcatcggtggcacccgctgggaaaagaccgccgaggacgagatcaccggctaccatcagctgcgcgtgccgcaccagcgctacaccgacgaaagccgtaccaccgtggccgtgaagggccacgcgcacagcttcaacacccactggtacaagaaaatcgacggcgagtggaagttcgccggtctgaacccggacatccgctggtacgagtacgacttcgacaaggtgttcgccgagggtcgcgagcagctgggcgaagccaaagccgccgcgggtatcccagaaaccgcgccaggccaggccgtgtaataa

SEQ ID NO: 5 - DNA, 1095 bp, Artificial Sequence
  SEQ ID NO 5 (CHS1-PputidaOpt)
atggcgaccctgtgccgtccggaagtggccgtgccggaattcatcatcacccgtgaggacaccctgaagctggccgagcgcctgcatgcgcgtaacccgcagctgaaactggtgctgcgcctgatcaacggcaccggtgtgcagaagcgccacatcgtccagccgctggaacaggccctggcgcacccaggcttcgagatccgcaaccgcatctacgagcgcgaggccaagcgtcgcgtgccaccggtgatccgtcaggcgctggaaaacgccgagctgaccgcgcgtgacatcgacgccatcatcttcgtgagctgcaccggcttcatgatgcccagcatgaccgcctatctgatcaacgccatgggcttccgccacgacacccgtcagatcccgatggcgcaactgggttgcgccgccggcgcggccgccatctcgcgtgcccacgacttctgcgccgcgcacgccgaagccaacgtgctgatcgtggcctgcgagttctgcagcctgtgctaccagccgaccgacgacgacgtgggcagcctgctgagcgacggcctgttcggtgacgccgtgggtgccgtggtggtgcgtggcagcggtggcaccggcatccgcctggaacgcaacgaggcgtatctgatcccggacaccgaggaatggatctcgtacgccgtcaaggataccggcttccacttccgtctggacaagcgcgtgccaggcaccatgaaaccgctggcgccagtgatgcgtcgcctggtggccagccatggttgggacgtcggcaacctggacttctacatcgtgcatgccggtggtccgcgtatcctgaacgacctgtgcaagttcctggaagtgccgcaagaggccttccgctacagccgtgcgaccctgaccgagtacggcaacatcgccagcgccgtggtgctggacagcctgggtcgtctgttcgccgaccgtccgatgccggatggtgcccgtggcgtgatcgccggtttcggtccaggcatcaccgccgagatggccgtcggcagctgggtgaacgaccacggccaaagccgtggcggtgcccgtagcagcagcgccgcctaataa

SEQ ID NO: 6 - DNA, 966 bp, Artificial Sequence
  SEQ ID NO 6 (BdsA-PputidaOpt)
atgaccaacgccagcctgaaaaccccgagcctgcgcttcaccctgggtggcgatcgtccggtgtgccgcatgggctacggtgccatgtacctgaccggtccaggctactggggtccgccagccgatccggatgccgccgtgcgcctgctgcgtcgtgccgtggacctgggcgtgaacttcatcgacaccgccgacgcctacggtccggacaccaacgagcagatcatccgtcgtgccctgcatccgtatccagccggtctggtcatcaccaccaaaggcggtctgctgcgctcgggtcccgaggactggaccgtgcagggtcgtccgtacatcgtcccgtgcggtcgcccagcctacctgcgccagcaggtcgaaatgtcgctgcgcaacctgggcctggaacgcatcgagctgtaccagctgcaccgcatcgacccagccgtgccgctggccgatcagctgggcgaactggcccgtctgaaagagcagggcaagatccaccacatcggcctgagcggtcagccggaagtgaccgtggaacagctggccaaagcgcgtgagatcaccgagatcgccgccgtggaaaacctgtacaacgtggccgaccgcagcggtgagaaggccctgcgctacgccgaacagcacggcatggcgttcatcccgtggttcccgatgggccacggcgacctggccaaaccgggtagcgtgctgtccggtctggcgaaagaacgcggtgcgaccccagcgcaactggccctggcctggctgctgcatcgtagcccaagcatcctggcgatcccaggcaccagcagcatcaagcacctggaagagaactgccgtgcggccgacatgaagctggacgagtcggagctggccgccatcgaagatgccgcggccgccgccaagacctggcgtccgagcgccagcgaatcgcgtgccgcgcacaccaaagaaaccccgtaataa

SEQ ID NO: 7 - DNA, 336 bp, Artificial Sequence
  SEQ ID NO 7 (BdsB-PputidaOpt)
atggtgctgcataccgcgcaccgcgagctgtgccgtgatcgtgccgccggtcgtgtggtgggcaaagccgcggcggcgacccatgagcgcgaactgatccgtatgaccggtggcaccctgcgcatggacgtgcagcacatcttcgccaacgaccacttcggtgccgccttcggcgtgctgcgtgcccatctgggtggtgccagcctggccatgccgttctgcggtgtgtggcgcttccgtggcggtctgatcaccgagcactgggagaacgcctacgacgtggccagcgtgatggccttcctgggcgacgaaagcggtgccccaccggcctaataa

SEQ ID NO: 8 - DNA, 1122 bp, Artificial Sequence
  SEQ ID NO 8 (RppA-PputidaOpt)
atggcgaccctgtgccgtccagcgatcgccgtgccggaacacgtgatcaccatgcagcagaccctggacctggcgcgtgaaacccatgccggtcatccgcagcgcgacctggtgctgcgcctgatccagaacaccggtgtgcagacccgtcacctggtgcagccgatcgaaaagaccctggcgcacccaggcttcgaggtgcgcaaccaggtgtacgaggccgaggcgaaaacccgtgtgccggaagtggtgcgtcgtgccctggccaacgccgaaaccgagccgagcgagatcgacctgatcgtgtacgtgagctgcaccggcttcatgatgccgagcctgaccgcctggatcatcaacagcatgggcttccgtccggaaacccgtcagctgccgatcgcgcaactgggctgcgccgccggtggtgccgccatcaaccgtgcgcatgacttctgcgtggcgtacccggacagcaacgtgctgatcgtgagctgcgagttctgcagcctgtgctaccagccgaccgacatcggcgtgggcagcctgctgagcaacggcctgttcggtgatgccctgtcggccgccgtggtccgtggccaaggtggcaccggtatgcgcctggaacgcaacggcagccatctggtgccggacaccgaggactggatcagctacgccgtgcgcgataccggcttccacttccaactggacaagcgcgtgccaggcaccatggaaatgctggccccagtgctgctggatctggtggacctgcacggctggtcggtgccgaacatggatttcttcatcgtgcatgccggtggtccacgcatcctggacgacctgtgccacttcctggatctgccaccggaaatgttccgctactcgcgtgcgaccctgaccgagcgtggcaacatcgccagcagcgtggtgttcgacgcgctggcccgtctgttcgacgatggcggtgccgccgaaagcgcccaaggtctgatcgccggtttcggtccgggtatcaccgccgaagtggccgtcggctcgtgggccaaagaaggtctgggcgccgacgtgggtcgcgatctggacgagctggaactgaccgccggtgtggccctgagcggttaataa

SEQ ID NO: 9 - DNA, 6474 bp, Artificial Sequence
  SEQ ID NO 9 (PKS18-EcoliOpt)
atggatgtgctgatttttggtgatcagaccgcagatcagtatccgctgctgcgtaaagcatgtacctggaaaagcaatgcaaccctgaccacctttctggatcgtattagcgttgttattcgtgaagaagttcagaaactgcctcgtacacagcgtgatcagattccgaattttctgaccacatgggatttagtggaagcctattatgcaaaaggtctgaaaatcccggaaatcgaaagctgtatggttaccattgcacagctgagccattatatcggttattttgcagaaagcccgaccgaactgccgaatccgagcaatacccgtgttgttggtctgtgtaccggtctgctggcaggtagcgttgtggcaagcgcacgtagcctgagcgaactgctgccgctggcaaccgaagcagttcgtgttgcatttcgtgcaggcacctgtgttggtgcagcaaaagaagcactggaacagagcagcgcaagcaaagatagctggtcaaccattgttaccaacattagcgaagatgcagccaaagatgcaattgccgcatttcatgaagaacagaaaataccgacactggcacaggcatatattagcgcagttagcaccatggcactgaccattagcggtccgcctgcaaccaccaaacgtctgctggaaaaagatgcatttaaaagcagcgcacgtgttccgattccggtttatgcaccgtatcatgcaagccatctgtatgcacaggccgatattgatcgtattctggataaagacgcaattcgtcatctgcagcagtttcgtccggttgcactggttcatagcgcagcaaccggtaaatgtcagaccgccaccaatacactggaactggttcgtaccgcactgcatgagatgctggttgaaccggtgcgttgggatagcctgctgagcgaagttgttagccaggttaccagcgcaccgaatgcacagtgtagcgttagcgcatttggtgtgaccagcattaccaatagcctggccagcgcactgaaaaatggtggtcagagcgcaattaccgttcgtgatcagtcagcctgggttcctgcagaacatgatagccgtggtcgtacccagaatgataaaattgccattgttggtatgagcggtcgttttccgggtgcagccaatccggaagcactgtgggacctgctggaacgtggtctggatgttcatcgtgaagttccggcagatcgttttgatgcaaaagcacattgtgatccgagcggtaaaggtaaaaacaaaagtcataccccgtatggctgctttattgatgaaccgggtctgtttgatccgcgttttttcaatatgagtccgcgtgaagcagcacagaccgatccgatgggtcgtctggcgctgaccaccgcatatgaagccctggaaatgagcggttatgttccgaatcgtaccccgagcaccaaactggaacgtattggcaccttttatggtcagaccagtgatgattggcgtgaaattaatgcagccgaaaacatcgatacctattttatcaccggtggtgttcgtgcatttgcaccgggtcgtattaactattatttcaaattttccggtccgagctatagcgttgataccgcatgtagcagcagcctggcagcaattcagctggcatgtaccagcctgtgggcaggcgattgtgataccgcctgtgccggtggtctgaatgtgctgacaaatccggatatctttagcggtctgagcaaaggtcagtttctgtcaaaaaccggtagctgcaaaacctatgataatgatgcagatggttattgccgtggtgatggttgtggtagcgtggttctgaaacgttatgaagatgccattgcggacaaagataatattctgggctgtattctgggtgccgcaaccaatcatagtgcagaagccgttagcattacccatccgcatgccggtgcacaagaatatctgtataacaaagttctgagcaatgccggtgttgatgcacatgaaattagctatgttgaaatgcatggtacaggcacccaagccggtgatggtattgaaatgaccagcgtgaccaatgcgtttgcaccgcgtcatcgtcagcgtacaccggaacagaccctgcatctgggtgcaattaaagcaaatattggtcatggtgaagccgcaagcggtattaatagtctggttaaagtgctgatgatgatgaaaaagaatgccattccggcaaacgtgggtattaaaggtgttatgaataagacctttccgaaagatctggcacagcgtaatgttcatatcgaaaccacacaggttgcatggcctcgtaaaggtgcagaaaaacgtaaaatcttcctgaacaattttagcgcagccggtggtaataccgcagttattctggaagatggtccgctgccggaagaaccgaaaggtgtggatccgcgtacaatgcatatggtgaccgttagtgcacgtagcattgcaagcctgaaaaaaaacattaacaacctgatcgatttcgtggatgaaaacccgagtattaccctgccgagcctggcatataccaccaccgcacgtcgtattcagcataattatcgtgttgccttttgcgttagcgacatgagcaaagttaaagatggtctgcgtgcccagctgaaagatacctatagtcctctgccgatggttccgaccaaaaccgcatttacgtttaccggtcagggtagccagtatacaggtctgggccagaaactgtatgaagatctggaaaccttcaaaaacgacattgatcagctggataaactggcacgtctgcatagcctgccgagtattctgccgctgctgaccggtgcagatgttgcgaccctgagtccggttgtgattcagttaggtatggcatgtattcaggttgccctggcacgtatgtggggtgcatggggtgttcgtccgattgccgtgattggtcatagcctgggtgaatatgcagccctgcatgttgcgggtgttattagcgccagcgatatggttctgctggttggtcgtcgtgcacagatcctggaagaagaatgtaccgcaaatacccatggtatgctggcagttaaaggtagtgttgaagccattaaagcagcactgggcgataaaatgaccgaaattgcatgtgttaatggtccggaagaaaccgttctgtgtggcaccgttgatgttgttgatagcaccaatgaactgctggcaagtaaaggctttaaatcgaccaaactgaatgtcccgtttgcatttcattcagcacaggtggaaccgattctggaaaaattcaaagcagttgcagcaagcgtgacctttaacaaaccggttgttccggttatgagccctctgaatggtgatattattgttgaagcgggtattatcggtccggattatctggcacgccatgcacgtgaaaccgtgaatttttgtacagccctgaccaatggtcaagagcaaaaactgtttgatgcgaaaaccgcctggctggaagttggtgcacatccggtttgtagcggtatggtgaaaagcagcttaggtggttcaccggttgcagcaggtagcctgcgtcgtaatgaagatccgtggaaaaccctgtcaaataccctgacaaccctgtatctggcaggcgtttatattgattttaacgaatatcaccgcctgttcaatgatgcccatcagatgtatacactgccgacctatgcatttgacagcaaaaaatactggctggattatcacaataattggaccctgacgaaaggtgaagtgctgcaggcagcaccggcagtgagtaccattgaagcggcaccggttgaagaagcaccgagcaaactgagcaccaccagttgtcataaaattgtgcgcgaagatctgcatgcaaatagcggtacagttgttgttcagagcgatctgagcgatccgaaactgaaagcaaccattacaggtcatcaggttaatggtacaccgctgacaccgagcagcctgtatgcagatcaggcaatgaccgttgccgattatctgtatcagcagctgcgtccgggtacagaaactccgggtctgaacgtttgtagcatggaagttaccaaaacactgattccgcagtatcctcctccggcaacaggtcagcatctgcaaattgaaggtaatgccgatctggaagcaagccaggtgaaaattacctttcgtaccgtgagcgcagatggtagcaaaattctggccgaacatgcagttggtatcgtgaaatatgaggatgttaatgcctggaaagaagagtggggtcgcattcagtatatggttcagagccagattgatatgttacagcagaaactggccaccggtgcggcacataaagttctgcgtggtatggcctataaactgtttaaagcactggttacctatgccgataactatcgcggtatggaagaggtgattctggatggtaaacagaccgaagcaaccgcaagcgttcagtttcagacgaccgaagccgatggtgaatttctgtgtagcccgtattggattgatagtctggcacatctgagcggttttattgttaatgcaagcgatcatctggatagcgaaaacagcgtgtatattagccatggttggggcagcattaaaatcgcaggtaaactgtcaccggaaaaaaagtatcgtagctatgtgcgtatgcaaccggcaccgggtaatattagcgtgggtgatgtttatatcatggatggcaccgaaattataggtatggttatgggtctgaaattccagaacattccgcgtcgtgcgctgaacattatgatgcctccggcaggtaaagcagcgagcagtcctgccgcaaaagccgttgcacgtccggctccgaaagcactgcctgccgcagctccggttaaaacccaggcaccagttccgaaagcggttaaagcaccgaaacctgttaaagccgttaaagttgcagccccagccggtgttaccagcaaagtgatgaatattgttgcccaagaaatcgatgtggatatgagtgaactggttgatgaagcagcctttgaaaatctgggtgttgattcactgctgagcctgaccatttcagcacgttttcgcgaagaactggatatggatattccgagcacactgtttaccgattgtagcaccgttggtgaactgaaaaaacactttagccagtttgatggtgccaccattgtggaagatgatagcagcgttacctcagatgaaccgagtggtgcaccgacaccgtttgaagcccctgcaggtcagagtgatagcacaccggcaagcagtgcaggtagtgatgatggtcatgatgatgttaaaccgtcagcaccgaccgaaggtggtgcatcactggcacgcaaactggttgcggaagaaatgggcgttgatgtgagtgaaattaccgatgatctggatctgaccgatattggtatggattcactgatgtctctgaccattctgggcagcatgcgtgaagccaccggtcgtgatctgcctgcggatttcctgaccgttaatgttaccattaaagacattgaaaccgcactgggtatgcgtccgcagcctaaagccgcaaaaccggcagcgaaagcaagcagcaaagcaccgcagctgagtgaagtgaacaaaaaactggcaagcctgccggatgttagcaatctgccaccggcatcaagcgttctgctgcagggcaatccgaaaacggcaaccaaaaaattctttctggttccggatggtagcggtagtgcaaccagctatattagcattccgaatattagcccgagcatggcagtttatggcctgaattgtccgtttatgaaatgtccggaaaaatggacctgtggtgttgaaggtgttagccgtctgtatctgaacgaaattaaacgtcgtcagccgagcggtccgtatctgattggcggttggagcgcaggcggtgttatggcgtatgaagttgcacagcagctggttaatgcgggtgaaaaagttgaaagcctggttctgattgatgcaccgtgtccggtggcgctggatccgctgccagcacgcctgcatatcttttttgatcagatcggcctgcttggtacaggtaaaccaggtggcaccccgagctggctgctgcctcattttgcaagcgcaattcagaacctgaaagattatgatccgacaccgatggatccgaaaattgcacctccggttctggcaatttggtgtaccgatggtgtttgtccgaatccagaagatcctcgtccgcctcctggtgaaggcgaagatccggcacctatgaaatggctgctgaataatcgtaccgattttagcgataatggttgggcacagctgctgccgaaagaaaactttcagtatgcagttatgggtggcaatcactttaccatgatgaaaggtgatcatggtgttaccctgggtaaactgattcagcagggcctgaaactgtaataa

SEQ ID NO: 10 - DNA, 807 bp, Artificial Sequence
  SEQ ID NO 10 (Brn1-EcoliOpt)
atggccaatattgaacagacctggtcactggcaggtaaagttgcagttgttaccggtagcggtcgtggtattggtaaagcaatggcaattgaactggcaaaacgtggtgcaaaagttgccgttaattatgcaaatgcagttgaaggtgcagaacaggtggtgaaagaaattaaagcattaggcaatggttcagatgcccatgcatttaaagcaaatgttggcaatgttgaagaaagcgagaaactgatggatgatgtggtgaaacattttggcaaactggatatttgttgcagcaatagcggtgttgttagctttggccattttaaagatgttacaccggaagaatttgaccgcgtgtttaacattaatacccgtggtcagttttttgttgccaaagcagcatataaacgcatggaaatgggtggtcgtattattctgatgggtagcattaccggtcaggcaaaaggtgttccgaaacatgcagtttatagcggtagcaaaggtgccattgaaacctttacacgttgtatggcagttgatgccggtgaaaaaaaagttaccgtgaattgtgttgcacccggtggtattaaaaccgatatgtatcatgccgtttgccgtgaatatatcccgaatggtgatcagctgagtgatgatcaggttgatgaatatgcatgcacatggtcaccgcataatcgtgttggtcagccgattgatattgcacgtgttgtttgttttctggcaagccaggatggtgattgggttaatggtaaagtgattggtattgatggtgcagcctgtatgtaataa

SEQ ID NO: 11 - DNA, 810 bp, Artificial Sequence
  SEQ ID NO 11 (Brn2-EcoliOpt)
atggttgttaccgttccgagcgcaagcctggaaggtcaggttgcactgattaccggtgcaggtcgtggtattggtcgtggttgtgcaattgaactgggtaaacgtggtgcaagcgttgttgttaattatgttagcagcgaaggtccggcaaatgaagtttgtgaaattattcgcggttttaacaatggtgcaaaagcagttgcaattcaggcagatgttagcaaagttagcgaaatccatcgtctgtttgaagaagcaaaacaggcctttggcaaaatcaatattgtgatgagcaatagcggcaccgaaagctgggataaaaccgaagaaattagcgaagagaaatacgaccatgtgtttaatctgaatacccgtgcgcagttttttgttggtcagaccgcctataaatacatcgaagataatggtcgcattatcctgatgagcagcattgcagcaggtctgctgggtgttaaagatcatgcactgtataatgcaagcaaaatggccgtgattggtttcattaaagcatttgcaaccgattttggtaaacgcggtgttaccgtgaatggtgttgcacctggtggtattaaaagcgatatgtttacacagaatgcctggcattatatcccaggtggtacaccggaatggcctgcagaaaaaattgaaagcctgatggcaagccattgtccgctgggtcgttgtgcagttccggaagatgttgcacgtgttgttgcatttctggcaagcgaagatggtggttgggttaatggtcaggttctgaccatttcaggtggtagcagccagtaataa

SEQ ID NO: 12 - DNA, 561 bp, Artificial Sequence
  SEQ ID NO 12 (Scd1-EcoliOpt)
atgttcgagaaaaaagaactgcagccgacctttgaagaagttatgggttgtcagagcgcatgttatgaatgggcagatagctatgatagcaaagattgggatcgtctgcgtaaatgtgttgcaccgacactgaaaattgattatcgtagcttcctggacaaaatgtgggaagcaatgcctgcagatgaatttgttgcaatggcaagcgatccggcagttctgggtaatccgctgctgaaaacccagcattttattggtggcacccgttgggaaaaaaccgcagaagatgaaattaccggttatcatcagctgcgtgttccgcatcagcgttataccgatgaaagccgtaccaccgttgcagttaaaggtcatgcacatagctttaacacccactggtacaaaaaaatcgatggcgaatggaaatttgcaggtctgaatccggatattcgctggtatgaatatgatttcgataaagtgtttgccgaaggtcgtgaacagctgggtgaagcaaaagcagcagcaggtattccggaaaccgcacctggtcaggcagtttaataa

SEQ ID NO: 13 - DNA, 1095 bp, Artificial Sequence
  SEQ ID NO 13 (CHS1-EcoliOpt)
atggcaaccctgtgtcgtccggaagttgcagttccggaatttatcattacccgtgaagataccctgaaactggcagaacgtctgcatgcacgtaatccgcagctgaaattagttctgcgtctgattaatggcaccggtgttcagaaacgtcatattgttcagccgctggaacaggcactggcacatccgggttttgaaattcgtaatcgtatttatgaacgcgaagccaaacgtcgtgttccgcctgttattcgtcaggccctggaaaatgcagaactgaccgcacgtgatattgatgccattatctttgttagctgcaccggttttatgatgccgagcatgaccgcatatctgattaacgcaatgggttttcgtcatgatacccgtcagattccgatggcacagctgggttgtgcagcgggtgcagcagcaattagccgtgcacatgatttttgtgcagcacatgccgaagcaaatgttctgattgttgcatgtgaattttgcagcctgtgttatcagccgaccgatgatgatgttggtagcctgctgagtgatggtctgtttggtgatgcagttggtgcagttgttgttcgtggtagcggtggtacaggtattcgtctggaacgtaatgaagcctatctgattccggataccgaagaatggattagctatgcagttaaagataccggctttcattttcgcctggataaacgtgttccgggtacaatgaaaccgctggctccggttatgcgtcgtctggttgcaagccatggttgggatgtgggtaatctggatttttacattgttcatgccggtggtccgcgtattctgaatgatctgtgtaaatttctggaagttccgcaagaagcatttcgttatagccgtgcgaccctgaccgaatatggtaatattgcaagcgcagttgttctggatagcctgggtcgtctgtttgccgatcgtccgatgccggatggtgcacgtggtgttattgcaggttttggtccgggtattaccgcagaaatggcagttggtagctgggttaatgatcatggtcagagccgtggtggtgcccgtagcagcagcgcagcataataa

SEQ ID NO: 14 - DNA, 966 bp, Artificial Sequence
  SEQ ID NO 14 (BdsA-EcoliOpt)
atgaccaatgcaagcctgaaaaccccgagcctgcgttttaccttaggtggtgatcgtccggtttgtcgtatgggttatggtgcaatgtatctgaccggtcctggttattggggtccgcctgcagatccggatgcagcagttcgtctgctgcgtcgtgcagttgatctgggtgttaactttattgataccgcagatgcatatggtccggataccaatgagcagattattcgtcgtgcactgcatccgtatccggcaggtctggttattaccaccaaaggtggcctgctgcgtagcggtccggaagattggaccgttcagggtcgtccgtatattgttccgtgtggtcgtccggcatatctgcgtcagcaggttgaaatgagtctgcgtaatctgggtttagaacgcattgaactgtatcagctgcatcgtattgatccggcagttccgctggcagatcagctgggtgaactggcacgtctgaaagaacagggtaaaattcatcatattggtctgagcggtcagccggaagttaccgttgaacagctggcaaaagcacgtgaaattaccgaaattgcagccgtggaaaatctgtataatgttgcagatcgtagcggtgaaaaagcactgcgttatgcagaacagcatggtatggcatttattccgtggtttccgatgggtcatggtgatctggcaaaaccgggtagcgttctgagtggtctggccaaagaacgtggtgcaacaccggcacagctggccctggcatggctgctgcaccgtagtccgagcattctggcaattccgggtacaagcagcattaaacatctggaagaaaattgtcgtgcagccgatatgaaactggatgaaagcgaactggcagcaattgaagatgcagccgcagcagcaaaaacctggcgtccgagcgcaagcgaaagccgtgcagcacataccaaagaaaccccgtaataa

SEQ ID NO: 15 - DNA, 336 bp, Artificial Sequence
  SEQ ID NO 15 (BdsB-EcoliOpt)
atggttctgcataccgcacatcgtgaactgtgtcgtgatcgtgcagcaggtcgtgttgttggtaaagcagccgcagcaacccatgaacgtgaactgattcgtatgaccggtggcaccctgcgtatggatgttcagcatatttttgccaacgatcattttggtgcagcatttggtgttctgcgtgcacatttaggtggtgcaagcctggcaatgccgttttgtggtgtttggcgttttcgtggtggtctgattaccgaacattgggaaaatgcatatgacgttgcaagcgttatggcatttctgggtgatgaaagcggtgcacctccggcataataa

SEQ ID NO: 16 - DNA, 1122 bp, Artificial Sequence
  SEQ ID NO 16 (RppA-EcoliOpt)
atggcaaccctgtgtcgtccggcaattgcagtgccggaacatgttattaccatgcagcagaccctggatctggcacgtgaaacccatgcaggtcatccgcagcgtgatctggttctgcgtctgattcagaataccggtgttcagacccgtcatctggttcagccgattgaaaaaaccctggcacatccgggttttgaagttcgtaatcaggtttatgaagccgaagcaaaaacccgtgttccggaagttgttcgtcgtgcactggcaaatgcagaaaccgaaccgagcgaaattgatctgattgtttatgttagctgcaccggttttatgatgccgagcctgaccgcatggattattaacagtatgggttttcgtccggaaacacgtcagctgccgattgcacagctgggttgtgcagccggtggtgcagcaattaatcgtgcacatgatttttgtgttgcctatccggattcaaatgttctgattgtgagctgtgaattttgcagcctgtgttatcagccgaccgatattggtgttggtagcctgctgagcaatggtctgtttggtgatgcactgagcgcagccgttgttcgtggtcaaggtggcaccggtatgcgtctggaacgtaatggtagccatctggtgccggataccgaagattggattagctatgcagttcgtgataccggttttcattttcagctggataaacgcgttccgggtacaatggaaatgctggctccggttctgctggacctggttgatctgcatggttggagcgttccgaatatggattttttcattgttcatgccggtggtccgcgtattctggatgatctgtgtcattttctggatctgcctccggaaatgtttcgttatagccgtgcaaccctgaccgaacgtggtaatattgcaagcagcgttgtttttgatgccctggcacgcctgtttgatgatggtggtgccgcagaaagcgcacagggtctgattgcaggttttggtccgggtattaccgcagaagttgcagttggtagctgggcaaaagaaggtctgggagcagacgttggtcgcgatctggatgaactggaactgaccgcaggcgttgccctgagcggttaataa

SEQ ID NO: 17 - DNA, 2010 bp, Artificial Sequence
  SEQIDNO17(copA-II,Pseudomonasputida)
tcattcgtccacgcggacttctcggaacatgcccatttccatatggaacagtaggtggcagtggtaggcccaacggcccaaagcatccgcggttactcggtagcttcgttttgagcctggaggcatgtcgatggtgtgttttcgaaccatgaaattaccgtcctcatcctccagatcgctccacattccgtggaggtggatggggtgagtcatcatggtgtcgttgaccaaagtgattcgcagacgctcgccatatttgaggcgtaggggttcagcatcggaaaatttgacgccgtcaaatgaccaggcgaacttctccatatggcctgtgagatgcagctcgatggtgcgccctggttcacgtccatctggatcaatgaacgtgcttcgcagatccgcataggtcaggacacgacgtccattgttgcgcaagccgattcccggatcgtccaacttaggcgttggactcatagtctgcatgtcgaccaaggggttgttagtctctgaggctgggtgagactgcatggcgccacccataccttccatgctgctgtggtccatatcggccatgccgccttggtccatcccagccatcttgctatggtccatatcggccatgccgccttggtccatcccagccatcttgctgtggtccatgccggccatgccgccgtggtccatctcagccattttgctgtgatccatgccggccatgctgccatggtccatcccagccattttgctgtggtccatgccggtcatgttgccgtggtccatcccagccattttgctgtggtccatgccggccatgttgccgtggtccatcccagccattttgctgtggtccataccggccatgttgccgtggtccatgccggccatgctgccgtgatccatacccatatcactcattgagatgagagggcgcggatcaactgccggaactgcagcttgcataccttcgcgaacggctaaagtgcctctggagtagcctgtgcgatccatggattgagcaaaaatggtgtaagcctgctcgtcttccggctcaacaatgacgtcgtaggtttcggcaacggcgatgcggaactcgtcgaccgcaacgcgtttgacatgctgcccatccgccgcgacaaccgtcatcttcaggccgggaattcgcacatcgaaatacgtcatggccgagccattgatgaagcgcaggcggattttctcgcccggcttgaacacgcccgtccagttaccgtccggggcctggccgttcatcaggtaggtgtaggtgtagccgctcacatcagcgagatcggtggggctcattttcatctcggcccacatcttgcggtcggctaccgcggcggaccagcccatttcgctcacatcgttgacgaaatcgcccaccgtgcgcttgtggtagttgtagtagtcggactgcttcttgagcttggacagcacccgtgcagggtcttcatccgtccagtcgctgagcatgaccacgtagtcccggtcataggtgaacggctcaggttctttcgcatcgatgaccagggcaccatacaccccgacctgctcctggaaaccggagtggctgtggtaccagtaggtgccgttctgctggaccttgaacttgtactcatacatgccgtctggggcgatgccatggaagctcaggcccggcacgccgtccatgttcgccggcaggatgatgccgtgccagtggatggaggtgtcctgctgcaagcggttgcgcacacgcagcgtgacggtatcgccctcgcgccagcgcaggatgggcccgggaatggagccgttgattgccatcgctgtacgtacagtgccggtaatgttgaccggcaactcaccgatgtagagatcaaaatcggtgcctgtcagaacattcggttggccagggctggtcacagcccagaccggcgcgcgccacatgcccagtccacccagtaatccagtagcggccaggcctttgacgaaagatcgtctcgtggttttgctttgcat

SEQ ID NO: 18 - DNA, 1551 bp, Artificial Sequence
  SEQIDNO18(cueO,EscherichiacoliBL21(DE3))
atgcaacgtcgtgatttcttaaaatattccgtcgcgctgggtgtggcttcggctttgccgctgtggaaccgcgcagtatttgcggcagaacgcccaacgttaccgatccctgatttgctcacgaccgatgcccgtaatcgcattcagttaactattggcgcaggccagtccacctttggcgggaaaactgcaactacctggggctataacggcaatctgctggggccggcggtgaaattacagcgcggcaaagcggtaacggttgatatctacaaccaactgacggaagagacaacgttgcactggcacgggctggaagtaccgggtgaagtcgacggcggcccgcagggaattattccgccaggtggcaagcgctcggtgacgttgaacgttgatcaacctgccgctacctgctggttccatccgcatcagcacggcaaaaccgggcgacaggtggcgatggggctggctgggctggtggtgattgaagatgacgagatcctgaaattaatgctgccaaaacagtggggtatcgatgatgttccggtgatcgttcaggataagaaatttagcgccgacgggcagattgattatcaactggatgtgatgaccgccgccgtgggctggtttggcgatacgttgctgaccaacggtgcaatctacccgcaacacgctgccccgcgtggttggctgcgcctgcgtttgctcaatggctgtaatgcccgttcgctcaatttcgccaccagcgacaatcgcccgctgtatgtgattgccagcgacggtggtctgctacctgaaccagtgaaggtgagcgaactgccggtgctgatgggcgagcgttttgaagtgctggtggaggttaacgataacaaaccctttgacctggtgacgctgccggtcagccagatggggatggcgattgcgccgtttgataagcctcatccggtaatgcggattcagccgattgctattagtgcctccggtgctttgccagacacattaagtagcctgcctgcgttaccttcgctggaagggctgacggtacgcaagctgcaactctctatggacccgatgctcgatatgatggggatgcagatgctaatggagaaatatggcgatcaggcgatggccgggatggatcacagccagatgatgggccatatggggcacggcaatatgaatcatatgaaccacggcgggaagttcgatttccaccatgccaacaaaatcaacggtcaggcgtttgatatgaacaagccgatgtttgcggcggcgaaagggcaatacgaacgttgggttatctctggcgtgggcgacatgatgctgcatccgttccatatccacggcacgcagttccgtatcttgtcagaaaatggcaaaccgccagcggctcatcgcgcgggctggaaagataccgttaaggtagaaggtaatgtcagcgaagtgctggtgaagtttaatcacgatgcaccgaaagaacatgcttatatggcgcactgccatctgctggagcatgaagatacggggatgatgttagggtttacggtataa

(19) United States
(12) Patent Application Publication
(10) Pub. No.: US 2024/0060096 A1
(43) Pub. Date: Feb. 22, 2024

(54) COMPOSITIONS AND METHODS FOR PRODUCING 1,8-DIHYDROXYNAPHTHALENE AND CYCLIC HYDROCARBONS FROM 1,8-DIHYDROXYNAPHTHALENE

(71) Applicant: Technology Holding, LLC, Salt Lake City, UT (US)

(72) Inventors: Mukund Karanjikar, Suwanee, GA (US); Robert Price, Cottonwood Heights, UT (US); Younghwan Kim, Salt Lake City, UT (US); Oyvind Hatlevik, Holladay, UT (US)

(21) Appl. No.: 17/556,966

(22) Filed: Dec. 20, 2021

Related U.S. Application Data
(60) Provisional application No. 63/127,990, filed on Dec. 18, 2020.

(51) Int. Cl. C12P 7/22; C12N 9/04; C12N 9/88; C12N 9/10; C12N 1/20

(52) U.S. Cl. CPC C12P 7/22; C12N 9/0006; C12N 9/88; C12N 9/1029; C12N 1/205; C12R 2001/40

(57) ABSTRACT

A method of producing 1,8-dihydroxy naphthalene (DHN) is provided. The method includes culturing cells under suitable culture conditions for the production of DHN. The cells include a nucleic acid encoding a polyketide synthase polypeptide and one or more nucleic acids encoding one or more protein enzymes used in the DHN melanin pathway. DHN melanin pathway genes may be expressed in the cells, which may recombinant E. coli cells. The cells may include inhibitors to stop the conversion of DHN into melanin within the cells. The cells may include glucose as a cell nutrient. The glucose may be derived from biomass. DHN may be produced, harvested and the harvested DHN may be catalyzed into a cyclic hydrocarbon.

### CROSS-REFERENCE TO RELATED APPLICATION

This application claims the benefit of U.S. Provisional Application No. 63/127,990 filed on Dec. 18, 2020.

### TECHNICAL FIELD

This disclosure pertains to compositions and methods for producing 1,8-dihydroxynaphthalene (DHN) and cyclic hydrocarbons based on DHN. More specifically, the disclosure pertains to production of DHN through the expression of proteins in a host cell using genes encoding proteins related to the production of DHN and carbon neutral starting materials. The disclosure also pertains to making cyclic hydrocarbons using DHN.

### BACKGROUND

Cyclic hydrocarbons are a critical starting material for a variety of products that benefit from high energy density materials such as jet fuels. They are also used to make lubricants and other commercially important products. One issue with the production of hydrocarbons is that they are typically made from expensive starting materials. Additionally, the starting materials for making cyclic hydrocarbons are not carbon-neutral.

A less expensive starting material that may be used to make cyclic hyrdocarbons is 1,8-dihydroxynaphthalene (DHN). However, although DHN is produced in nature, the harvesting of natural sources of DHN is inefficient and not environmentally friendly. Even when overcoming the inefficiencies of harvesting natural DHN through biosynthesizing DHN, the starting materials used in the process may not be carbon-neutral. Further, DHN naturally tends to become Melanin.

Thus, more economical, carbon-neutral methods for producing cyclic hydrocarbons and DHN are needed. In particular, methods that produce DHN at rates, titers, and purity that are sufficient to meet the demands of a robust commercial process are desirable. Also desired are systems for producing DHN and cyclic hydrocarbons from inexpensive carbon-neutral starting materials.

### SUMMARY

Embodiments of the present invention include cell compositions and methods for producing 1,8-dihydroxy naphthalene (DHN). A method includes culturing cells under suitable culture conditions for the production of DHN and producing DHN. In one embodiment, the cells may include a nucleic acid encoding a polyketide synthase polypeptide. The cells may also include one or more nucleic acids encoding one or more proteins used in the production of DHN. In another embodiment, the cells may include a duplicate copy of an endogenous nucleic acid encoding a polyketide synthase polypeptide and a duplicate copy of one or more endogenous nucleic acids encoding one or more proteins used in the production of DHN

The nucleic acids encoding a polyketide synthase may include nucleic acids encoding a type I polyketide synthase. In another embodiment, the nucleic acids encoding a polyketide synthase may include nucleic acids encoding a type III polyketide synthase. Thus, in some embodiments, the cells may include nucleic acids encoding a fungal polyketide synthase polypeptide. In other embodiments, the cells may include nucleic acids encoding a bacterial polyketide synthase polypeptide.

The cells may include nucleic acids encoding proteins may be found in a DHN melanin pathway occurring in nature. In one embodiment, the nucleic acids may encode one or more proteins found in the DHN melanin pathway found in Cochliobolus heterostrophus. In other embodiments, the nucleic acids may encode one or more proteins found in the DHN melanin pathway found in Sorangium cellulosum. In other embodiments, the nucleic acids may encode one or more proteins found in the DHN melanin pathway found in Streptomyces griseus. The cells may include one or more nucleic acids encoding the proteins of an entire DHN melanin pathway.

The nucleic acids may be in the form of genes that express the proteins of various DHN melanin pathways in a recombinant host cell. Accordingly, in some embodiments the cells include recombinant host cells, nucleic acids for encoding proteins found in a DHN melanin pathway, and the expressed proteins themselves.

In one embodiment, the nucleic acids encoding a polyketide synthase polypeptide may include one or more of PKS18, SoceCHS1, RppA and PKSwd. These polyketide synthase genes may express the polyketide synthase that may convert Acetyl CoA into 1,3,6,8-tetrahydroxynapthalene (T4HN). The nucleic acids that encode one or more proteins used in the production of DHN may include one or more of BRN1, BRN2, SCD1, BdsA, and BdsB. These genes may express proteins in the form of enzymes to facilitate the conversion of T4HN into DHN. By way of nonlimiting example, the gene BRN2 may express the enzyme T4HN reductase that helps convert T4HN into Scytalone along the Cochliobolus heterostrophus DHN melanin pathway. The gene SCD1 may express the enzyme scytalone dehydratase that helps convert Scytalone into 1,3,8-trihydroxynapthalene (T3HN). The gene BRN1 may express the enzyme T3HN reductase that helps convert T3HN into Vermelone. The scytalone dehydratase expressed by SCD1 may also help convert Vermelone into DHN along the Cochliobolus heterostrophus DHN melanin pathway. Similarly, the nucleic acids BdsA and BdsB are genes that express bacterial DHN (dihydroxynaphthalene) synthase A and bacterial DHN (dihydroxynaphthalene) synthase B that facilitate the conversion of T4HN into DHN along the Sorangium cellulosum and Streptomyces griseus DHN melanin pathways.

The cells of the embodiments of the present invention include host cells in which the genes and/or nucleic acids described herein throughout may be expressed to form proteins along the various DHN melanin pathways. In certain embodiments, the host cells may include at least one cell chose from gram-positive bacterial cells, gram-negative bacterial cells, fungal cells, filamentous fungal cells, algal cells and yeast cells.

The cells of embodiments of the present invention may include nucleotide sequences of the genes described herein that are codon-optimized for expression in the host cell.

Culturing the cells may include providing a feedstock for the host cells. In one embodiment, the feedstock may include glucose. The glucose may be derived from biomass.

Producing the DHN may include limiting or inhibiting melanogenesis, or the conversion of DHN to melanin, which it naturally wants to do. In one embodiment, the inhibition of DHN conversion to melanin includes inhibiting expression of p-diphenol oxidase enzymes in the cells. In another embodiment, the inhibition of DHN conversion to melanin includes removing a gene that can express p-diphenol oxidase in the cells. Accordingly, cells of embodiments of the present invention may also include one or more melanogenesis inhibition compounds.

Embodiments of the present invention also include methods for producing cyclic hydrocarbons from DHN or from biomass which can be converted into sugar for feeding cells that contain nucleic acids encoding proteins used in the production of DHN. These methods may include the steps in embodiments of methods for producing DHN described herein throughout, along with cell compositions described herein throughout. The methods may also include converting DHN into cyclic hydrocarbons through one or more of catalysis, dehydration, and hydrolyzation.

Accordingly, embodiments described herein describe compositions and methods to produce 1,8-dihydroxynaphthalene (DHN) in a more cost-efficient, carbon-neutral manner, with the produced DHN being used to make cyclic hydrocarbons. Additionally, embodiments described here describe ways to make cyclic hydrocarbons from biomass.

### BRIEF DESCRIPTION OF THE DRAWINGS

FIG. 1 is a flow chart showing an overall scheme for producing of 1,8-dihydroxynaphthalene (DHN);

FIG. 2 is a schematic diagram depicting certain DHN-Melanin pathways found in nature;

FIGS. 3A-3E is the nucleotide sequence of PKS18, a polyketide synthase gene codon-optimized for expression in Pseudomonas putida (P. putida) (SEQ ID NO:1);

FIG. 4 is the nucleotide sequence of Brn1, a 1,3,8-trihydroxynapthalene (T3HN) reductase gene codon-optimized for expression in P. putida (SEQ ID NO: 2);

FIG. 5 is the nucleotide sequence of a Brn2, 1,3,6,8-tetrahydroxynapthalene (T4HN) reductase gene codon-optimized for expression in P. putida (SEQ ID NO: 3);

FIG. 6 is the nucleotide sequence of Scd1, a scytalone dehydratase gene codon-optimized for expression in P. putida (SEQ ID NO: 4);

FIG. 7 is the nucleotide sequence of CHS1, a polyketide synthase gene codon-optimized for expression in P. putida (SEQ ID NO: 5);

FIG. 8 is the nucleotide sequence of BdsA, a bacterial DHN synthase A gene codon-optimized for expression in P. putida (SEQ ID NO: 6);

FIG. 9 is the nucleotide sequence of BdsB, a bacterial DHN synthase B gene codon-optimized for expression in P. putida (SEQ ID NO: 7);

FIG. 10 is the nucleotide sequence of RppA, a polyketide synthase gene codon-optimized for expression in P. putida (SEQ ID NO: 8);

FIGS. 11A-11E is the nucleotide sequence of PKS18, a polyketide synthase gene codon-optimized for expression in Escherichia coli (E. coli) (SEQ ID NO:9);

FIG. 12 is the nucleotide sequence of Brn1, a 1,3,8-trihydroxynapthalene (T3HN) reductase gene codon-optimized for expression in E. coli (SEQ ID NO: 10);

FIG. 13 is the nucleotide sequence of a Brn2, 1,3,6,8-tetrahydroxynapthalene (T4HN) reductase gene codon-optimized for expression in E. coli (SEQ ID NO: 11);

FIG. 14 is the nucleotide sequence of Scd1, a scytalone dehydratase gene codon-optimized for expression in E. coli (SEQ ID NO: 12);

FIG. 15 is the nucleotide sequence of CHS1, a polyketide synthase gene codon-optimized for expression in E. coli (SEQ ID NO: 13);

FIG. 16 is the nucleotide sequence of BdsA, a bacterial DHN synthase A gene codon-optimized for expression in E. coli (SEQ ID NO: 14);

FIG. 17 is the nucleotide sequence of BdsB, a bacterial DHN synthase B gene codon-optimized for expression in E. coli (SEQ ID NO: 15);

FIG. 18 is the nucleotide sequence of RppA, a polyketide synthase gene codon-optimized for expression in E. coli (SEQ ID NO: 16);

FIG. 19 is the nucleotide sequence of copA-11, a p-diphenol oxidase gene codon-optimized for expression in P. putida (SEQ ID NO: 17) shown in reverse form;

FIG. 20 is the nucleotide sequence of cueO, a p-diphenol oxidase gene codon-optimized for expression in E. coli (SEQ ID NO: 18);

FIGS. 21A and 21B are respective representations of single and dual vector systems for certain gene combinations for a DHN pathway;

FIG. 22 is a chart showing culture test results of single- and dual-vector system transformants in Luria-Bertani (LB) liquid media at 37° C. for 24 hours without Isopropyl p-D-1-thiogalactopyranoside (IPTG) induction;

FIG. 23 is a schematic diagram depicting another DHN-Melanin pathway utilizing a polyketide synthase gene found in Aspergillus nidulans;

FIGS. 24A and B are subcloning gel images and sequencing analysis for the polyketide synthase gene of FIG. 23;

FIGS. 25A and B are charts showing culture test results for single vector system transformants with IPTG induced samples;

FIG. 26 is a chart showing IPTG concentrations for various DHN pathway genes with a chart showing gene pathway nomenclature;

FIG. 27 is a schematic diagram depicting the function of Laccase as part of the DHN melanin pathway;

FIGS. 28A and B are flow diagrams of laccase knockout strategies using the laccase coding genes multicopper oxidase (CueO) and polyphenol oxidase (yfiH) respectively;

FIG. 29 is a schematic diagram showing the ΔyfiH E. coli strain removal;

FIG. 30 is a schematic diagram showing the ΔcueO E. coli strain removal;

FIG. 31 shows the PCR amplification results confirming melanin inhibition through laccase enzyme gene removal;

FIG. 32 shows the results of antibiotics resistance tests for ΔcueO and ΔyfiH;

FIG. 33 shows the high performance liquid chromatography (HPLC) test results of four E. coli colony samples where melanin inhibition by gene removal was utilized;

FIG. 34 shows a schematic flow diagram for a method of producing DHN;

FIG. 35 shows a schematic flow diagram for a method of producing cyclic hydrocarbons from sugars using a DHN intermediary;

FIG. 36 shows a schematic flow diagram for a method of producing jet fuel from biomass;

FIG. 37 is a map of part 1 of pks18 optimized for E. coli;

FIG. 38 is a map of part 2 of pks18 optimized for E. coli;

FIG. 39 is a map of rppA optimized for E. coli;

FIG. 40 is a map of bdsA and bdsB optimized for E. coli;

FIG. 41 is a map of Chs1 optimized for E. coli;

FIG. 42A is a map of Scd1 and brn1 optimized for E. coli;

FIG. 42B is a map of brn2 optimized for E. coli;

FIG. 43 is a map of part 1 of pks18 optimized for P. putida;

FIG. 44 is a map of part 2 of pks18 optimized for P. putida;

FIG. 45 is a map of rppA optimized for P. putida;

FIG. 46 is a map of bdsA and bdsB optimized for P. putida;

FIG. 47 is a map of Chs1 optimized for P. putida; and,

FIG. 48 is a map of Scd1, brn1, and brn2 optimized for P. putida.

### DETAILED DESCRIPTION

### Definitions

The term “about” is used herein to mean approximately, roughly, around, or in the region of. When the term “about” is used in conjunction with a numerical range, it modifies that range by extending the boundaries above and below the numerical values-set forth. In general, the term “about” is used herein to modify a numerical value above and below the stated value by a variance of 20 percent up or down (higher or lower), preferably 15 percent, more preferably 10 percent and most preferably 5 percent.

When introducing elements of aspects of the invention or aspects thereof, the articles “a,” “an,” “the,” and “said” are intended to mean that there are one or more of the elements. The terms “comprising,” “including,” and “having” are inclusive and mean that there may be additional elements other than the listed elements.

The word “exemplary” is used herein to mean “serving as an example or illustration.” Any aspect or design described herein as “exemplary” is not necessarily to be construed as preferred or advantageous over other aspects or designs.

The terms “A or B,” “at least one of A and B,” “one or more of A and B”, or “A and/or B” as used herein include all possible combinations of items enumerated with them. For example, use of these terms, with A and B representing different items, means: (1) including at least one A; (2) including at least one B; or (3) including both at least one A and at least one B. In addition, the articles “a” and “an” as used herein should generally be construed to mean “one or more” unless specified otherwise or clear from the context to be directed to a singular form.

The term “genome” or “genomic DNA” is referring to the heritable genetic information of a host organism. Said genomic DNA comprises the entire genetic material of a cell or an organism, extrachromosomal DNA, and organellar DNA.

The term “promoter” refers to a polynucleotide which directs the transcription of a structural gene to produce mRNA. Typically, a promoter is located in the 5′ region of a gene, proximal to the start codon of a structural gene. If a promoter is an inducible promoter, then the rate of transcription increases in response to an inducing agent. In contrast, the rate of transcription is not regulated by an inducing agent, if the promoter is a constitutive promoter.

The term “enhancer” refers to a polynucleotide. An enhancer can increase the efficiency with which a particular gene is transcribed into mRNA irrespective of the distance or orientation of the enhancer relative to the start site of transcription. Usually, an enhancer is located close to a promoter, a 5′-untranslated sequence or in an intron.

A nucleotide is “heterologous to” an organism or a second nucleotide if it originates from a foreign species, or, if from the same species, is modified from its original form. For example, a promoter operably linked to a heterologous coding sequence refers to a coding sequence from a species different from that from which the promoter was derived, or, if from the same species, a coding sequence which is not naturally associated with the promoter (e. g. a genetically engineered coding sequence or an allele from a different ecotype or variety).

“Transgene”, “transgenic” or “recombinant” refers to a nucleotide manipulated by man or a copy or complement of a nucleotide manipulated by man. For instance, a transgenic expression cassette comprising a promoter operably linked to a second nucleotide may include a promoter that is heterologous to the second nucleotide as the result of manipulation by man (e.g., by methods described in Sambrook et al., Molecular Cloning—A Laboratory Manual, Cold Spring Harbor Laboratory, Cold Spring Harbor, N.Y., (1989) or Current Protocols in Molecular Biology, Volumes 1-3, John Wiley & Sons, Inc. (1994-1998)) of an isolated nucleic acid comprising the expression cassette. In another example, a recombinant expression cassette may comprise polynucleotides combined in such a way that the polynucleotides are extremely unlikely to be found in nature. For instance, restriction sites or plasmid vector sequences manipulated by man may flank or separate the promoter from a second nucleotide. Plasmids, bacteriophages (such as phage λ), cosmids, and bacterial artificial chromosomes (BACs) may be used as cloning vectors in bacterial hosts such as E. coli. If using larger organisms such as yeast, suitable artificial chromosomes such as yeast artificial chromosomes (YACs) may be used. One of skill will recognize that nucleotides can be manipulated in many ways and are not limited to the examples above.

In case the term “recombinant” is used to specify an organism or cell, e.g., a microorganism, it is used to express that the organism or cell comprises at least one “transgene”, “transgenic” or “recombinant” nucleotide, which is usually specified later on.

A nucleotide “exogenous to” an individual organism is a nucleotide which is introduced into the organism by any means other than by a sexual cross.

The terms “operable linkage” or “operably linked” are generally understood as meaning an arrangement in which a genetic control sequence, e.g., a promoter, enhancer or terminator, is capable of exerting its function with regard to a polynucleotide being operably linked to it, for example a polynucleotide encoding a polypeptide. Function, in this context, may mean for example control of the expression, i.e., transcription and/or translation, of the nucleic acid sequence. Control, in this context, encompasses for example initiating, increasing, governing or suppressing the expression, i.e., transcription and, if appropriate, translation. Controlling, in turn, may be, for example, tissue- and/or time-specific. It may also be inducible, for example by certain chemicals, stress, pathogens and the like. Preferably, operable linkage is understood as meaning for example the sequential arrangement of a promoter, of the nucleic acid sequence to be expressed and, if appropriate, further regulatory elements such as, for example, a terminator, in such a way that each of the regulatory elements can fulfill its function when the nucleic acid sequence is expressed. An operably linkage does not necessarily require a direct linkage in the chemical sense. Genetic control sequences such as, for example, enhancer sequences are also capable of exerting their function on the target sequence from positions located at a distance to the polynucleotide, which is operably linked. Preferred arrangements are those in which the nucleic acid sequence to be expressed is positioned after a sequence acting as promoter so that the two sequences are linked covalently to one another. The distance between the promoter and the amino acid sequence encoding polynucleotide in an expression cassette, is preferably less than 200 base pairs, especially preferably less than 100 base pairs, very especially preferably less than 50 base pairs. The skilled worker is familiar with a variety of ways in order to obtain such an expression cassette. However, an expression cassette may also be constructed in such a way that the nucleic acid sequence to be expressed is brought under the control of an endogenous genetic control element, for example an endogenous promoter, for example by means of homologous recombination or else by random insertion. Such constructs are likewise understood as being expression cassettes for the purposes of the invention.

The term “expression cassette” means those constructs in which the nucleic acid sequence encoding an amino acid sequence to be expressed is operably linked to at least one genetic control element which enables or regulates its expression (i.e., transcription and/or translation). The expression may be, for example, stable or transient, constitutive or inducible.

The terms “express,” “expressing,” “expressed” and “expression” refer to expression of a gene product (e.g., a biosynthetic enzyme of a gene of a pathway or reaction defined and described in this application) at a level that the resulting enzyme activity of this protein encoded for, or the pathway or reaction that it refers to allows metabolic flux through this pathway or reaction in the organism in which this gene/pathway is expressed in. The expression can be done by genetic alteration of the microorganism that is used as a starting organism. In some embodiments, a microorganism can be genetically altered (e.g., genetically engineered) to express a gene product at an increased level relative to that produced by the starting microorganism or in a comparable microorganism which has not been altered. Genetic alteration includes, but is not limited to, altering or modifying regulatory sequences or sites associated with expression of a particular gene (e.g. by adding strong promoters, inducible promoters or multiple promoters or by removing regulatory sequences such that expression is constitutive), modifying the chromosomal location of a particular gene, altering nucleic acid sequences adjacent to a particular gene such as a ribosome binding site or transcription terminator, increasing the copy number of a particular gene, modifying proteins (e.g., regulatory proteins, suppressors, enhancers, transcriptional activators and the like) involved in transcription of a particular gene and/or translation of a particular gene product, or any other conventional means of deregulating expression of a particular gene using routine in the art (including but not limited to use of antisense nucleic acid molecules, for example, to block expression of repressor proteins).

In some embodiments, a microorganism can be physically or environmentally altered to express a gene product at an increased or lower level relative to level of expression of the gene product unaltered microorganism. For example, a microorganism can be treated with, or cultured in the presence of an agent known, or suspected to increase transcription of a particular gene and/or translation of a particular gene product such that transcription and/or translation are enhanced or increased. Alternatively, a microorganism can be cultured at a temperature selected to increase transcription of a particular gene and/or translation of a particular gene product such that transcription and/or translation are enhanced or increased.

The terms “deregulate,” “deregulated” and “deregulation” refer to alteration or modification of at least one gene in a microorganism, wherein the alteration or modification results in increasing efficiency of production of a given compound in the microorganism relative to production in absence of the alteration or modification. In some embodiments, a gene that is altered or modified encodes an enzyme in a biosynthetic pathway, or a transport protein, such that the level or activity of the biosynthetic enzyme in the microorganism is altered or modified, or that the transport specificity or efficiency is altered or modified. In some embodiments, at least one gene that encodes an enzyme in a biosynthetic pathway, i.e., a polypeptide bringing about a specific activity in the biosynthetic pathway, is altered or modified such that the level or activity of the enzyme is enhanced or increased relative to the level in presence of the unaltered or wild type gene.

Deregulation also includes altering the coding region of one or more genes to yield, for example, an enzyme that is feedback resistant or has a higher or lower specific activity. Also, deregulation further encompasses genetic alteration of genes encoding transcriptional factors (e.g., activators, repressors) which regulate expression of genes coding for enzymes or transport proteins. The terms “deregulate,” “deregulated” and “deregulation” can further be specified in regard to the kind of deregulation present.

In case the particular activity, is altered or modified such that the level or activity of the enzyme is enhanced or increased relative to the level in presence of the unaltered or wild type gene, the term “up-regulated” is used. In case particular activity, is altered or modified such that the level or activity of the enzyme is lowered or decreased relative to the level in presence of the unaltered or wild type gene, the term “down-regulated” is used.

The term “deregulated” includes expression of a gene product at a level lower or higher than that expressed prior to manipulation of the microorganism or in a comparable microorganism which has not been manipulated. In one embodiment, the microorganism can be genetically manipulated (e.g., genetically engineered) to express a level of gene product at a lesser or higher level than that expressed prior to manipulation of the microorganism or in a comparable microorganism which has not been manipulated. Genetic manipulation can include, but is not limited to, altering or modifying regulatory sequences or sites associated with expression of a particular gene (e.g., by removing strong promoters, inducible promoters or multiple promoters), modifying the chromosomal location of a particular gene, altering nucleic acid sequences adjacent to a particular gene such as a ribosome binding site or transcription terminator, decreasing the copy number of a particular gene, modifying proteins (e.g., regulatory proteins, suppressors, enhancers, transcriptional activators and the like) involved in transcription of a particular gene and/or translation of a particular gene product, or any other conventional means of deregulating expression of a particular gene routine in the art (including but not limited to use of antisense nucleic acid molecules, or other methods to knock-out or block expression of the target protein).

The term “deregulated gene activity” also means that a gene activity is introduced into a microorganism where the respective gene activity, has not been observed before, e.g., by introducing a recombinant gene, e.g., a heterologous gene, in one or more copies into the microorganism preferably by means of genetic engineering.

The phrase “deregulated pathway or reaction” refers to a biosynthetic pathway or reaction in which at least one gene that encodes an enzyme in a biosynthetic pathway or reaction is altered or modified such that the level or activity of at least one biosynthetic enzyme is altered or modified. The phrase “deregulated pathway” includes a biosynthetic pathway in which more than one gene has been altered or modified, thereby altering level and/or activity of the corresponding gene products/enzymes. In some cases the ability to “deregulate” a pathway (e.g., to simultaneously deregulate more than one gene in a given biosynthetic pathway) in a microorganism arises from the particular phenomenon of microorganisms in which more than one enzyme (e.g., two or three biosynthetic enzymes) are encoded by genes occurring adjacent to one another on a contiguous piece of genetic material termed a “cluster” or “gene cluster” In other cases, in order to deregulate a pathway, a number of genes must be deregulated in a series of sequential engineering steps.

To express the deregulated genes according to the invention, the DNA sequence encoding the polypeptide must be operably linked to regulatory sequences that control transcriptional expression in an expression vector and then, introduced into a microorganism. In addition to transcriptional regulatory sequences, such as promoters and enhancers, expression vectors can include translational regulatory sequences and a marker gene which is suitable for selection of cells that carry the expression vector.

The terms “overexpress”, “overexpressing”, “overexpressed” and “overexpression” refer to expression of a gene product, in particular to enhancing the expression of a gene product at a level greater than that present prior to a genetic alteration of the starting microorganism. In some embodiments, a microorganism can be genetically altered (e.g., genetically engineered) to express a gene product at an increased level relative to that produced by the starting microorganism. Genetic alteration includes, but is not limited to, altering or modifying regulatory sequences or sites associated with expression of a particular gene (e.g., by adding strong promoters, inducible promoters or multiple promoters or by removing regulatory sequences such that expression is constitutive), modifying the chromosomal location of a particular gene, altering nucleic acid sequences adjacent to a particular gene such as a ribosome binding site or transcription terminator, increasing the copy number of a particular gene, modifying proteins (e.g., regulatory proteins, suppressors, enhancers, transcriptional activators and the like) involved in transcription of a particular gene and/or translation of a particular gene product, or any other conventional means of deregulating expression of a particular gene using routine in the art (including but not limited to use of antisense nucleic acid molecules, for example, to block expression of repressor proteins). Another way to overexpress a gene product is to enhance the stability of the gene product to increase its lifetime.

References to “acids” should, unless otherwise stated or harmful toward the intended purpose of the aspect in question, also be understood to includes salts thereof or combinations of salts and acids at a suitable pH, including buffered compositions.

The terms “transformation,” “transform,” “transformed,” and derivatives thereof are meant to include any method of introducing material into a cell, including without limitation, transcription, transfection, expression, electroporation, nuclear microinjection, transduction, transfection, mediated transfection or transfection using a recombinant phage virus, incubation with calcium phosphate DNA, high velocity bombardment with DNA-coated microprojectiles, protoplast fusion and the like.

The term “cyclic hydrocarbon” includes monocyclic and multicyclic hydrocarbons.

The term “heterologous,” as used with nucleic acids, proteins, sequences, and other material, refers to nucleic acids, proteins, sequences, and other materials that do not naturally occur in a particular cell. For example, certain nucleic acids that occur naturally in a fungus to express proteins within fungus cells, may be transformed into E. coli cell, by way of not limiting example, to express the same or similar proteins within the E. coli cells Because the transformed nucleic acids do not appear naturally in the E. coli, they are heterologous nucleic acids within this particular host cell.

“DHN” means 1,8-dihydroxynaphthalene and may be referenced as “1,8-DHN.” The term “DHN” or its equivalents also includes salts of DHN.

“DHN melanin pathways” are any pathways found in nature that convert material into DHN and ultimately melanin. Typically, a DHN melanin pathway accounts for conversion of acetyl-CoA to DHN as an intermediary to the synthesis of melanin. These pathways may also be referred to as DHN expressing pathways, 1,8-DHN expressing pathways, or the shortened DHN pathway.

The term “protein” includes polypeptides, peptides, and enzymes and these terms may be used interchangeably in certain contexts.’

A “DHN melanin pathway protein,” or “protein” found in, related to, or supporting, a DHN melanin pathway includes any of the proteins in the DHN melanin pathway, including protein molecules, polypeptides, enzymes that may act upon those proteins and/or polypeptides, and the genes that express these proteins. These may also be referred to as a “DHN pathway protein or polypeptide.” Thus, a DHN melanin pathway protein may include polypeptides, enzymes, and other types of proteins that contribute to the transformation or conversion of certain molecular structures into DHN. Accordingly, when used in conjunction with a DHN melanin pathway, the term protein may be used to refer to a polyketide synthase that helps convert acetyl CoA into 1,3,6,8-tetrahydroxynapthalene (T4HN), T4HN reductase, and enzyme that helps convert T4HN into 1,3,8-trihydroxynapthalene (T3HN), and the genes or nucleic acids that express polyketide synthase, T4HN, T4HN reductase, and the like. These proteins may be referred to as “proteins related to DHN production” or DHN production proteins. The genes that express these proteins may be referred to as a gene in the DHN melanin pathway or a DHN production-related gene. A “DHN melanin pathway” or “DHN pathway” protein, polypeptide, or enzyme may include one or more of a type I polyketide synthase, a type III polyketide synthase, a T3HN reductase, a T4HN reductase, a scytalone dehydratase, a bacterial DHN (dihydroxynaphthalene) synthase A, and/or a bacterial DHN (dihydroxynaphthalene) synthase B.

As used herein, “polypeptides” includes polypeptides, proteins, peptides, fragments of polypeptides, and fusion polypeptides. In some embodiments, the fusion polypeptide includes part or all of a first polypeptide (e.g., a polyketide synthase, or DHN melanin pathway polypeptide or catalytically active fragment thereof) and may optionally include part or all of a second polypeptide (e.g., a peptide that facilitates purification or detection of the first polypeptide). By “heterologous polypeptide” is meant a polypeptide whose amino acid sequence is not identical to that of another polypeptide naturally expressed in the same host cell. In particular, a heterologous polypeptide is not identical to a wild-type nucleic acid that is found in the same host cell in nature.

As used herein, a “nucleic acid” refers to two or more deoxyribonucleotides and/or ribonucleotides in either single or double-stranded form. In some embodiments, the nucleic acid is a recombinant nucleic acid. A “recombinant nucleic acid” means a nucleic acid of interest that is free of one or more nucleic acids (e.g., genes) which, in the genome occurring in nature of the organism from which the nucleic acid of interest is derived, flank the nucleic acid of interest. The term therefore includes, for example, a recombinant DNA which is incorporated into a vector, into an autonomously replicating plasmid or virus, or into the genomic DNA of a prokaryote or eukaryote, or which exists as a separate molecule (e.g., a cDNA, a genomic DNA fragment, or a cDNA fragment produced by PCR or restriction endonuclease digestion) independent of other sequences. In various embodiments, a nucleic acid is a recombinant nucleic acid. In some embodiments, a polyketide synthase nucleic acid or other DHN melanin pathway nucleic acid is operably linked to another nucleic acid encoding all or a portion of another polypeptide such that the recombinant nucleic acid encodes a polypeptide that includes a polyketide synthase, or MVA pathway polypeptide and all or part of another polypeptide (e.g., a peptide that facilitates purification or detection of the fusion polypeptide). In some embodiments, part or all of a recombinant nucleic acid is chemically synthesized.

A “DHN pathway nucleic acid” may include one or more naturally-occurring nucleic acids such as a type I polyketide synthase nucleic acid, a type III polyketide synthase nucleic acid, a T3HN reductase nucleic acid, a T4HN reductase nucleic acid, a scytalone dehydratase nucleic acid, a bacterial DHN (dihydroxynaphthalene) synthase A nucleic acid, and/or a bacterial DHN (dihydroxynaphthalene) synthase B nucleic acid.

In some embodiments, the nucleic acid is a heterologous nucleic acid. In this context, the “heterologous nucleic acid” is meant a nucleic acid whose nucleic acid sequence is not identical to that of another nucleic acid naturally found in the same host cell.

A “vector” may be a DNA molecule used as a vehicle to artificially carry foreign genetic material into another cell, where it can be replicated and/or expressed. The term “vector” also includes any construct that is capable of delivering, and desirably expressing one or more nucleic acids of interest in a host cell. Examples of vectors include, but are not limited to, plasmids, viral vectors, DNA or RNA expression vectors, cosmids, and phage vectors. In some embodiments, the vector contains a nucleic acid under the control of an expression control sequence.

As used herein, an “expression control sequence” means a nucleic acid sequence that directs transcription or transformation of a nucleic acid of interest. An expression control sequence can be a promoter, such as a constitutive or an inducible promoter, or an enhancer. An “inducible promoter” is a promoter that is active under environmental or developmental regulation. The expression control sequence is operably linked to the nucleic acid segment to be transcribed.

As used herein throughout, the terms “inhibit,” “inhibiting,” “inhibition,” “inhibitor” or derivatives thereof do not mean completely stopping or completely preventing an activity, but rather limiting the activity or reducing the amount of activity. Similarly, the terms “inhibiting compound,” “inhibitor compounds,” “inhibition compounds,” and the like refer to compounds that limit an activity or reduce the amount of an activity. Accordingly, inhibiting the production of melanin from DHN does not mean that DHN can't or won't produce some or a lot of melanin.

The term “fuel” includes all types of fuels, including without limitation jet fuels. The term “fuel” also include fuel components or ingredients.

### EMBODIMENT EXAMPLES

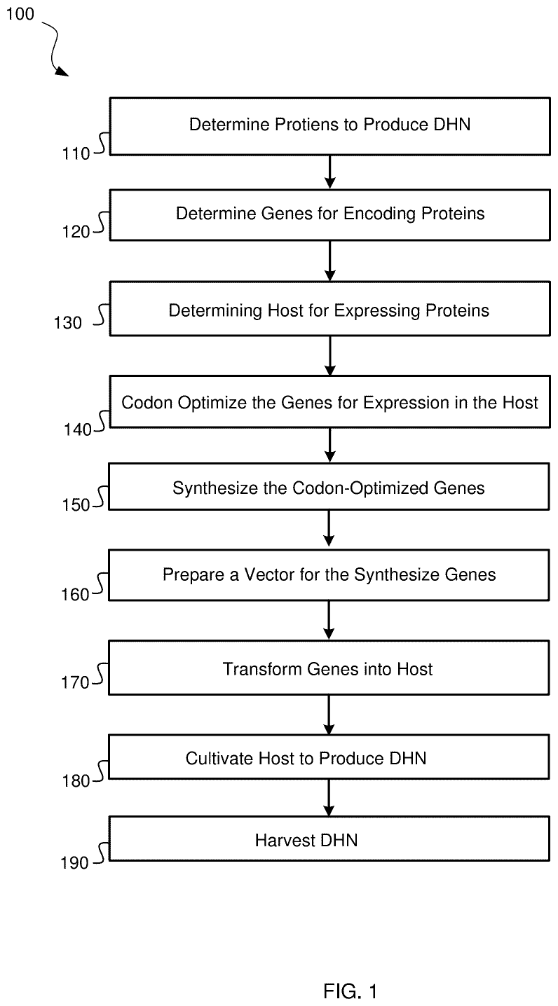

Embodiments of the present invention describe cell compositions and methods of producing DHN, which can be used to make cyclic hydrocarbons such as jet fuel in a more economically efficient and carbon-neutral way. Turning now to FIG. 1, a flow chart 100 illustrating a general scheme for producing 1,8-dihydroxynaphthalene (DHN) is shown. A first step 110 may include determining what proteins may be used to produce DHN. In one aspect, this may include finding where DHN exists in nature. In one aspect, a DHN melanin pathway may be examined. The genes that encode these proteins may then be determined 120. A host may be determined 130 or chosen in which the genes may be expressed. The genes may then be codon-optimized 140 for expression in the host. Codon optimization is based in part on the fact that amino acids may be represented by two or more codons, allowing for options in how the codon is written while still expressing the same gene. Thus, in one embodiment, the codons of the one or more of the recombinant genes used to produce DHN may be optimized for better performance in the host cell.

The codon-optimized genes may then be synthesized 150 and placed 160 in a prepared vector. The codon-optimized genes and accompanying vector may then be transformed 170 into the host. The host cells may be cultivated 180 to produce DHN, which may then be harvested 190.

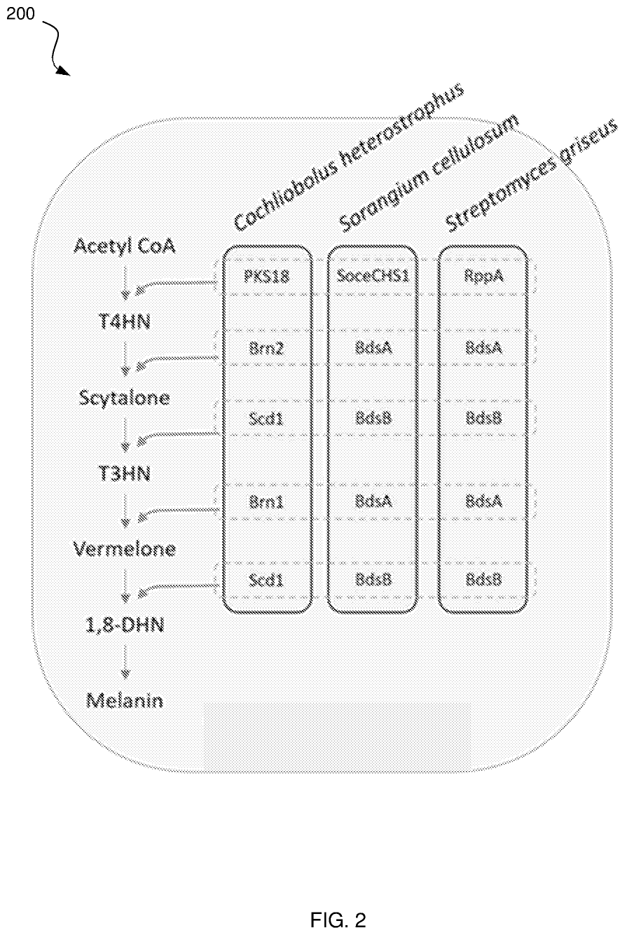

Turning now to FIG. 2, the determination of proteins used to produce DHN and the genes that encode these proteins may be determined by looking at various organisms in nature. The DHN expressing pathway may be part of the melanin pathway. Thus, DHN melanin pathways are good places to look for proteins used in the production of DHN. Several species of fungi and bacteria have DHN melanin pathways. In one embodiment, proteins and nucleic acids in the DHN melanin pathways of Cochliobolus heterostrophus, a fungus, and the bacterium Sorangium cellulosum and Streptomyces griseus were chosen.

As can be seen in the figure, the DHN melanin pathway begins with acetyl-CoA. A polyketide synthase (PKS) protein converts the acetyl-CoA to T4HN. A nucleic acid in the form of a gene expresses the polyketide synthase. In one embodiment, this gene may be PKS18 from a Cochliobolus heterostrophus. In another embodiment, the gene may be SoceCHS1 from Sorangium cellulosum. In yet other embodiments, this gene may be RppA from Streptomyces griseus. Other genes from other sources may be used to express a polyketide synthase for converting Acetyl CoA to T4HN.

T4HN may then be converted into Scytalone by a T4HN reductase. The T4HN reductase may be expressed by the Brn2 gene from a Cochliobolus heterostrophus, the BdsA gene from a Sorangium cellulosum and/or a Streptomyces griseus. Scytalone may then be converted into T3HN by a scytalone dehydratase. The scytalone dehydratase may be expressed from the Scd1 gene of Cochliobolus heterostrophus and/or the BdsB genes of Sorangium cellulosum or Streptomyces griseus. T3HN may then be converted into Vermelone by a 1,3,8-trihydroxynapthalene (T3HN) reductase, which may be expressed the Brn1 gene from a Cochliobolus heterostrophus, and/or the BdsA gene from a Sorangium cellulosum or a Streptomyces griseus. Ultimately, vermelone may be converted into DHN by a scytalone dehydratase. The scytalone dehydratase may be the same scytalone dehydratase described above that is expressed from the Scd1 gene of Cochliobolus heterostrophus and/or the BdsB genes of Sorangium cellulosum or Streptomyces griseus.

A series of conversions occur by multiple enzymes. The first series of enzymes are expressed by the genes PKS18, Brn1, Brn2, and Scd1 found in Cochliobolus heterostrophus. The Scd1 gene encodes an enzyme that has multiple duties along the Cochliobolus heterostrophus DHN pathway. Another series of enzymes are expressed by the genes SoceCHS1, BdsA, and BdsB, found in Sorangium cellulosum. Here again, the enzymes encoded by BdsA and BdsB serve multiple functions along the Sorangium cellulosum DHN pathway. The RppA enzyme in Streptomyces griseus strain was reported to have a function similar to PKS18, thus another enzyme set was designed by the enzymes encoded by the genes RppA, BdsA, BdsB, BdsA, and BdsB.

### Exemplary Nucleic Acids

It will be appreciated that the genes discussed herein throughout are nucleic acids. Accordingly, as discussed above, various DHN nucleic acids can be used in the compositions and methods of the invention. In particular embodiments, the nucleic acid includes a segment of, or the entire nucleic acid sequence of, any naturally-occurring polyketide synthase and/or or any DHN pathway nucleic acid. In some embodiments, the nucleic acid includes at least or about 50, 100, 150, 200, 300, 400, 500, 600, 700, 800, or more contiguous nucleotides from a naturally-occurring type I polyketide synthase nucleic acid, type III polyketide synthase nucleic acid, T3HN reductase nucleic acid, T4HN reductase nucleic acid, scytalone dehydratase nucleic acid, bacterial DHN (dihydroxynaphthalene) synthase A nucleic acid, and/or bacterial DHN (dihydroxynaphthalene) synthase B nucleic acid.

In some embodiments, the nucleic acid has one or more mutations compared to the sequence of a wild-type (i.e., a sequence occurring in nature) nucleic acids described above. In some embodiments, the nucleic acid has one or more mutations that increase the transcription or translation of a DHN pathway nucleic acid. In some embodiments, the nucleic acid is a degenerate variant of any nucleic acid encoding a DHN pathway polypeptide. Exemplary nucleic acids include nucleic acids that encode a polypeptide, fragment of a polypeptide, peptide, or fusion polypeptide that has at least one activity of a polyketide synthase polypeptide or a DHN pathway polypeptide. In certain embodiments, exemplary nucleic acids include naturally-occurring nucleic acids from any of the source organisms described herein as well as mutant nucleic acids derived from any of the source organisms described herein

In some embodiments, the nucleic acids may be placed in appropriate expression cassettes for selected species, such as species of bacteria or fungi that do not actively convert DHN into melanin. These nucleic acids may be taken from various microbes that produce melanin, related compounds, or naphthalene derivatives in nature. One or more or all of the nucleic acids associated with any of the sources described herein throughout may be placed in an appropriate expression cassette. The expression cassettes may be configured by source. For example, the fungal species Cochliobolus heterostrophus contains a DHN melanin pathway with the following nucleic acids in the form of genes, each listed with the protein enzyme they produce and the number of base pairs (bp) in the gene codon:
PKS18: polyketide synthase, 6468 bp
BRN1: 1,3,8-trihydroxynapthalene (T3HN) reductase, 801 bp
BRN2: 1,3,6,8-tetrahydroxynapthalene (T4HN) reductase, 804 bp
SCD1: scytalone dehydratase, 555 bp
   One or more or all of these nucleic acids may be arranged in one or more expression cassettes. The DHN pathway for the bacterial species Sorangium cellulosum contains the following nucleic acids in the form of genes, each listed with the protein enzyme they produce and the number of base pairs (bp) in the gene codon:
soceCHS1: a type III polyketide synthase, 1089 bp
BdsA: bacterial DHN (dihydroxynaphthalene) synthase A, 960 bp
BdsB: bacterial DHN (dihydroxynaphthalene) synthase B, 330 bp
   One or more or all of these nucleic acids may be arranged in one or more expression cassettes. The DHN pathway for the bacterial species Streptomyces griseus contains the following nucleic acids in the form of genes, each listed with the protein enzyme they produce and the number of base pairs (bp) in the gene codon:
RppA: a type III polyketide synthase, 1116 bp
   This nucleic acid may be arranged in one or more expression cassettes with nucleic acid expressing non-polyketide synthases such as those described above. Other DHN pathway nucleic acids, including without limitation the gene PKSsw, may also be used and configured within appropriate expression cassettes.

When these nucleic acids are expressed in host cells described below, embodiments of cells may include a nucleic add encoding a polyketide synthase polypeptide and one or more nucleic acids encoding one or more proteins used in the production of DHN. These proteins may be one or more proteins in a DHN melanin pathway. In certain embodiments, the cells include nucleic acids encoding one or more proteins in one or more DHN melanin pathways found in the fungal species, Cochliobolus heterostrophus, the bacterial species Sorangium cellulosum, and/or the bacterial species Streptomyces griseus. In one embodiment, the cells may include one or more nucleic acids encoding the polypeptides of an entire DHN melanin pathway. Accordingly, cells of embodiments of the present invention may include a nucleic acid encoding a polyketide synthase polypeptide that includes one or more of PKS18, soceCHS1, RppA, and PKSwd. In other embodiments, the cells may include one or more nucleic acids encoding one or more of BRN1, BRN2, SCD1, BdsA, and BdsB In one particular embodiment cells contain one or more nucleic acids encoding a PKS18 and either i) Brn1, Brn2, and Scd1 or ii) BdsA and BdsB.

Exemplary Proteins and/or Polypeptides

Exemplary proteins and/or polypeptides include the proteins and/or polypeptides expressed by the nucleic acids and genes discussed herein throughout. As defined above, in many contexts, “nucleic acids” and “genes” are used synonymously, as are “proteins,” “polypeptides,” and “enzymes.” As the proteins are expressed in a host cell, cells of embodiments of the present invention will include those proteins, in addition to the nucleic acids that express them. Accordingly, in one embodiment, where nucleic acids express proteins in the form of polypeptide enzymes found in the DHN melanin pathway of the fungal species Cochliobolus heterostrophus, the cells may include a polyketide synthase polypeptide that is a fungal polyketide synthase polypeptide. Where the nucleic acids express proteins in the form of polypeptide enzymes found in the DHN melanin pathways of either Sorangium cellulosum or Streptomyces griseus, the polyketide synthase polypeptide expressed is a bacterial polyketide synthase polypeptide. In certain embodiments, the cells contain type I polyketide synthases. In other embodiments, the cells contain type I polyketide synthases.

In one embodiment the cells include nucleic acids encoding one or more of 1,3,8-trihydroxynapthalene (T3HN) reductase, 1,3,6,8-tetrahydroxynapthalene (T4HN) reductase, scytalone dehydratase, bacterial DHN (dihydroxynaphthalene) synthase A, and bacterial DHN (dihydroxynaphthalene) synthase B. Accordingly, in certain embodiments, the cells include one or more of 1,3,8-trihydroxynapthalene (T3HN) reductase, 1,3,6,8-tetrahydroxynapthalene (T4HN) reductase, scytalone dehydratase, bacterial DHN (dihydroxynaphthalene) synthase A, and bacterial DHN (dihydroxynaphthalene) synthase B.

In other embodiments, proteins and corresponding genes and/or nucleic acids from other DHN melanin pathways may be used. DHN melanin pathways from other organisms such as yeasts, bacteria, and other fungi can be sources for genes other than the specific genes already mentioned above. For example, the DHN-melanin biosynthetic pathway which produces fungal eumelanin may include a polyketide synthase (pks), α/β hydrolase (ayg1), scytalone dehydratase (arp1), 1,3,6,8-tetrahydroxynaphthalene reductase (arp2), multicopper oxidases (abr1), and ferroxidase (abr1). Polyketide synthase and the α/β hydrolase are essential for melanin production in many black fungi. Various pks genes and ayg1 genes that are found in black fungi and homologues thereof and of genes mentioned above, may be in a variety of other organisms.

Also, by way of example, the black yeast species Phaeococcomyces produces melanin via the DHN melanin pathway, and has a complete set of genes capable of converting nutrients such as sugars into DHN on the way to melanin production. One or more of the genes may be used instead of the aforementioned genes. Further, other hydroxynaphthalene reductase genes may be used such as the gene arp2 from the organism Neosartorya fumigata. In other embodiments, polyketide synthase genes other than the PKS18 may be used. For example, a gene (WdPKS1) that encodes a putative polyketide synthase (WdPks1p) of Wangiella dermatitidis that is involved in dihydroxynaphthalene melanin biosynthesis and virulence in Wangiella (Exophiala) dermatitidis. The WdPks1p consisted of 2,177 amino acids and showed significant similarities with other polyketide synthases, but particularly those encoded by the pks gene of C. lagenarium, the alb1 gene of A. fumigatus, and the wA gene of Aspergillus nidulans. Any of these PKS genes may be considered as a replacement in whole or part for or in addition to PKS18. Each such gene may be codon optimized for expression in a particular host.

In another embodiment, one or more genes from Neosartorya fumigata (strain ATCC MYA-4609/Af293/CBS 101355/FGSC A1100) (Aspergillus fumigatus) may be used. The first step of the DHN-melanin pathway for this organism is the production of the heptaketide naphtopyrone YWA1 by the polyketide synthase alb1 though condensation of acetyl-CoA with malonyl-CoA. The naphtopyrone YWA1 is then converted to the pentaketide 1,3,6,8-tetrahydroxynaphthalene (1,3,6,8-THN) by the heptaketide hydrolyase ayg1 though chain-length shortening. 1,3,6,8-THN is substrate of the hydroxynaphthalene reductase arp2 to yield scytalone. The scytalone dehydratase arp1 then reduces scytalone to 1,3,8-THN. 1,3,8-THN is also substrate of the hydroxynaphthalene reductase arp2 to yield vermelone. Vermelone is further converted by the multicopper oxidase abr1 to 1,8-DHN. Finally, the laccase abr2 transforms 1,8-DHN to DHN-melanin, although it may be desirous for this gene to be absent in order to preserve the DHN, or a suitable laccase inhibitor could be used.

In another aspect, a type III polyketide can, instead of or in addition to polyketides from other genes and in addition to other host cells discussed herein, be expressed from a yeast or other organisms using the methods and materials described in WO/2017/160801, “Compositions and Methods for Type III Polyketide Production in Oleaginous Yeast Species,” issued Sep. 21, 2017 to H. Alper and M. Kelly, relevant portions hereby incorporated by reference.

In one embodiment, the following enzymes and genes used for melanin production in Aspergillus fumigatus AFGRD105 may be used:

Enzyme Gene
Polyketide synthase alb1
Polyketide shortening ayg1
Scytalone dehydratase arp1
Hydroxynaphthalene reductase arp2
Vermelone dehydratase abr1
Oxidase abr2

In certain embodiments, genes associated with products or substrates related to dihydroxynaphthalene may be used, including, without limitation, nsaC from Sphingobium xenophagum, nahC from Pseudomonas putida (Arthrobacter siderocapsulatus), alb1 pksP from Neosartorya fumigata (strain ATCC MYA-4609/Af293/CBS 101355/FGSC A1100) (Aspergillus fumigatus), Scytalone dehydratase gene arp1 from Neosartorya fumigata (strain ATCC MYA-4609/Af293/CBS 101355/FGSC A1100) (Aspergillus fumigatus), Scytalone dehydratase SDH1 MGG_05059 from Magnaporthe oryzae (strain 70-15/ATCC MYA-4617/FGSC 8958) (Rice blast fungus) (Pyricularia oryzae), hydroxynaphthalene reductase arp2 or arp2 AFUA_2G17560 of Neosartorya fumigata (strain ATCC MYA-4609/Af293/CBS 101355/FGSC A1100) (Aspergillus fumigatus), Scytalone dehydratase arp1 or arp1 AFUA 2G17580 from Neosartorya fumigata (strain ATCC MYA-4609/Af293/CBS 101355/FGSC A1100) (Aspergillus fumigatus), Scytalone dehydratase SDH1 MGG_05059 from Magnaporthe oryzae (strain 70-15/ATCC MYA-4617/FGSC 8958) (Rice blast fungus) (Pyricularia oryzae); hydroxynaphthalene reductase arp2 or arp2 AFUA_2 G17560 from Neosartorya fumigata (strain ATCC MYA-4609/Af293/CBS 101355/FGSC A1100) (Aspergillus fumigatus); scytalone dehydratase SCD1 Cob_03011/Cob_v008979 from Colletotrichum orbiculare (strain 104-T/ATCC 96160/CBS 514.97/LARS 414/MAFF 240422) (Cucumber anthracnose fungus) (Colletotrichum lagenarium); Tetrahydroxynaphthalene reductase PfmaG PFICI_07103 from Pestalotiopsis fici (strain W106-1/CGMCC3.15140), and the like.

### Exemplary Host Cells

A variety of host cells may be used to express polyketide synthase, or other DHN pathway polypeptides to produce DHN in the methods of the claimed invention. In one embodiment, the cells may include at least one cell chosen from gram-positive bacterial cells, gram-negative bacterial cells, fungal cells, filamentous fungal cells, algal cells and yeast cells. In embodiments where the cells include one or more of a gram-positive bacterial cell and a gram-negative bacterial cell, the cells may include one or more of Escherichia coli, Pseudomonas puticia, Pantoae citrea, Bacillus subtilis, Bacillus licheniformis, Bacillus lentus, Bacillus brevis, Bacillus stearothermophilus, Bacillus alkalophilus, Bacillus amyloliquefaciens, Bacillus clausii, Bacillus halodurans, Bacillus megaterium, Bacillus coagulans, Bacillus circulans, Bacillus lautus, Bacillus thuringiensis, Streptomyces lividans, Streptomyces coelicolor, Streptomyces griseus, Pseudomonas sp., and Pseudomonas alcaligenes cells. In one embodiment, the cells include at least one of Escherichia coli cells and Pseudomonas putida cells.

In one embodiment, the cells include host cells that are fungal cells. The cells may include one or more of Aspergillus sp., yeast, Trichoderma sp., Yarrowia sp., Saccharomyces sp., Schizosaccharomyces sp., Pichia sp., Candida sp., Aspergillus oryzae, Aspergillus niger, Saccharomyces cerevisiae, Schizosaccharomyces pombe, Trichoderma reesei, Humicola insolens, Humicola lanuginose, Humicola grisea, Candida lucknowense, Aspergillus sojae, Aspergillus japonicus, Aspergillus nidulans, Aspergillus aculeatus, Aspergillus awamori, Fusarium roseum, Fusarium graminum, Fusarium cerealis, Fusarium oxysporuim, F. venenatum, Neurospora crassa, Mucor miehei, Trichoderma viride, Fusarium oxysporum, and Fusarium solani cells.

In one embodiment, the cells include an algal cell. The cells may include one or more of green algae, red algae, glaucophytes, chlorarachniophytes, euglenids, chromista, and dinoflagellates.

Where any of the nucleic acids described herein throughout, and/or the proteins expressed by those nucleic acids, are not found naturally in any of the host cells described herein throughout, the nucleic acids and proteins expressed thereby are heterologous nucleic acids and heterologous proteins. Indeed, in one embodiment, the nucleic acid encoding a polyketide synthase polypeptide is a heterologous nucleic acid. Additionally, the nucleic acids encoding other polypetides, proteins, peptides or enzymes may be heterologous nucleic acids. In one embodiment, the nucleic acids described herein throughout may be a copy of an endogenous nucleic acid. In some embodiments, analogs of some of the synthases, reductases, dehydratases and the like may have overlap in endogenous enzymes, such that a copy of such endogenous nucleic acid may be used in, or be present in the cells of the embodiments described herein.

In one embodiment, the genes and/or nucleic acids described herein may be codon-optimized for expression into the host cells into which they will be transformed. In one embodiment, the host cells include E. coli and P. putida cells. Nucleic acids that express enzyme sequences for certain DHN melanin pathways may be obtained from various databases, including without limitation, the GenBank database. These nucleic acids in the form of genes were codon-optimized for E. coli and P. putida. Thus, in one embodiment, the cells may include one or more of a nucleotide sequence of a polyketide synthase gene codon-optimized for expression in E. coli, a nucleotide sequence of a polyketide synthase gene codon-optimized for expression in P. Putida, a nucleotide sequence of a 1,3,8-trihydroxynapthalene (T3HN) reductase gene codon-optimized for expression in E. coli, a nucleotide sequence of a 1,3,8-trihydroxynapthalene (T3HN) reductase gene codon-optimized for expression in P. putida, nucleotide sequence of a 1,3,6,8-tetrahydroxynapthalene (T4HN) reductase gene codon-optimized for expression in E. coli, a nucleotide sequence of a 1,3,6,8-tetrahydroxynapthalene (T4HN) reductase gene codon-optimized for expression in P. putida, a nucleotide sequence of a scytalone dehydratase gene codon-optimized for expression in E. coli, a nucleotide sequence of a scytalone dehydratase gene codon-optimized for expression in P. putida, a nucleotide sequence of a bacterial DHN (dihydroxynaphthalene) synthase A gene codon-optimized for expression in E. coli, a nucleotide sequence of a bacterial DHN (dihydroxynaphthalene) synthase A gene codon-optimized for expression in P. putida, a nucleotide sequence of a bacterial DHN (dihydroxynaphthalene) synthase B gene codon-optimized for expression in E. coli, and a nucleotide sequence of a bacterial DHN (dihydroxynaphthalene) synthase B gene codon-optimized for expression in P. putida.

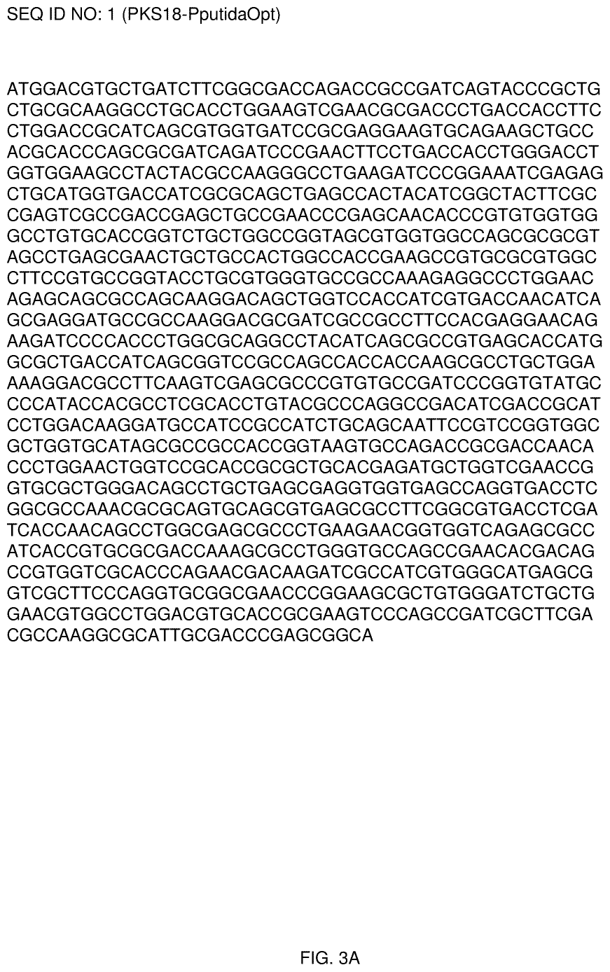

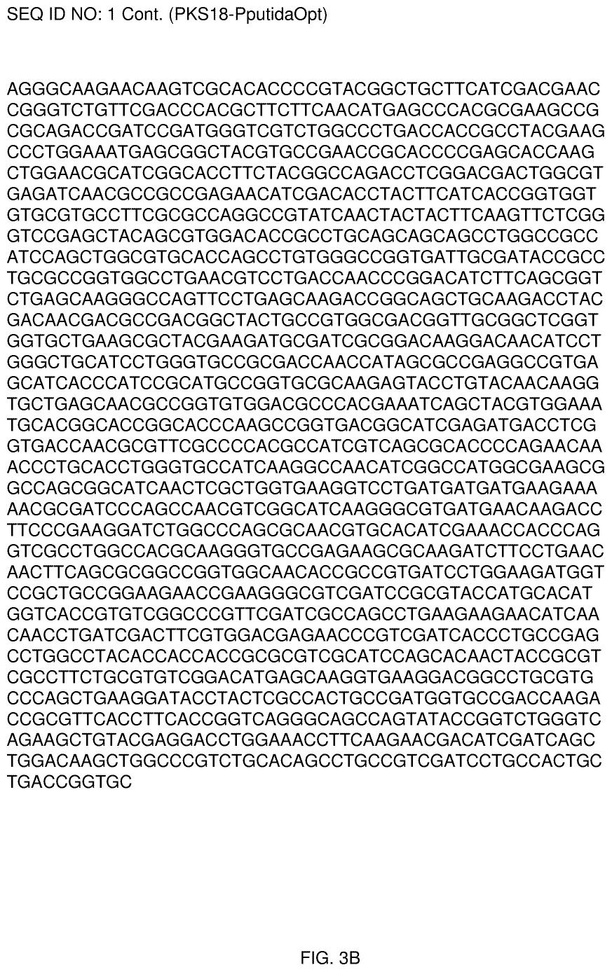

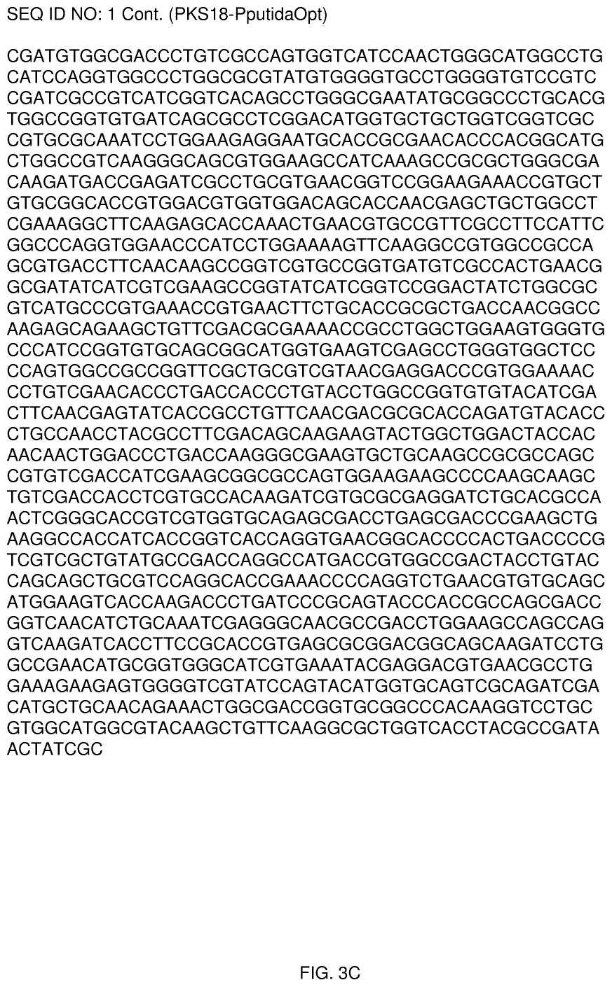

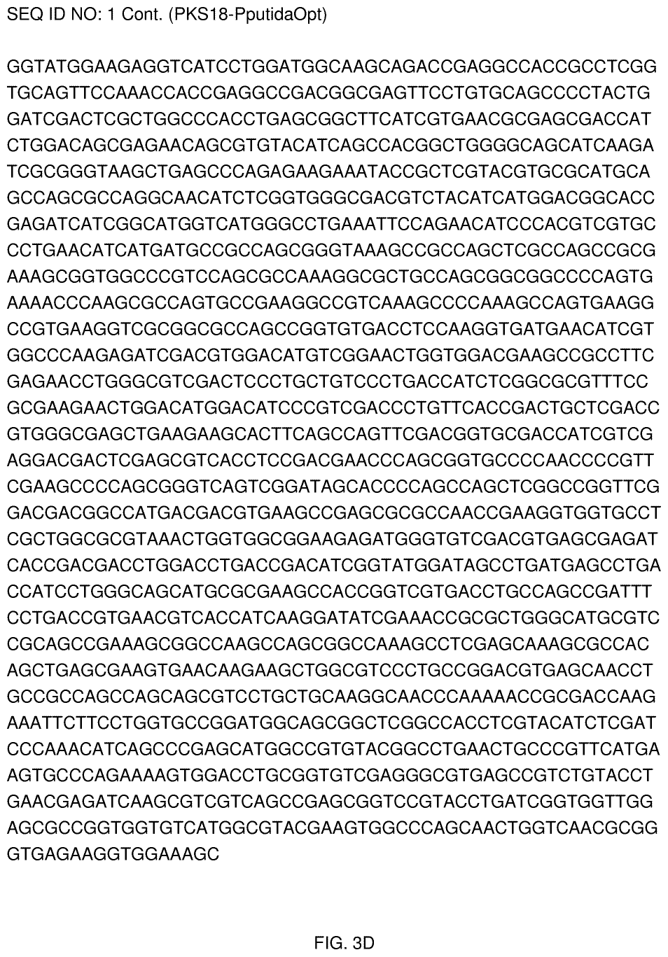

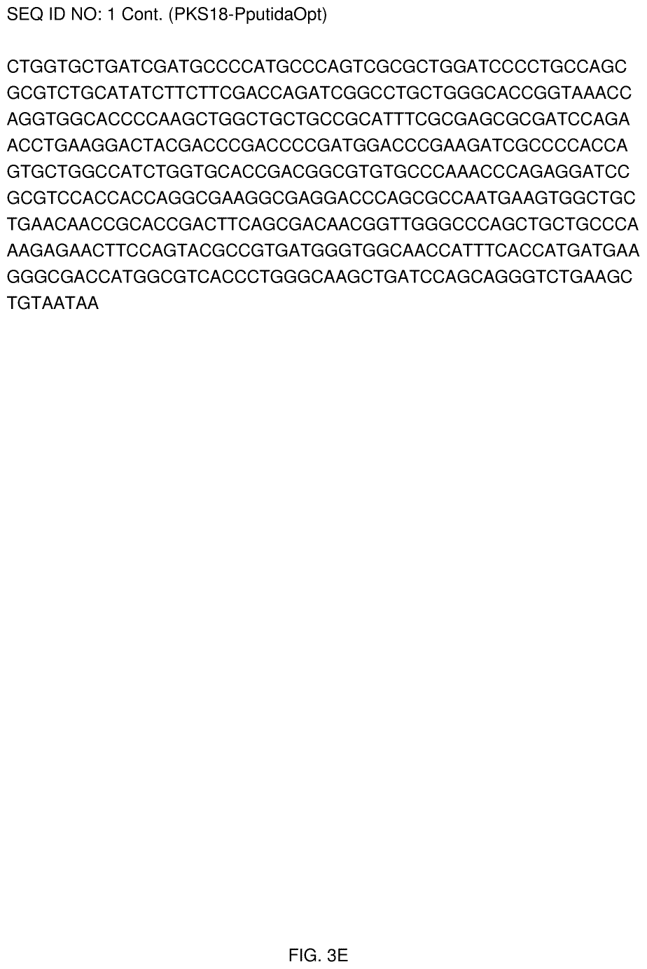

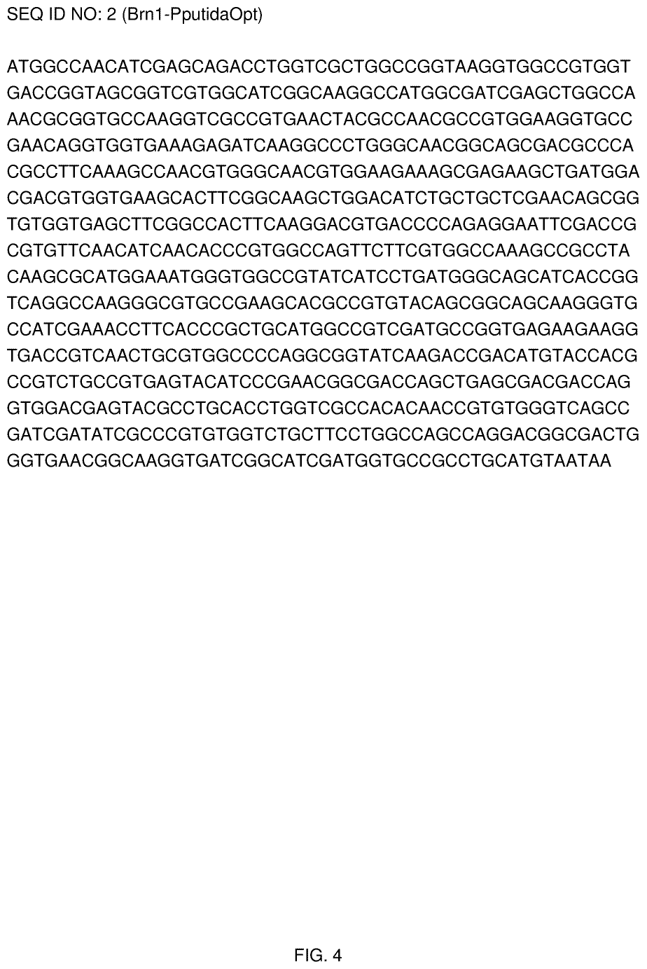

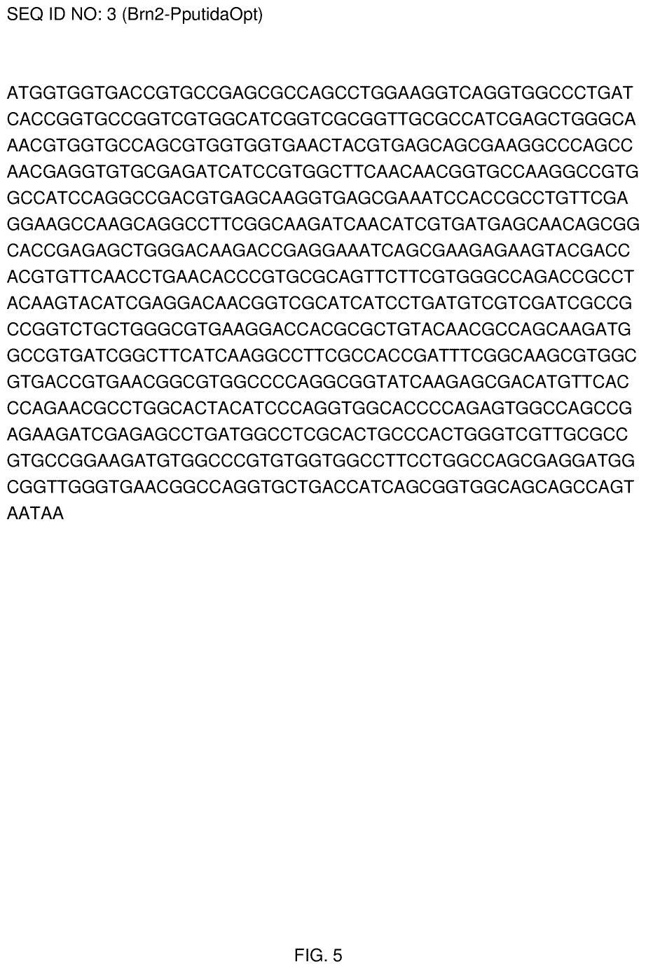

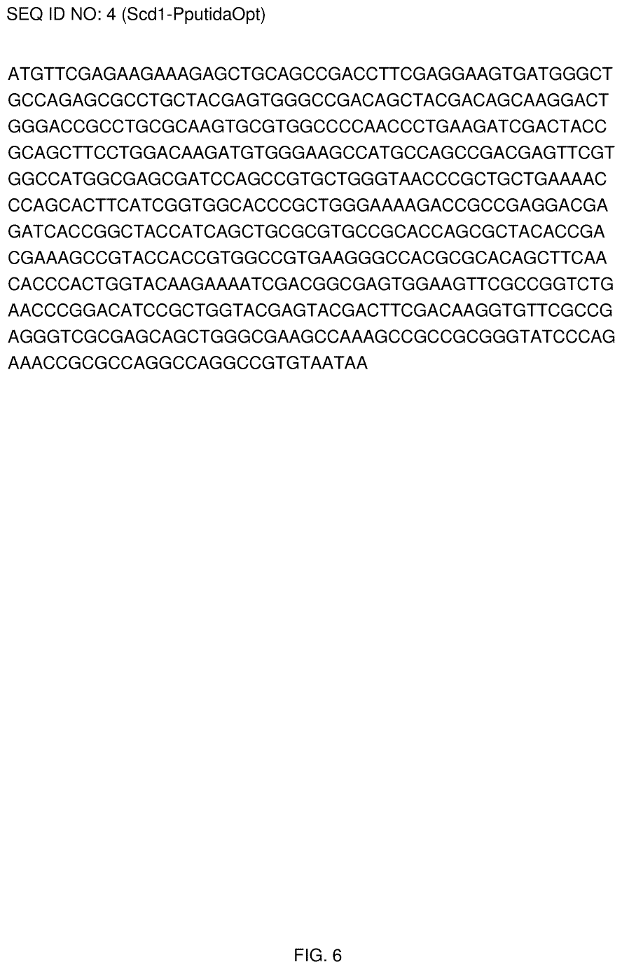

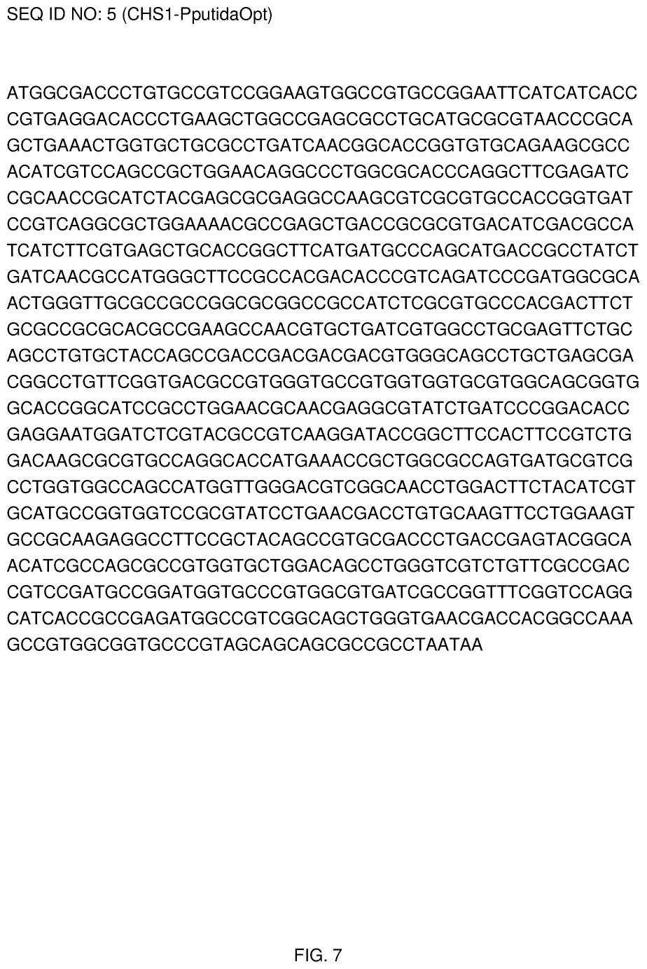

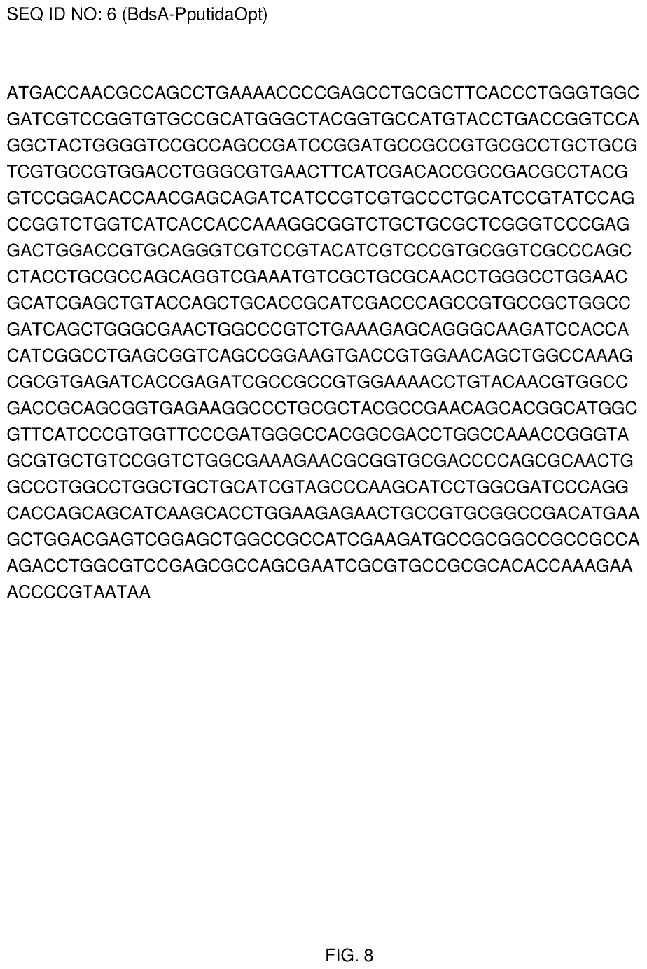

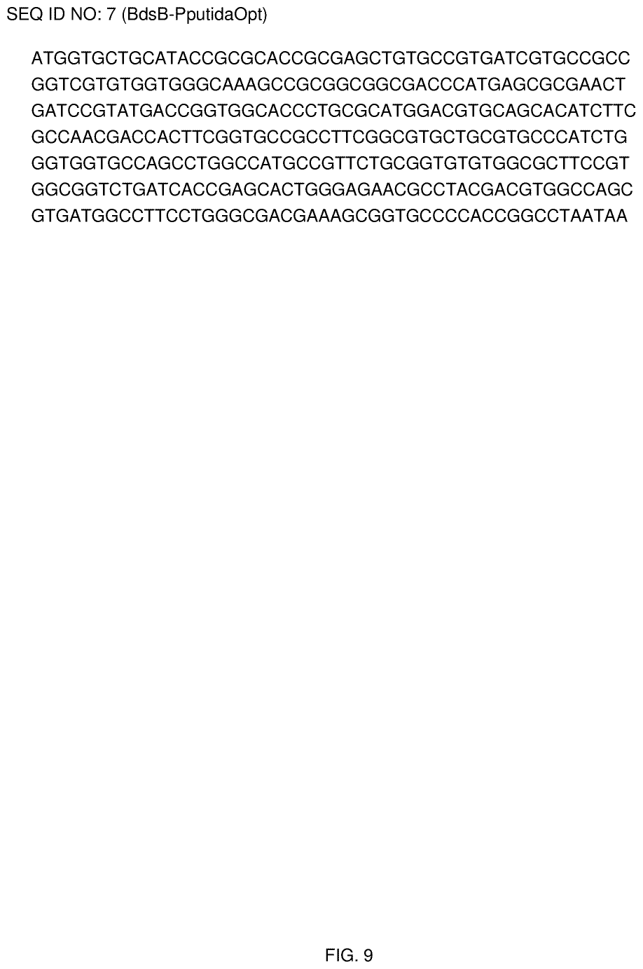

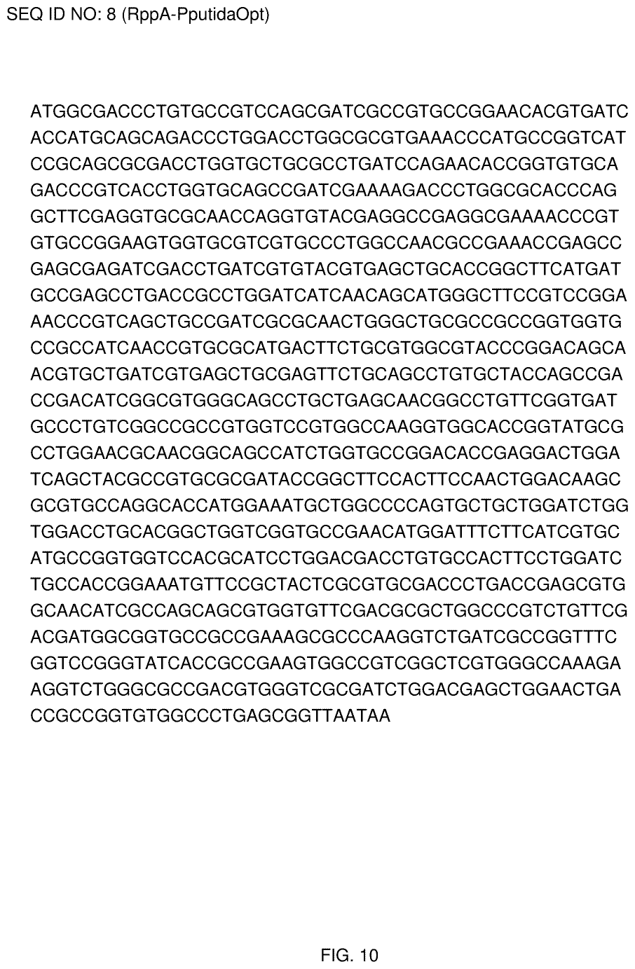

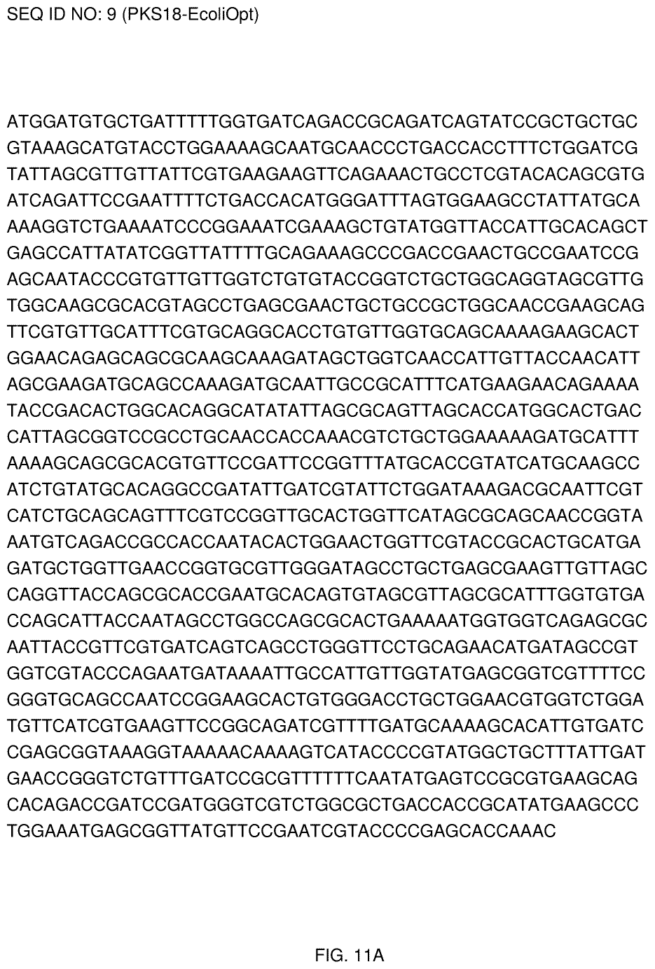

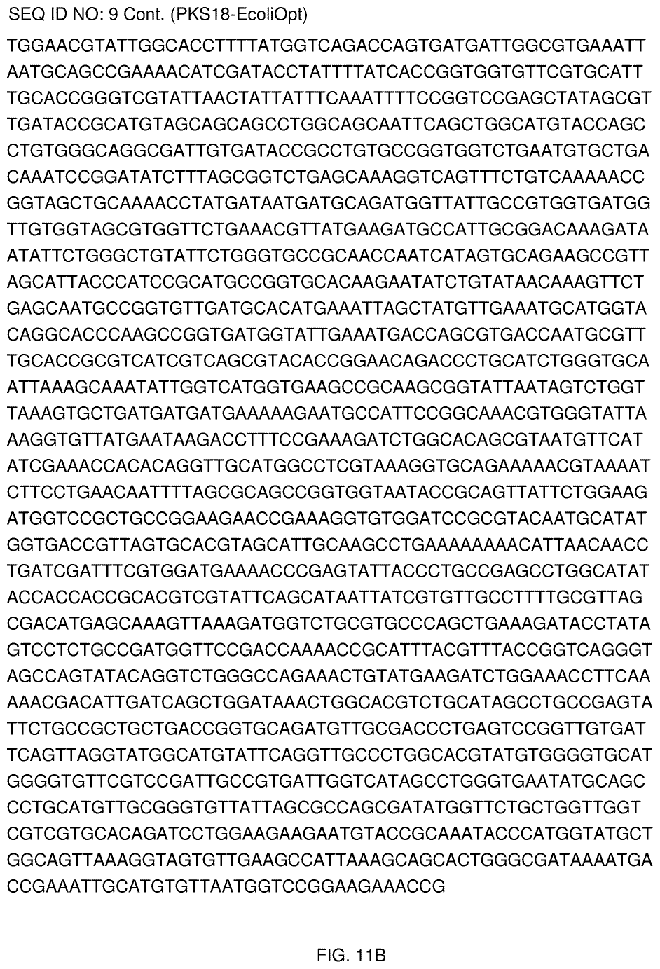

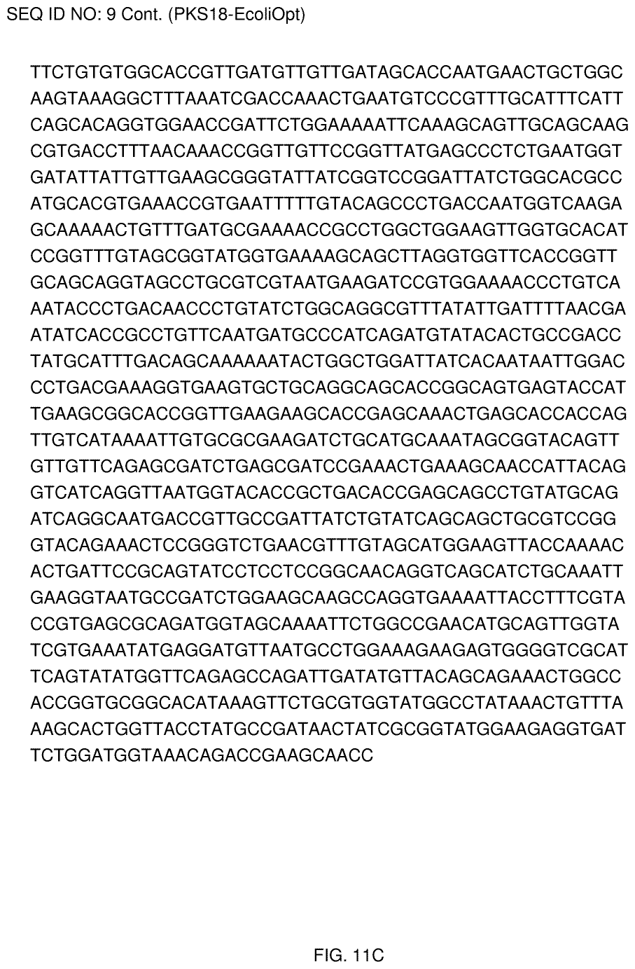

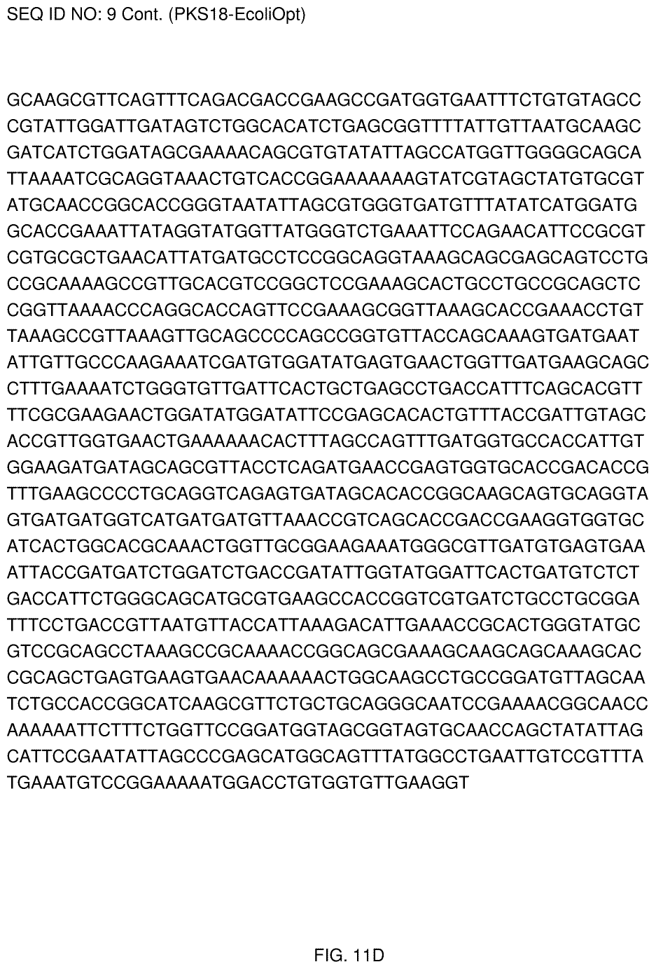

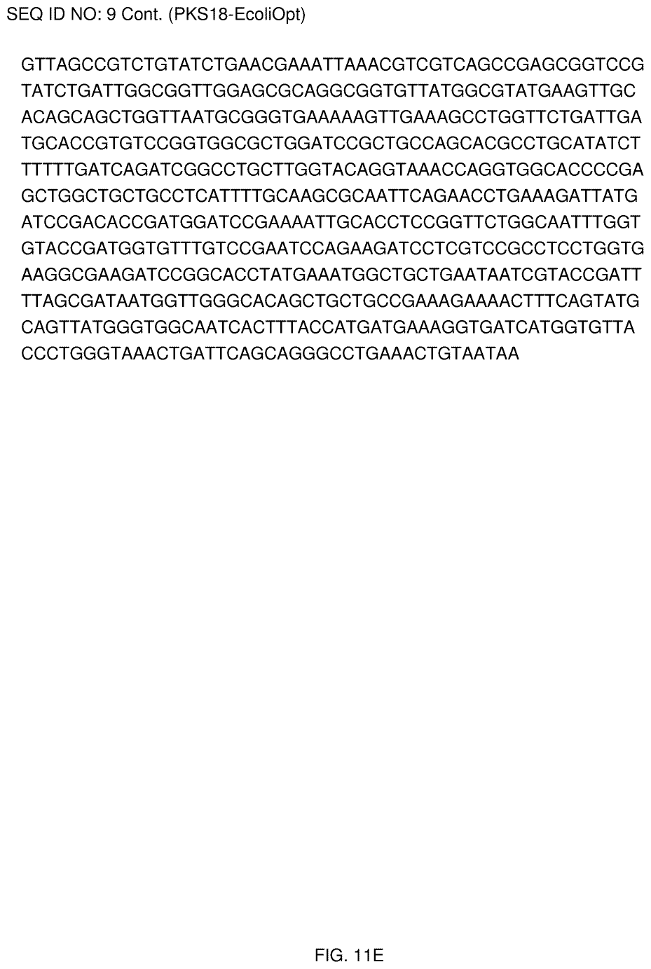

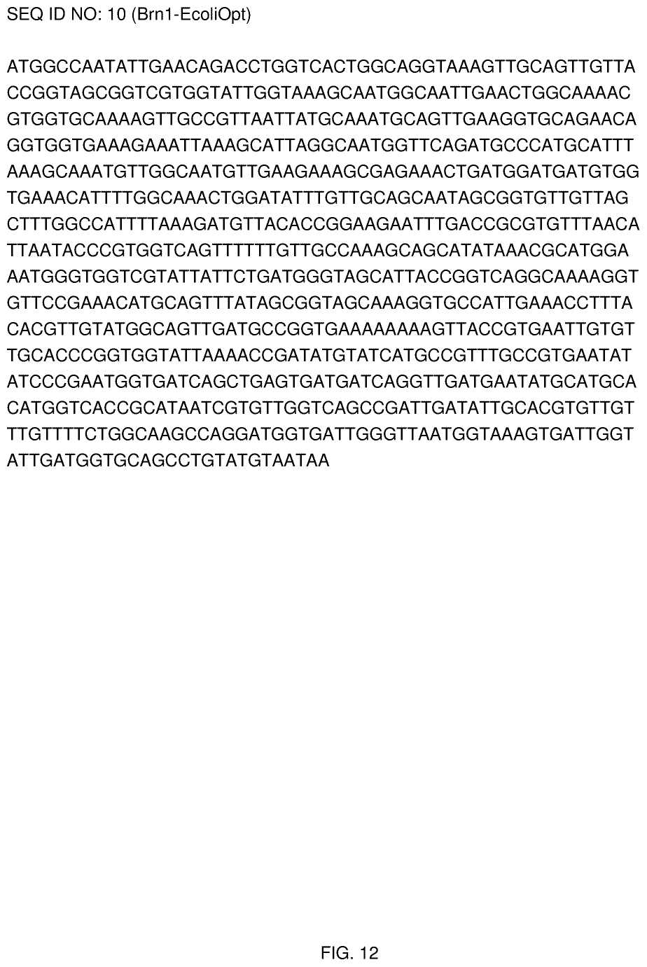

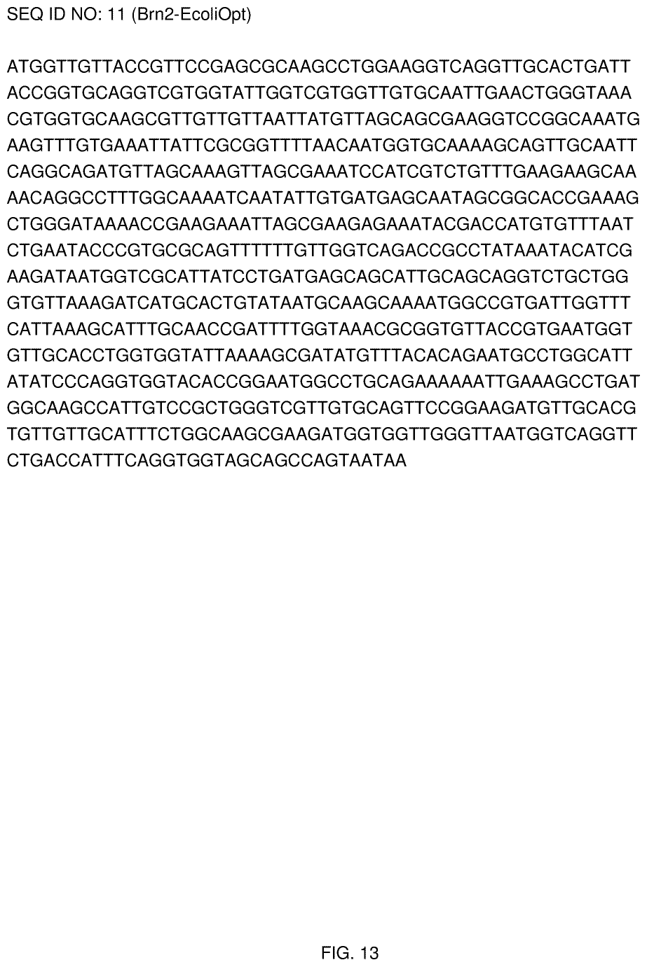

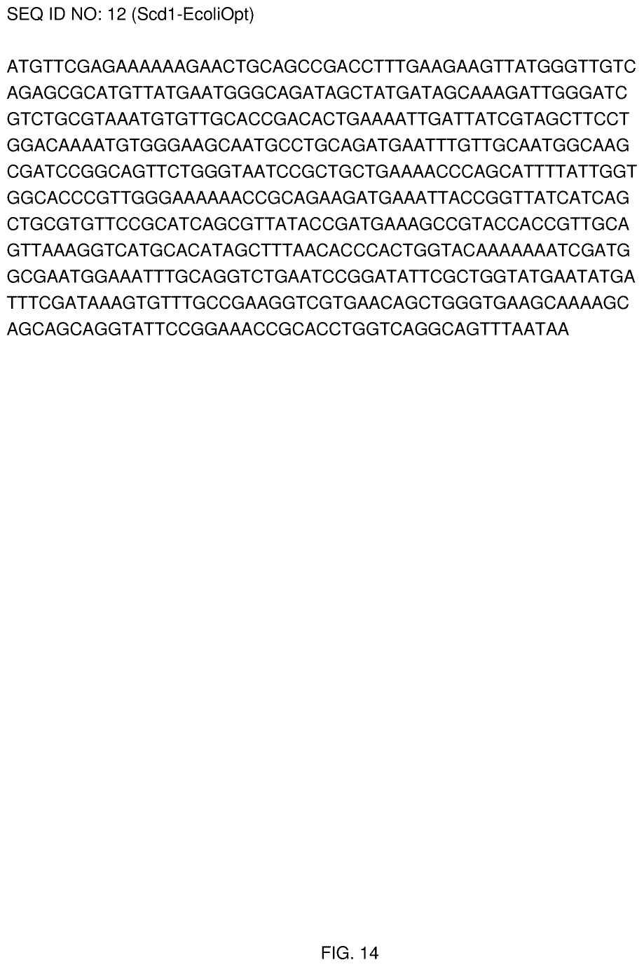

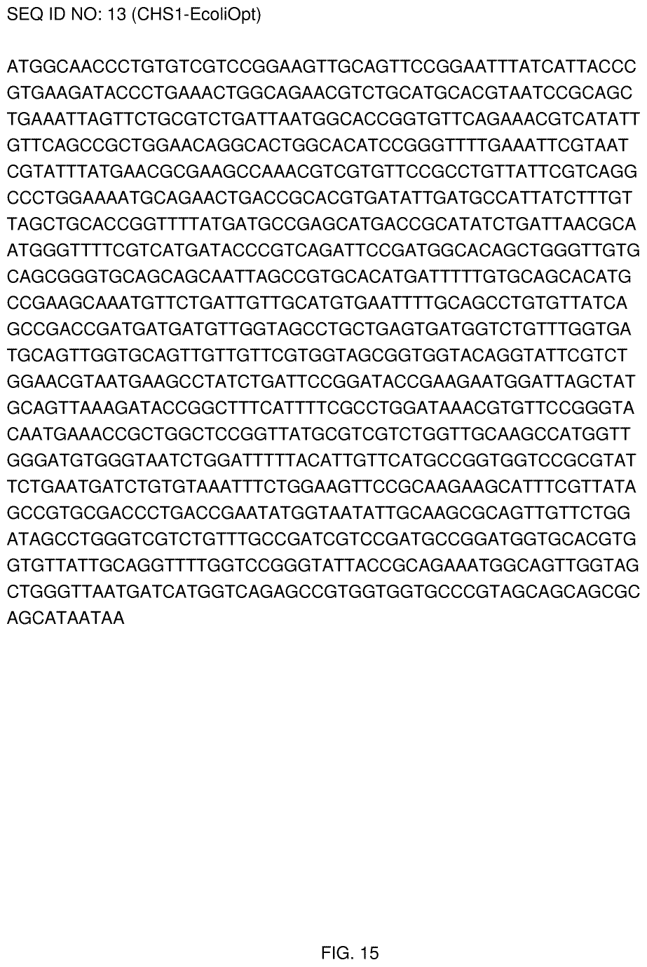

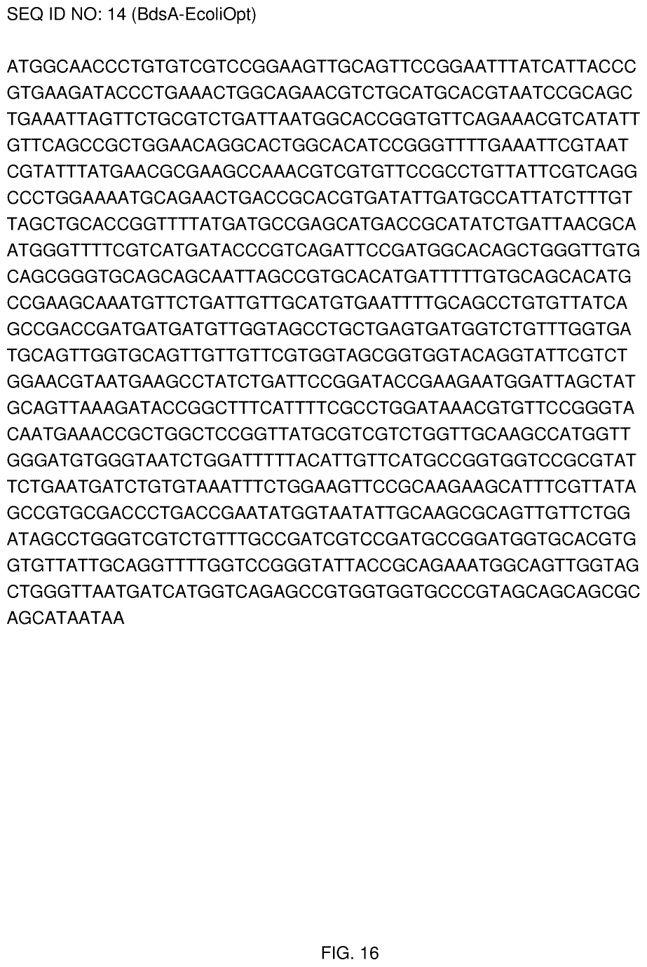

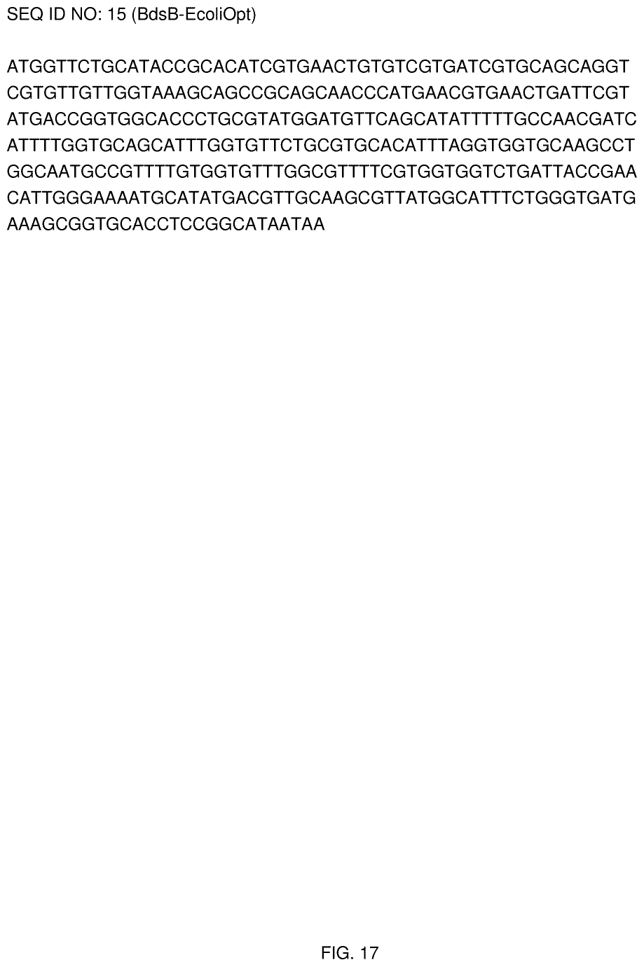

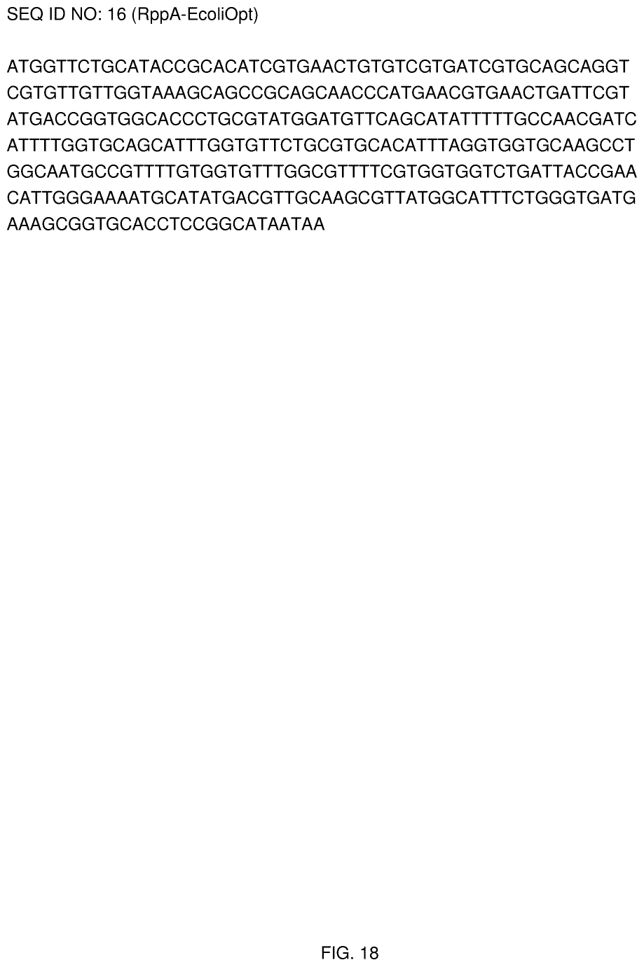

Turning now to FIGS. 3 through 18, the nucleotide sequences for these codon-optimized genes are shown. Specifically, FIGS. 3A-3E show the nucleotide sequence of PKS18, a polyketide synthase gene codon-optimized for expression in Pseudomonas putida (P. putida) (SEQ ID NO:1). FIG. 4 is the nucleotide sequence of Brn1, a 1,3,8-trihydroxynapthalene (T3HN) reductase gene codon-optimized for expression in P. putida (SEQ ID NO: 2). FIG. 5 is the nucleotide sequence of a Brn2, 1,3,6,8-tetrahydroxynapthalene (T4HN) reductase gene codon-optimized for expression in P. putida (SEQ ID NO: 3). FIG. 6 is the nucleotide sequence of Scd1, a scytalone dehydratase gene codon-optimized for expression in P. putida (SEQ ID NO: 4). FIG. 7 is the nucleotide sequence of CHS1, a polyketide synthase gene codon-optimized for expression in P. putida (SEQ ID NO: 5). FIG. 8 is the nucleotide sequence of BdsA, a bacterial DHN synthase A gene codon-optimized for expression in P. putida (SEQ ID NO: 6). FIG. 9 is the nucleotide sequence of BdsB, a bacterial DHN synthase B gene codon-optimized for expression in P. putida (SEQ ID NO: 7). FIG. 10 is the nucleotide sequence of RppA, a polyketide synthase gene codon-optimized for expression in P. putida (SEQ ID NO: 8). FIGS. 11A-11E show the nucleotide sequence of PKS18, a polyketide synthase gene codon-optimized for expression in Escherichia coli (E. coli) (SEQ ID NO:9). FIG. 12 is the nucleotide sequence of Brn1, a 1,3,8-trihydroxynapthalene (T3HN) reductase gene codon-optimized for expression in E. coli (SEQ ID NO: 10). FIG. 13 is the nucleotide sequence of a Brn2, 1,3,6,8-tetrahydroxynapthalene (T4HN) reductase gene codon-optimized for expression in E. coli (SEQ ID NO: 11). FIG. 14 is the nucleotide sequence of Scd1, a scytalone dehydratase gene codon-optimized for expression in E. coli (SEQ ID NO: 12). FIG. 15 is the nucleotide sequence of CHS1, a polyketide synthase gene codon-optimized for expression in E. coli (SEQ ID NO: 13). FIG. 16 is the nucleotide sequence of BdsA, a bacterial DHN synthase A gene codon-optimized for expression in E. coli (SEQ ID NO: 14). FIG. 17 is the nucleotide sequence of BdsB, a bacterial DHN synthase B gene codon-optimized for expression in E. coli (SEQ ID NO: 15). FIG. 18 is the nucleotide sequence of RppA, a polyketide synthase gene codon-optimized for expression in E. coli (SEQ ID NO: 16).

In one embodiment, the cells contain 80% or more of one or more of the nucleotide sequence numbers 1-16. In another embodiment, recombinant polynucleotides are provided comprising nucleic acid sequences that contain at least about 90% of one or more of the nucleotide sequence numbers 1-16. It will be appreciated by those of skill in the art that divergence in the genetic code may permit variation of the nucleotide sequence without affecting the amino acid sequence of an encoded polypeptide. Thus, the teachings of this invention may be accomplished without using 100% of any particular nucleotide sequence for the genes expressing DHN pathway proteins. Additionally, specific host cells may exhibit “codon-bias” when using nucleotide codons to specify a given amino acid. Accordingly, when synthesizing a nucleic acid as described below, in certain embodiments, it may be desirable to design the nucleic acid such that its frequency of codon usage approaches the frequency of preferred codon usage of the host cell. It will be appreciated by those of skill in the art that this may improve gene expression in the particular host cell.

As mentioned above, the codons for exemplary “DHN-production-related genes,” after optimization for two host cells, P. Putida and E. coli, are shown, in SEQ ID 1-8 (optimized for P. putida) and SEQ ID 9-16 (optimized for E. coli). In some embodiments, only subsets of these 8 genes need to be heterologously provided in the host organism. In other cases, more than one type of recombinant organism may be present having different subsets of the DHN-production-related genes, or there may be different organisms having differently optimized codons for one or more of the DHN-production-related genes.

In some embodiments, an optimized gene may be too large for successful use in a desired expression cassettes. For example, the PKS18 gene optimized for E. coli, PKS18E, and the version optimized for P. putida, PKS18P, may be too large for successful use in expression cassettes suitable for the host cells, so in at least some cases optimized genes may need to be split into two or more fragments. In one example, PKS18E was synthesized with one fragment of 3322 bp and another fragment of 3152 bp, giving two partial sequences. The same was done for PKS18P with partial sequences of 3365 bp and 3109 bp.

In one embodiment, the cells comprise SEQ ID NO.: 1 in whole or in part, along with SEQ ID NOs 2-4. In one embodiment, the cells comprise SEQ ID NO. 1, in whole or in part, along with SEQ ID NOs 6 and 7. In one embodiment, the cells comprise SEQ ID NO. 5 along with SEQ ID NOs 2-4 In one embodiment, the cells comprise SEQ ID NO. 5, along with SEQ ID NOs 6 and 7. In one embodiment, the cells comprise SEQ ID NO. 8 along with SEQ ID NOs 2-4 In one embodiment, the cells comprise SEQ ID NO. 8, along with SEQ ID NOs 6 and 7. In one embodiment, the cells comprise SEQ ID NO.: 9 in whole or in part, along with SEQ ID NOs 10-12. In one embodiment, the cells comprise SEQ ID NO. 9, in whole or in part, along with SEQ ID NOs 14 and 15. In one embodiment, the cells comprise SEQ ID NO. 13 along with SEQ ID NOs 10-12 In one embodiment, the cells comprise SEQ ID NO. 13, along with SEQ ID NOs 14 and 15. In one embodiment, the cells comprise SEQ ID NO. 16 along with SEQ ID NOs 10-12 In one embodiment, the cells comprise SEQ ID NO. 16, along with SEQ ID NOs 14 and 15. In other embodiments, other combinations of the SEQ ID NOs 1-18 may be used.

In one embodiment, individual genes were codon-optimized for each host cell species using a software from ThermoFisher. THERMO FISHER is a registered trademark of Thermo Fisher Scientific, Inc. Thus, 8 genes were codon-optimized for two different host species, so that finally 16 genes were designed. When the genes have been codon-optimized, they may be synthesized for further subcloning to make multiple gene combinations for a complete DHN pathway, as will be further described below. In one embodiment, the single and/or multiple codon-optimized gene(s) may be synthesized by 3 rd party vendor Twist Bioscience. TWIST BIOSCIENCE is a registered trademark of Twist Bioscience Corporation. In order to increase the stability of synthesized gene, the target genes were artificially synthesized and cloned in plasmid backbone vectors, instead of synthesized as linear fragments. The size of pks18 gene was longer than the ability of the vendor's maximum synthesis length. Therefore, 6468 bp of pks18 gene for E. coli and P. putida optimized genes were split by two fragments. In one embodiment, the SBBE cassette for E. coli that is made with Scd1-Brn1-Brn2 for the E. was split into linearized fragments SB (Scd1E and Brn1E) and B (Brn2E), instead of vector cloning. Then these fragments were synthesized.

### Vector Design and Cloning

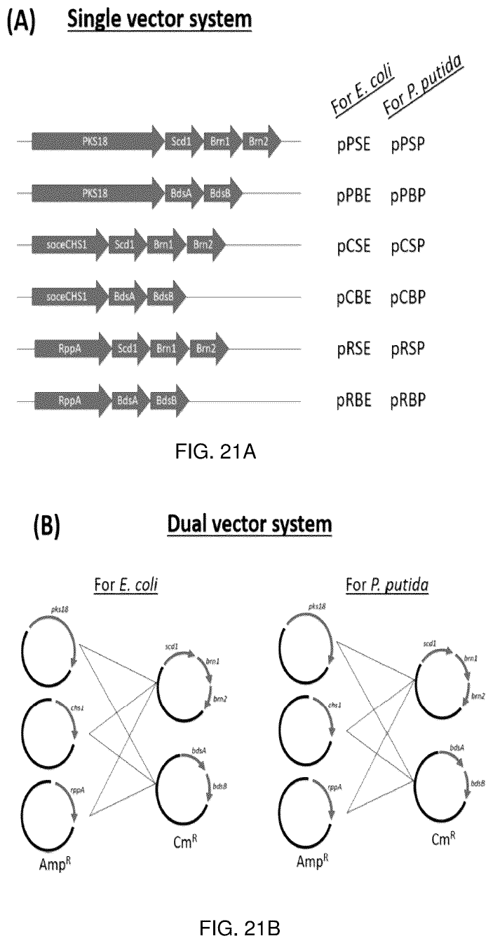

Turning now to FIGS. 21A and B, a single vector and dual vector system are shown. In one embodiment, the PKS18, Scd1, Brn1, and Brn2 genes were codon-optimized for E. coli and were sequentially cloned in a single expression vector designated as “pPSE” in FIG. 21A. These genes were also codon-optimized for P. putida and sequentially cloned in a single expression vector designated as “pPSP.” In another embodiment, the polyketide synthases enzyme gene PKS18 and the genes BdsA and BdsB were codon-optimized for E. coli and were sequentially cloned in a single expression vector designated as “pPBE.” These genes were also codon-optimized for P. putida and sequentially cloned in a single expression vector designated as “pPBP.” In one embodiment, the soceCHS1, Scd1, Brn1, and Brn2 genes were codon-optimized for E. coli and were sequentially cloned in a single expression vector designated as “pCSE” in FIG. 21A. These genes were also codon-optimized for P. putida and sequentially cloned in a single expression vector designated as “pCSP.” In another embodiment, the polyketide synthases enzyme gene soceCHS1 and the genes BdsA and BdsB were codon-optimized for E. coli and were sequentially cloned in a single expression vector designated as “pCBE.” These genes were also codon-optimized for P. putida and sequentially cloned in a single expression vector designated as “pCBP.” In yet another embodiment, the RppA, Scd1, Brn1, and Brn2 genes were codon-optimized for E. coli and were sequentially cloned in a single expression vector designated as “pRSE” in FIG. 21A. These genes were also codon-optimized for P. putida and sequentially cloned in a single expression vector designated as “pRSP.” In another embodiment, the polyketide synthases enzyme gene RppA and the genes BdsA and BdsB were codon-optimized for E. coli and were sequentially cloned in a single expression vector designated as “pRBE.” These genes were also codon-optimized for P. putida and sequentially cloned in a single expression vector designated as “pRBP.”

The pET19b expression vector was selected for the E. coli host cell system. Traditional gene cloning method may be utilized for multiple cloning, including without limitation, ligation, transformation, and the like. The Gibson assembly method may also be applied depending on the desired cloning strategies. The clones may be analyzed by Sanger sequencing analysis to determine the efficacy of the cloning.

In an alternative embodiment, a dual vector expression system may be used as shown in FIG. 21b. Polyketide synthase coding genes may be cloned individually in a vector such as the pET19b vector, for example. Then, other sets of gene sets that encode proteins that facilitate converting polyketide synthases to DHN may be cloned in a pACYCDuet-1 vector. These vectors can be co-transformed into wild-type host strains such as BL21(DE3) wild-type strain in order to test expression parameters. In one embodiment, the polyketide synthase genes PKS18, soceCHS1, and RppA, each codon-optimized for E. coli and P. putida, were individually cloned in a pET19b vector. Each gene in the set of genes Scd1, Brn1, and Brn2 and the set of genes BdsA and BdsB, was codon-optimized for E. coli and P. putida. Each respective gene set was cloned in the pACYCDuet-1 vector. Then each of the three cloned polyketide synthase clone was combined with each of the cloned gene sets. These vectors were co-transformed into wild-type host strains such as BL21(DE3) wild-type strain in order to test expression parameters. These single and dual vector systems may be used in transform desired genes into desired host systems.

In one embodiment, a pETDuet-1 expression vector served as a backbone plasmid for the 6 cassettes made for the codon-optimized genes for E. coli strain. The pETDuet-1 vector has two MCS (multi-cloning sites) regions with two T7 promoters, which is compatible with T7 RNA polymerase expressed by BL21(DE3), a common E. coli host cell used for protein expression. (T7 RNA polymerase is from the T7 bacteriophage and catalyzes the formation of RNA from DNA in the 5′−3′ direction.) Thus, the BL21(DE3) cell strain may be used as a host cell for these vector constructs. However, alternative vector systems and promoters can be considered, for example, non-inducible promoters and/or non-IPTG (IPTG (Isopropyl-(3-D-thiogalactopyranoside) inducible promoters, depending on titer and cell culture status.

In one embodiment, Pseudomonas putida strain does not express T7 RNA polymerase as the BL21(DE3) host cell of E. coli does. Thus, alternative expression systems may be required for the 1,8-DHN expression in P. putida, such as temperature inducible system, xylS/Pm-regulated gene expression system, and/or other chemical inducible systems. For example, xylS/Pm-regulated gene expression vector, pJB861 vector, may be applied for sub-cloning. Pm promoter in this vector is inducible by 1 mM of m-toluate, for example. Examples of alternative expression systems may include but are not limited to:
pJN105 vector, lac promoter
pMMB190 vector, tac promoter
pLLX13 vector, lac promoter
pMEKm12 vector, tac promoter
pJB861 vector, Pm promoter, etc.

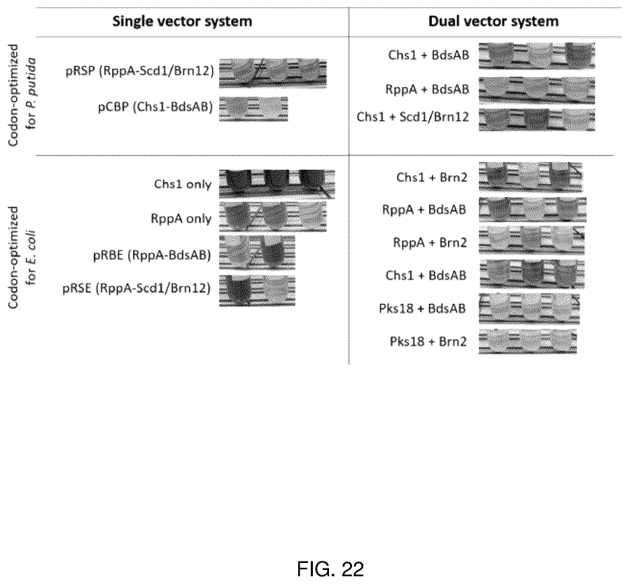

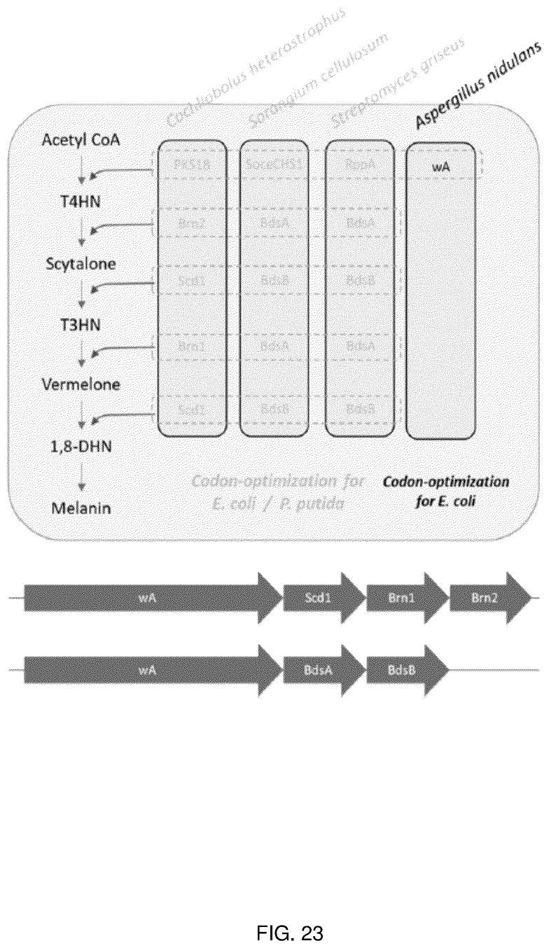

Turning now to FIG. 22, culture test results for the described single and dual vector systems are shown. The tests were performed using BL21(DE3) wild-type strain under common culture conditions, including a Luria-Bertani (LB) liquid media, without any Isopropyl p-D-1-thiogalactopyranoside (IPTG) to induce gene expression. A shake incubator was used at about 37° C. for about 24 hours cultivation time. Single vector system results for the single vectors designated as pRSP, pCBP, pRBE, and pRSE as well as cloned vectors for polyketide synthase genes codon-optimized for E. coli. Results for the following dual vector systems are shown:
Polyketide synthase gene Chs1 cloned for P. putida combined with gene set BdsA and BdsB, codon-optimized for P. putida (designated as Chs1+BdsAB)
Polyketide synthase gene RppA cloned for P. putida combined with gene set BdsA and BdsB, codon-optimized for P. putida (designated as RppA+BdsAB)
Polyketide synthase gene Chs1 cloned for P. putida combined with gene set Scd1, Brn1 and Brn2, codon-optimized for P. putida (designated as Chs1+Scd1/Brn12)
Polyketide synthase gene RppA cloned for E. coli combined with gene set BdsA and BdsB, codon-optimized for E. coli (designated as RppA+BdsAB)
Polyketide synthase gene Chs1 cloned for E. coli combined with gene set BdsA and BdsB, codon-optimized for E. coli (designated as Chs1+BdsAB)
Polyketide synthase gene PKS18 cloned for E. coli combined with gene set BdsA and BdsB, codon-optimized for E. coli (designated as Pks18+BdsAB)
Polyketide synthase gene PKS18 cloned for E. coli combined with Gene Brn2 (designated as Pks18+Brn2)
Polyketide synthase gene RppA cloned for E. coli combined with Gene Brn2 (designated as RppA+Brn2)

As can be seen, some of the cultured samples showed brown colors, suggesting that DHN was reached and subsequently turned to melanin, even though no IPTG inducer was used. The color from these vector systems may indicate the existence of some of the metabolites from the heterologously implanted DHN pathway from the host cell.

In some embodiments, the vector may be any vector, that when introduced into a fungal or bacterial host cell is integrated into the host cell genome and is replicated. It will be appreciated that examples of suitable vectors are publicly available, including the Fungal Genetics Stock Center Catalogue of Strains (found on the internet at www.fgsc.net); Molecular Cloning: A Laboratory Manual, 2.sup.nd ed., Sambrook et al., Cold Spring Harbor, 1989; Current Protocols in Molecular Biology (F. M. Ausubel et al. (eds) 1987, Supplement 30, section 7.7.18).

In certain embodiments, synthesized genes may be combined with promoters, inducers, inhibitors, and other systems in order to better express the genes needed for the production of DHN. In one embodiment, as will be discussed in greater detail below, the use of inhibitory compounds to reduce polymerization of DHN or related compounds may be used. These support systems may be optimized for use in a host as part of the gene, or may be cloned into, or otherwise added to a vector, or utilized in other ways known in the art. They may also be part of an expression cassette. In one embodiment, selected genes were re-organized and re-grouped in order to make multiple cassettes that feasibly expresses 1,8-dihydroxynaphthalene. Finally, six different cassettes were designed.

In certain embodiments, any promoter that functions in the host cell can be used for expression of a polyketide synthase nucleic acid and/or other DHN pathway nucleic acids in the host cell. Initiation control regions or promoters, which are useful to drive expression of such nucleic acids are numerous and familiar to those skilled in the art.

In various embodiments, a polyketide synthase and/or DHN pathway nucleic acid is contained in a low copy plasmid (e.g., a plasmid that is maintained at about 1 to about 4 copies per cell), medium copy plasmid (e.g., a plasmid that is maintained at about 10 to about 15 copies per cell), or high copy plasmid (e.g., a plasmid that is maintained at about 50 or more copies per cell). In some embodiments, a heterologous polyketide synthase and/or DHN pathway nucleic acid is operably linked to a T7 promoter. In some embodiments, a heterologous polyketide synthase and/or DHN pathway nucleic acid is operably linked to a Trc promoter. In some embodiments, a heterologous polyketide synthase and/or DHN pathway nucleic acid is operably linked to a to a Lac promoter. These and other promoters may be used and may, in certain embodiments, by contained in low, medium, or high copy plasmids. In some embodiments, a heterologous polyketide synthase and/or DHN pathway nucleic acid is operably linked to a suitable promoter that shows transcriptional activity in a fungal or bacterial host cell.

In certain embodiments, an expression cassette may include appropriate inhibitors (e.g., laccase inhibitors) to allow DHN to be expressed without significant conversion to melanin or other undesirable reaction products of DHN (e.g., polymers of DHN) when the DHN is expressed.

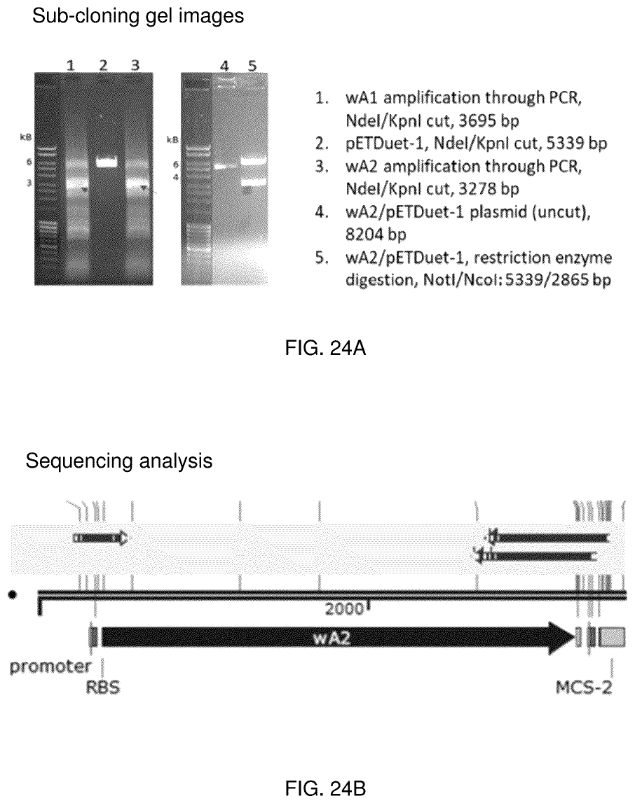

Turning now to FIGS. 24A and 24B, sub-cloning gel images and sequencing analysis are shown respectively for vector constructs for a different polyketide synthase polypeptide, wA, from Aspergillus nidulans was additionally selected for the series of the 1,8-DHN vector constructs. The wA coding gene, wA, was synthesized from a 3rd party and cloned in Scd1-Brn1-Brn2 cassette and BdsA-BdsB cassette in the overexpression vectors with results shown in FIG. 24A. The successful vector sample was confirmed through restriction enzyme digestion pattern and sanger sequencing analysis, as shown in FIG. 24B. Gel images for five samples shown in FIG. 24A are shown. Sample #4 and #5 showed the DNA band of the wA in pET19b vector of the circular plasmid vector and the restriction enzyme digested vector.

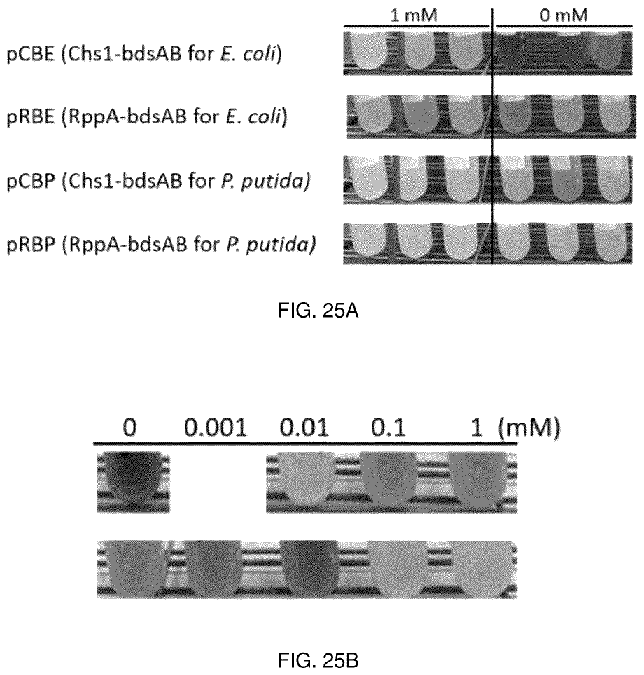

Turning now to FIGS. 25A and 25B vector test results are shown. To find out the best efficiency of IPTG concentration to the single vector system cultivation, several concentrations of IPTG were tried to test the culture for four different vector sets, such as pCBE (Chs1-BdsA-BdsB for E. coli), pRBE (RppA-BdsA-BdsB for E. coli), pCBP (Chs1-BdsA-BdsB for P. putida), and pRBP (RppA-BdsA-BdsB for P. putida). From pRBE vector results the color difference between 1 mM of IPTG induction and no IPTG induced samples was less distinguishable. However, the results of different concentration samples showed 0.1 mM of IPTG concentration was the best color expression, suggesting that this IPTG concentration may yield the best DHN expression.

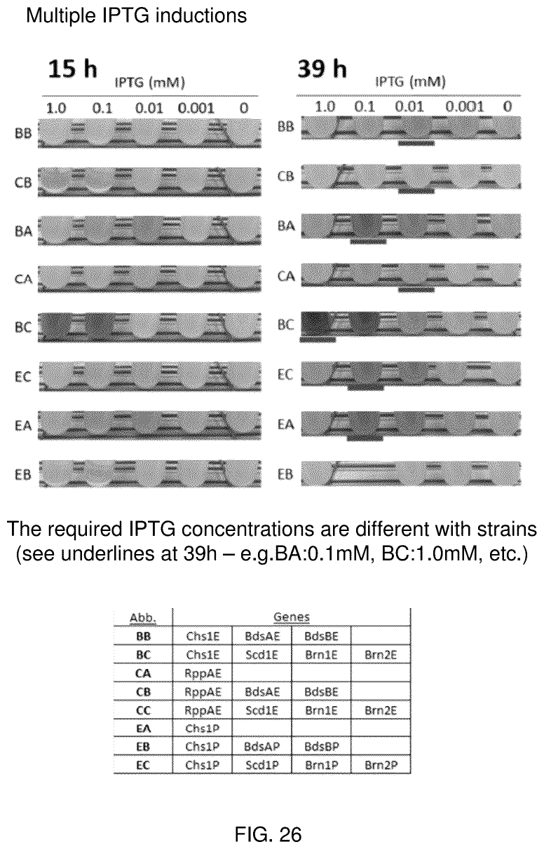

Turning now to FIG. 26, the results of another trial to find the best concentration of IPTG for vector constructs was conducted for 15 h and 39 h post-induction samples at 37° C. culture. IPTG concentrations were adjusted at 1.0, 0.1, 0.01, 0.001, and 0 mM for all group of vector constructs. M9 minimal media containing 10 g/L glucose was used as a culture media. From the results of 39-hour post-induction samples, specific IPTC concentrations were possibly selected by the color intensity of the samples. A shortened form of nomenclature for each tested vector is used because of the complexity of long vector names. 0.01 mM of IPTG was the best for the BB, CB, and CA vectors, 0.1 mM was for the BA, EC, EA vectors, and 1.0 mM IPTG was the best for the BC vector in E. coli cells. This induction parameter was fixed for further experiments.

### Exemplary Transformation Methods

Polyketide synthase and/or DHN pathway nucleic acids or vectors containing them, together with any support materials such as promoters, inducers, inhibitors, stabilizers, and the like, can be inserted into one or more of the host cells described herein using standard techniques for expression of the encoded polyketide synthase and/or DHN pathway polypeptides. Introduction of a DNA construct or vector into a host cell can be performed using techniques such as transformation, electroporation, nuclear microinjection, transduction, transfection (e.g., lipofection mediated or DEAE-Dextrin mediated transfection or transfection using a recombinant phage virus), incubation with calcium phosphate DNA precipitate, high velocity bombardment with DNA-coated microprojectiles, and protoplast fusion. General transformation techniques are known in the art (see, e.g., Current Protocols in Molecular Biology (F. M. Ausubel et al. (eds) Chapter 9, 1987).

### Exemplary Cell Culture Media

In one embodiment, cells are in a culture having conditions for producing DHN. Such conditions include a carbon source capable of being metabolized by the host cell. In some embodiments, the carbon source is a carbohydrate and may include, without limitation, a monosaccharide, a disaccharide, an oligosaccharide, a polysaccharide, and invert sugar, a syrup, a glycerol, a glycerin, a dihydroxyacetone, a one-carbon source, a plant or vegetable oil, an animal fat, an animal oil, a fatty acid, a lipid, a phospholipid, a glycerolipid, a monoglyceride, a diglyceride, a triglyceride, a microbial or plant protein or peptide, a renewable carbon source, a biomass carbon source, a yeast extract, a polymer, an acid, an alcohol, an aldehyde, a ketone, an amino acid, a succinate, a lactate, an acetate, an ethanol, portions of the foregoing, combinations of the foregoing, and the like. In some embodiments, the carbon source is a product of photosynthesis, including, but not limited to, glucose. In one embodiment, the carbon source is hydrolyzed biomass.

In some embodiments, the concentration of the carbohydrate is at least about 5 grams per liter of medium wherein the volume of medium include both the volume of the cell medium and the volume of the cells. In other embodiments, the concentration of the carbohydrate is at least about 10, 15, 20, 30, 40, 50, 60, 80, 100, 150, 200, 300, 400, or more g/L. In some embodiments, the concentration of the carbohydrate is between about 50 and about 400 g/L, such as between about 100 and about 360 g/L, between about 120 and about 360 g/L, or between about 200 and about 300 g/L. In some embodiments, this concentration of carbohydrate includes the total amount of carbohydrate that is added before and/or during the culturing of the host cells.

In some embodiments, the cells are cultured under limited glucose conditions, wherein the amount of glucose that is added is less than or about 100% of the amount of glucose that is consumed by the cells. In other embodiments, the amount of glucose that is added to the culture medium is approximately the same as the amount of glucose that is consumed by the cells during a specific period of time. In some embodiments, the rate of cell growth is controlled by limiting the amount of added glucose such that the cells grow at the rate that can be supported by the amount of glucose in the cell medium. In some embodiments, glucose does not accumulate during the time the cells are cultured. In various embodiments, the cells are cultured under limited glucose conditions for greater than or about 1, 2, 3, 5, 10, 15, 20, 25, 30, 35, 40, 50, 60, or 70 hours. In various embodiments, the cells are cultured under limited glucose conditions for greater than or about 5, 10, 15, 20, 25, 30, 35, 40, 50, 60, 70, 80, 90, 95, or 100% of the total length of time the cells are cultured. While not intending to be bound by any particular theory, it is believed that limited glucose conditions may allow more favorable regulation of the cells.

In some embodiments, the cells are cultured in the presence of an excess of glucose. In particular embodiments, the amount of glucose that is added is greater than about 105%. In some embodiments, the amount of glucose that is added is greater than about 110, 120, 150, 175, 200, 250, 300, 400, or 500% or more of the amount of glucose that is consumed by the cells during a specific period of time. In some embodiments, glucose accumulates during the time the cells are cultured.

In one embodiment, a biomass carbon source is a lignocellulosic, hemicellulosic, or cellulosic material including without limitation, grass, wheat, wheat straw, bagasse, sugar cane bagasse, soft wood pulp, corn, corn cob or husk, corn kernel, fiber from corn kernels, corn stover, switch grass, rice hull product, or a by-product from wet or dry milling of grains such as corn, sorghum, rye, triticate, barley, wheat, distillers grains, combination of the foregoing, and the like. Exemplary cellulosic materials include wood, paper and pulp waste, herbaceous plants, and fruit pulp. In some embodiments, the carbon source includes any plant part, such as stems, grains, roots, or tubers. In some embodiments, all or part of certain plants may be used as a carbon source, including without limitation, corn, wheat, rye, sorghum, triticate, rice, millet, barley, cassava, legumes, such as beans and peas, potatoes, sweet potatoes, bananas, sugarcane, and/or tapioca. In some embodiments, the carbon source is a biomass hydrolysate, such as a biomass hydrolysate that includes both xylose and glucose or that includes both sucrose and glucose. In some embodiments, the carbon source may be treated before being added to the cell culture medium.

In some embodiments, the concentration of the carbon source is equivalent to at least about 0.1, 0.5, 1, 1.5 2, 3, 4, 5, 10, 15, 20, 30, 40, or 50% glucose (w/v). The equivalent amount of glucose can be determined by using standard high performance liquid chromatography (HPLC) methods with glucose as a reference to measure the amount of glucose generated from the carbon source. In some embodiments, the concentration of the carbon source is equivalent to between about 0.1 and about 20% glucose, such as between about 0.1 and about 10% glucose, between about 0.5 and about 10% glucose, between about 1 and about 10% glucose, between about 1 and about 5% glucose, or between about 1 and about 2% glucose.

### Exemplary Cell Culture Conditions

Materials and methods suitable for the maintenance and growth of bacterial cultures are well known in the art. Exemplary techniques may be found in Manual of Methods for General Bacteriology (Gerhardt et al., eds), American Society for Microbiology, Washington, D.C. (1994) or Brock in Biotechnology: A Textbook of Industrial Microbiology, Second Edition (1989) Sinauer Associates, Inc., Sunderland, Mass. In one embodiment, cell culture conditions are conducive to fermentation within the cell.

The cells may be grown and maintained at appropriate temperatures, pH balances, oxygenation levels, and/or other process parameters to generate a desired amount of DHN. In some embodiments, the cells are grown and/or maintained at a temperature of at least about 20° C. In some embodiments, the cells are grown and/or maintained below about 37° C. In some embodiments, the cells are grown and/or maintained at temperatures between about 28° C. and about 35° C. inclusive. In one embodiment, the cultures are maintained at low temperature to reduce polymerization or melanin formation. In one embodiment, the culture temperatures may be less than 0° C. In one embodiment, the culture and/or process temperature may be from about −80° C. to about −10° C. In some embodiments, the cells are grown and/or maintained in a medium having between about 6% to about 84% CO₂. In certain embodiments, the pH of the cell medium is between about 5 and about 9 inclusive. In some embodiments, cells are grown at 35.degree. C. in an appropriate cell medium. In some embodiments cell mediums or cultures may be kept at particular process parameter levels for certain amounts of time. In some embodiments, the culturing of cells for biosynthesis of DHN may be conducted under low-oxygen conditions such as under a nitrogen atmosphere or an atmosphere with less than 20%, 15%, 10%, 5%, or 1% oxygen. Storage of DHN or related compounds may also be in a low-oxygen atmosphere such as in nitrogen or carbon dioxide. In certain embodiments, the methods of the present invention may be carried out at least in part under a low oxygen atmosphere having less than 10% oxygen. In other embodiments, the methods of the present invention may be carried out at least in part under a low oxygen atmosphere having less than 1% oxygen.

Cell cultures or mediums may have aerobic, anoxic, or anaerobic conditions based on the requirements of the host cells. The cells may be grown using batch, fed-batch, and/or continuous processes. In some embodiments, bottles of liquid culture are placed in shakers in order to introduce oxygen to the liquid and maintain the uniformity of the culture. In some embodiments, an incubator is used to control the temperature, humidity, shake speed, and/or other conditions in which a culture is grown. If desired, a portion or all of the cell medium can be changed to replenish nutrients and/or avoid the buildup of potentially harmful metabolic byproducts and dead cells. In the case of suspension cultures, cells can be separated from the media by centrifuging or filtering the suspension culture and then resuspending the cells in fresh media. In the case of adherent cultures, the media can be removed directly by aspiration and replaced. In some embodiments, the cell medium allows at least a portion of the cells to divide for at least or about 5, 10, 20, 40, 50, 60, 65, or more cell divisions in a continuous culture.

### Exemplary Inhibitor Compounds

DHN can be converted to melanin (melanogenesis) by p-diphenol oxidase and/or related enzymes along the DHN melanin pathway. Therefore, removal and/or inhibition of such enzymes or reagents that may drive undesired reactions with DHN may enhance production of DHN. This may be done by gene knock-out on DNA level such as a homologous recombination gene knock-out method, gene knock-down on RNA level such as RNA interference (RNAi), increasing the concentration of inhibition molecules, etc., and the use of other gene control techniques involving, for example, zinc finger proteins and other means.

In one embodiment inhibition compounds are added at appropriate times or locations in the process to reduce the risk of melanin production or the polymerization of DHN or related species or its precursors. Such inhibition compounds can include compounds that reduce melanin expression in fungi or other organisms such as tricyclazole or other anti-fungal agents, as well as antioxidants able to quench free radicals or otherwise inhibit polymerization reactions, including ascorbic acid (Vitamin C) and salts or esters thereof; retinoids; resveratrol (3′,4′,5′-trihydroxy-trans-stilbene); cinnamic acid and its derivatives such as ferulic acid; glycolic acid; hydroquinone; coumaric acid; kojic acid; azelaic acid and other dicarboxylic acids and the like.

In one embodiment, removal and/or inhibition of such enzymes may reduce or block melanin synthesis and increase the amount of DHN in the host cell and the amount of DHN that can be obtained from a culture. In one embodiment the p-diphenol oxidase is modified to control of melanin conversion ratios. In another embodiment, gene knock-out strategies are applied in the host cell DNA, including without limitation, using RNA interference (RNAi), increasing the concentration of other inhibition molecules, using various chemical inhibitors, and the like.

Genes encoding p-diphenol oxidase are found in various species in the wild with different names, such as cutO from Rhodobacter capsulatus, abr2 from Aspergillus fumigatus, and afAbr2 from Talaromyces (Penicillium) marneffei. Such genes occur and are called cueO and copA-II in Escherichia coli and Pseudomonas putida host cells respectively. CopA-II enzyme is one of the multicopper oxidase enzymes in P. putida with 2010 base pairs. Similarly, two enzymes, cueO (1551 bp) and pgeF, are found from E. coli as MCO and/or laccase. Thus, in some embodiments, these two genes may be removed from a host cell through a knock-out method, including without limitation, the KEIO method, to promote increased DHN production and decreased loss by conversion to melanin.

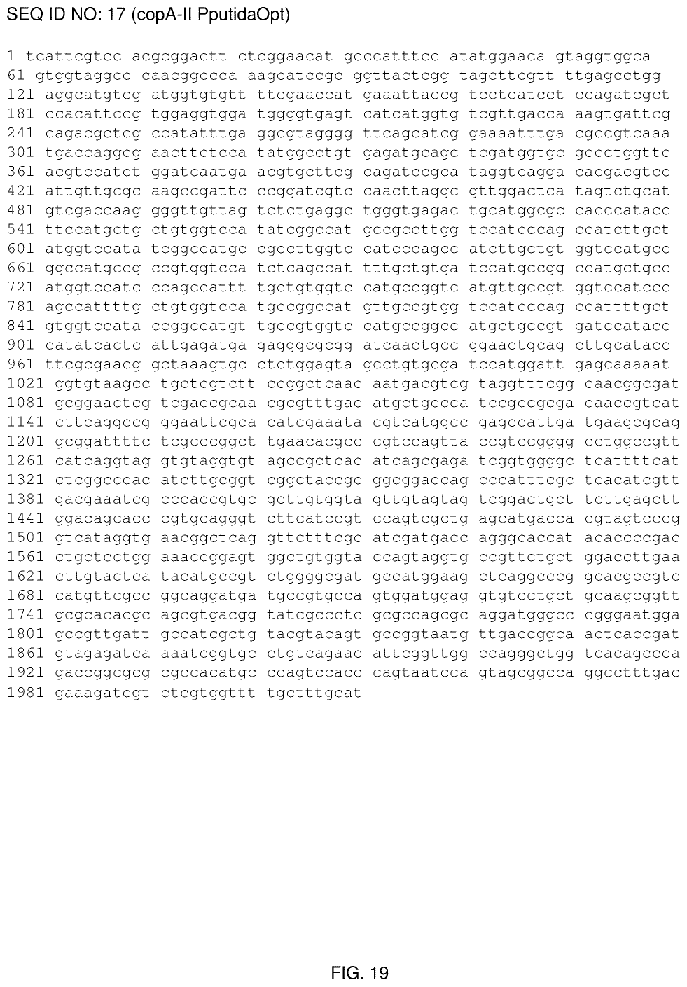

FIG. 19, the nucleotide sequence of the p-diphenol oxidase gene copA-11, codon-optimized for expression in P. putida, identified as SEQ ID NO: 17.

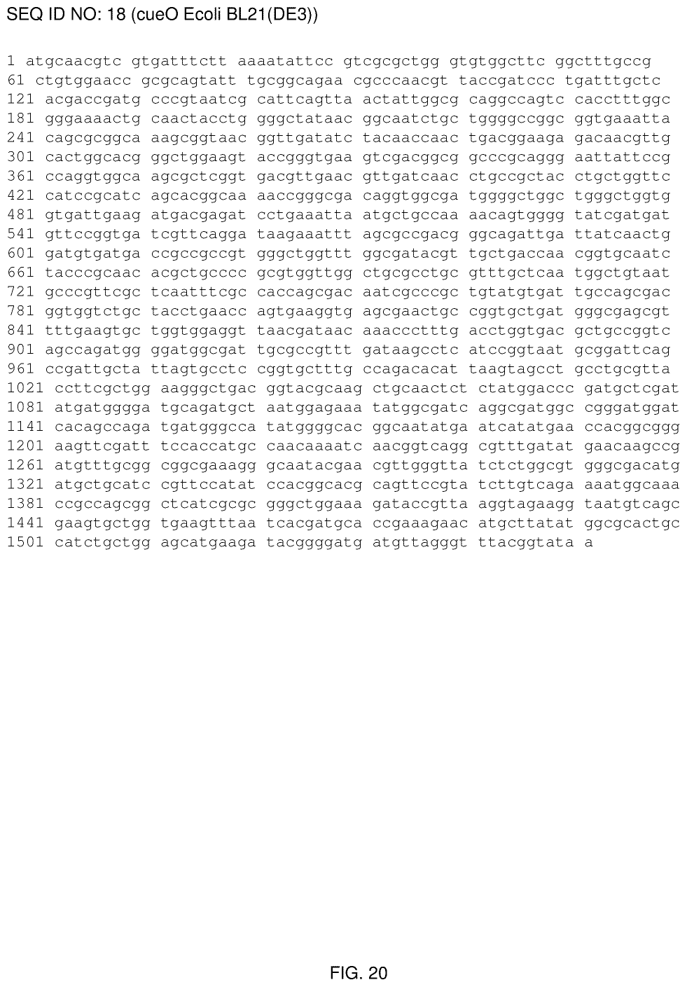

FIG. 20 is the nucleotide sequence of the p-diphenol oxidase gene cueO, codon-optimized for expression in E. coli, identified as SEQ ID NO: 18.

In some embodiments, compounds are used for inhibiting melanin formation (melanogenesis). These “melanogenesis inhibition compounds” may serve to block the polymerization of DHN or related compounds or its precursors, and may include anti-fungal agents that reduce expression of melanin in fungi or other compounds expressed by various microorganisms. Melanogenesis inhibition compounds can be any compounds that reduce melanin formation in any context. In one embodiment, an anti-fungal agent may be used to inhibit melanogenesis, including without limitation, tricyclazole, chlobenthiazone, carpropamid [((1RS,3SR)-2,2-dichloro-N—[(R)-1-(4-chlorophenyl)ethyl]-1-ethyl-3-methylcyclopropanecarboxamide)], diclocymet, fenoxanil, 6-hydroxyflavanone, 4-hydroxy-7-methoxy-3-phenyl-coumarin, 7-hydroxy-4-phenyl-coumarin and 7-hydroxy-3,4,8-trimethylcoumarin, 2,3,4,5,6-pentachlorobenzyl alcohol, cerulenin, 3,4-dihydroxyphenylalanine, pyroquilon, phthalide, and the like. Tricyclazole, for example, may be obtained from Eli Lilly Research Laboratories (Greenfield, Ind.), pyroquilon from Ciba-Geigy Ltd. (Basel, Switzerland), and phthalide from Kureha Chemical Ind. Co. Ltd. (Tokyo, Japan). Tricyclazole and pyroquilon may be dissolved in ethanol and may be present in a culture medium. They may be present alone or together or may be combined, alone or together, with any other inhibitor, such that the inhibitor concentration of any one, two, or more inhibitors may range from 5 ppm to 20,000 ppm or from 5 ppm to 5000 ppm or from 5 ppm to 500 ppm on a mass basis in the culture medium, and may be added initially or at a later stage in the process.

Laccases are enzymes believed to play a role in converting DHN to melanin in some systems. Accordingly, in some embodiments, laccase inhibitors may be used, including without limitation, sodium azide, dithiothreitol, thioglycolic acid, cysteine, diethyl-dithiocarbamic acid, other sulfhydryl organic compounds, zineb, other dithiocarbamate compounds, derivatives or salts thereof, and the like. Halides such as fluoride, bromide, or chloride ions may also be used to inhibit laccase. Cyanide such as potassium cyanide may also be used, however it will be appreciated that a subsequent conversion step to remove or detoxify the cyanide may be desirable.

Some antioxidants that may quench free radicals or otherwise inhibit polymerization reactions may be used at melanogenesis inhibitors, including without limitation, ascorbic acid (Vitamin C) and salts or esters thereof; retinoids, retinol, retinol combined with hydroxyquinone, tretinoin, and tretinoin combined with hydroxyquinone (e.g., 0.01% to 0.5% tretinoin or other retinoid plus 0.01% to 1% hydroxyquinone); niacinamide/nicotinamide and derivatives, salts, or esters thereof; resveratrol (3′,4′,5′-trihydroxy-trans-stilbene); cinnamic acid and its derivatives such as ferulic acid, stabilized ascorbic acid compositions, glycolic acid, hydroquinone, coumaric acid such as p-coumaric acid; kojic acid; uric acid; bilirubin; albumin; various thiols such as trypanothione, mycothiol and glutathione; ubiquinol or coenzyme Qio, azelaic acid and other dicarboxylic acids such as adipic acid, succinic acid, glutaric acid, pimelic acid, etc.; tolprocarb, a carbamate ester that is the 2,2,2-trifluoroethyl ester of [(25)-3-methyl-1-(4-methylbenzamido)butan-2-yl]carbamic acid, diphenylamines such as those disclosed in U.S. Pat. Nos. 2,180,936, 3,655,559, 3,944,492, 5,750,787, 6,315,925, and 2,530,769; the diarylamines disclosed in U.S. patent Ser. No. 10/323,017, and the like.

In one embodiment, Salidroside, an active component of Rhodiola rosea, may be used as melanin compound inhibitor, as well as other components that have free radical scavenging effects. In one embodiment, Paeonol isolated from Moutan Cortex Radicis may also be used as well as extracts of Phellinus linteus, a fungus that grows in the wild on mulberry trees and is often used in anti-cancer therapy, sometimes with extracts of Reishi and Maitake. Arbutin, a glycosylated hydroquinone extracted from the bearberry plant in the genus Arctostaphylos and in other plants of the family Ericaceae, may also be used as a melanogenesis inhibitor. Flavonoids or polyphenols may be used, including the flavanol quercetin, rutin, the polyphenol curcumin, and flavones, flavonols, flavanols, flavanones, isoflavones, proanthocyanidins, and anthocyanins, including catechins, hesperetin, cyaniding, daidzein (soy), proanthocyanidins (apple, grape, cocoa) and the like.

In one embodiment, phenolic acid such as caffeic acid, chicoric acid, salicyclic acid, or chlorogenic acid, and lignans such as polyphenols derived from phenylalanine may be used and a melanogenesis inhibitor. Other melanin inhibitor compounds may include glutathione, its metabolite pyroglutamic acid, vitamin E, ranexamic acid, piperine, ginger extract, lipoic acid, 2-mercaptobenzothiazole, cryptoxanthins, beta-carotene, mung bean extracts and related products such as the natural ferment Pracparatum mungo (Lu-Do Huang). Other plant extracts with melanin inhibitor characteristics may be used, including without limitation, Nicotiana glauca Gram (Tabaquillo), Nicotiana trigonophylla Dunal (Tabaquillo delgado), Solanum rostratum Dunal (Abrojo), Solanum nigrescens Mart. & Gal. (Hierba Mora), and the like. Extracts or the purified alkaloids from these plants may also be used in certain embodiments.

Tropolone and derivatives thereof may also be used. Tropolone compounds may include dolabrins, dolabrinols, thujaplicins, thujaplicinols, stipitatic acid, stipitatonic acid, nootkatin, nootkatinol, puberulic acid, puberulonic acid, sepedonin, 4-acetyltropolone, pygmaein, isopygmaein, procein, chanootin, benzotropolones (e.g., purpurogallin, crocipodin, goupiolone A and B), theaflavin and its derivatives, bromotropolones, tropoisoquinolines and tropoloisoquinolines (e.g., grandirubrine, imerubrine, isoimerubrine, pareitropone, pareirubrine A and B), colchicine, colchicone, and the like. Other melanogenesis inhibitors may include benomyl, ferimzone, Tiadinil, Orysastrobin, and the like. Tolprocarb derivatives may be used,

Inhibitor compounds may be present in the culture media at levels of 5 ppm to 10,000 ppm, such as from 5 ppm to 1000 ppm, or 5 ppm to 500 ppm, or 10 ppm to 300 ppm.

Depending on the system needs, inhibitor compounds may be hydrophilic or lipophilic. For example, Vitamin C, uric acid, bilirubin, albumin, and thiols are hydrophilic, radical-scavenging antioxidants, while vitamin E and ubiquinol are lipophilic radical-scavenging antioxidants. In certain embodiments one or more of the foregoing melanogenesis inhibition compounds, gene knockout materials, laccasse inhibitors, antioxidants, herbs, plants, extracts or any materials that inhibit DHN from becoming melanin along the DHN melanin pathway (collectively, “melanogenesis inhibition compounds” or “melanin inhibitors”) may be used.

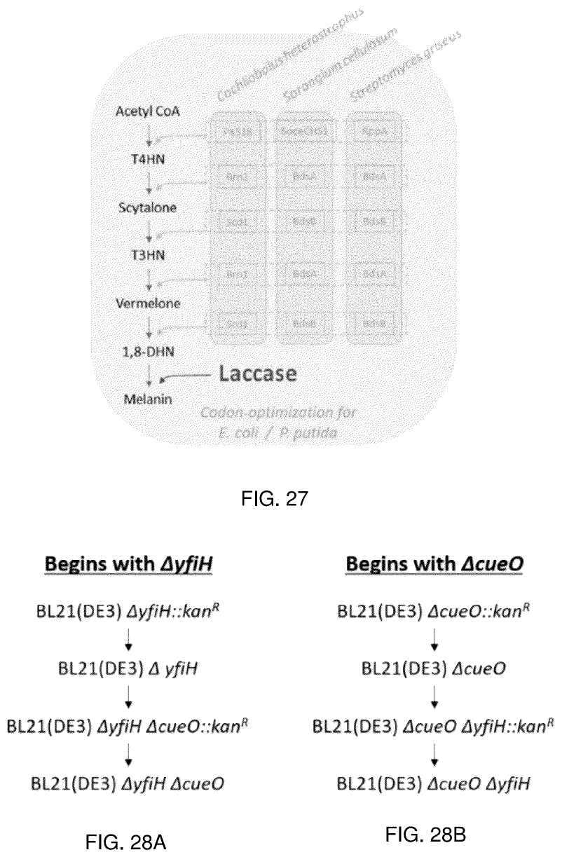

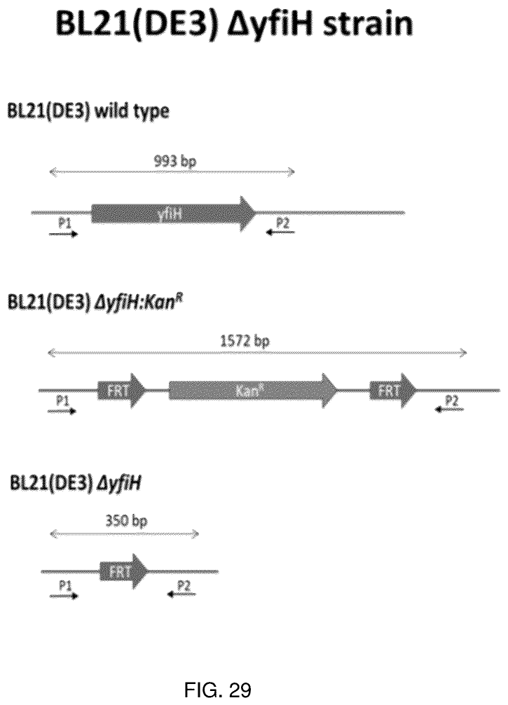

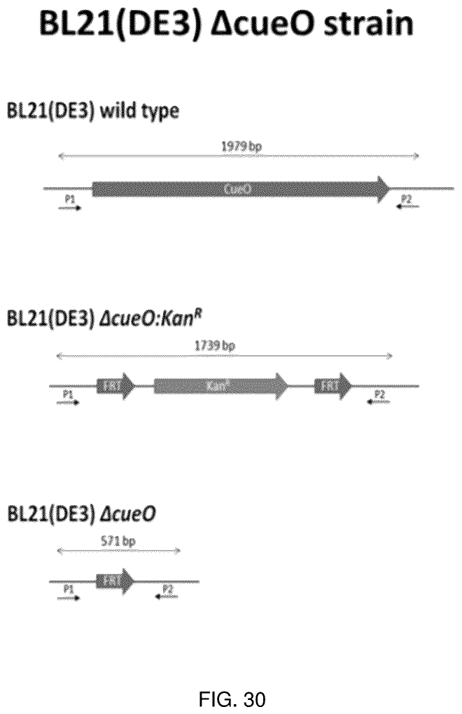

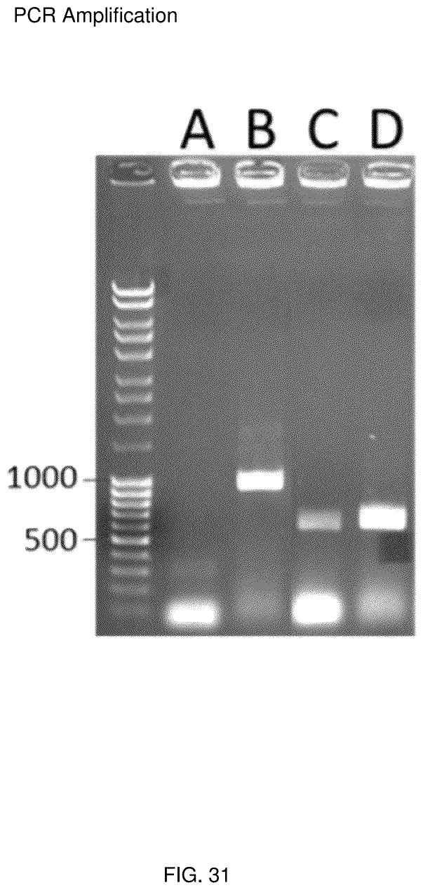

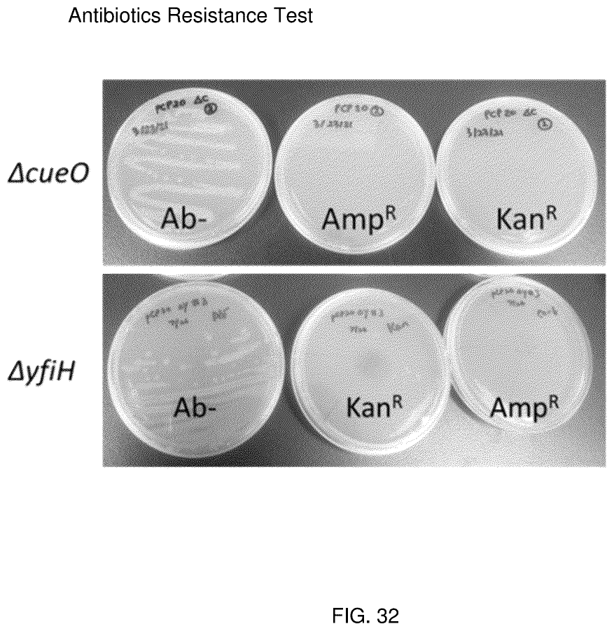

Turning now to FIGS. 27-33, the cells and/or cell media may include inhibitor compounds to facilitate the removal of genes coding laccasse, an enzyme used to convert DHN to melanin. In FIG. 27, the laccasse enzyme is shown as part of an overall DHN melanin pathway. Two laccase coding genes were found from the E. coli enzyme database (ecocys.org); namely the multicopper oxidase, CueO and the polyphenol oxidase, YfiH. Removal of these laccase coding genes may prevent the conversion of DHN to melanin leading to a higher accumulations of DHN in the cell culture media. FIG. 28A shows the gene knockout strategy for removing yfiH and FIG. 28B shows the gene knockout strategy for removing cueO. In one embodiment, these genes were removed or limited through the homologous gene recombination method, known as the KEIO method, using the Kan-FRT cassette vector system. First, about 1 to 2 kilobase of these two genes were replaced with Kan-FRT cassette. Then the Kan-FRT was removed by the FLP recombinase enzyme. The double knock-out strain was completed at the end of the project period. See FIGS. 29 and 30. The knock of these genes were confirmed through PCR amplification (FIG. 31) and antibiotics screening test (FIG. 32).

### Media Tests

Metabolites from the DHN pathway, including T4HN, Scytalone, T3HN, Vermelone, DHN, and melanin are produced in the host cell and released outside of the host cell to exist in the culture media. To test the relationship between DHN among these metabolites and the components of the culture media, DHN were added and cultured to the M9 minimal media and LB media with and without BL21(DE3) cells. The samples were cultured with 10 g/L glucose, 0.1 mM of IPTG, and 0.5 ug of the DHN compound in the media. As a result, the addition of the DHN caused some cloudy particles in LB liquid media without growing cells in the tubes.

To test for the leaked expression of vectors in E. coli cells, designed vectors were transformed in E. coli host cells and spread on LB-agar plates for screening for the successful transformants. The agar plate for this process contained essential nutrients such as yeast extract, tryptone, and sodium chloride. But there were no materials for the induction of the T7 promoter on the pET19b expression vector system. However, some of the LB agar plates that harbor the DHN pathway gene containing vectors expressed color.

An anaerobic culture test was performed to see how oxygen may affect the enzymatic function of the laccase/laccase-like enzymes for the melanin conversion from DHN. In this test, some of the vector transformants were cultured under anaerobic culture conditions. The vector of Chs1-BdsA-BdsB for P. putida codon-optimized clone was transformed in BL21(DE3) ΔyfiH::kan^{R} strain and inoculated in M9 minimal media containing 10 g/L glucose and 0.01 mM IPTG in 37° C. chamber. After two days, the samples stayed without any color expression.

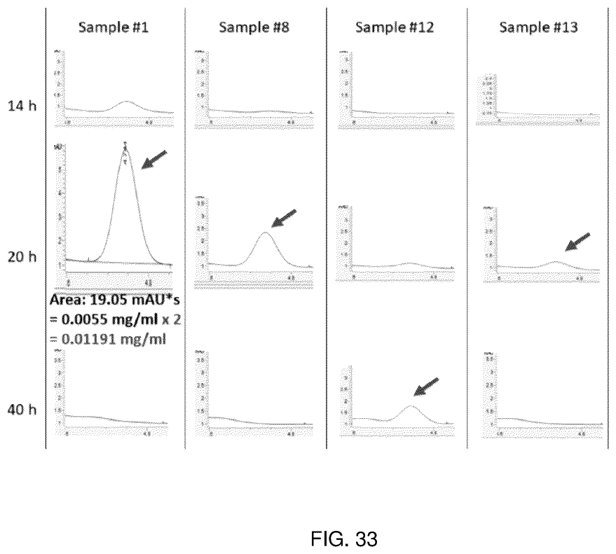

Media tests for HPLC measurement from the vector system in E. coli strain were also conducted. The single vector systems described above were transformed in BL21(DE3) ΔyfiH::kan^{R} strain with LB and M9 containing 0 or 0.1 mM IPTG and 10 g/L glucose as a carbon source. All cultured samples were prepared for the HPLC runs with the standard sample of 1,8-DHN as 0.005 mg/ml concentration. Among the samples, a peak appeared from the 27 hours cultured the Chs1-Scd1-Brn1-Brn2 for P. putida (named as ‘EC’ on the FIG. 12) vector harboring sample. The retention time was around 4.58 min, which is closed to the standard DHN (4.799 min). From the follow-up test, four samples were prepared and measured by the HPLC with 14 h, 20 h, and 40 h post-induced ‘EC’ samples. The four samples were inoculated from the same agar plate; thus, they are considered identical colonies. Turning to FIG. 33, the peaks appeared at different post-induction times. The largest concentration of DHN from these samples was 11.91 mg/L (FIG. 12).

The culture parameters of temperature, media composition, post-induction cultivation time, and IPTG concentration were optimized through trial and error. Here, two different glucose concentrations as a sole carbon source were tested and analyzed through HPLC. The BC vector in BL21(DE3) ΔcueO strain, which is a short name of Chs1-Scd1-Brn1-Brn2 cassette, showed 12.2 mg/L concentration of 1,8-DHN which was the highest titer. However, the other samples from different colonies, even though they came from the same vector in the same cell strain showed less titers.

Based on the culture parameter optimization, the best culture conditions were established. The cultured cell was harvested from 18 h to 25 h post-induction for 16 ml test tubes culture, and 18 h to 70 h post-induction for 250 ml flasks culture. The results for the 16 ml test tubes are shown in Table 1.

TABLE 1
16 ml Tubes: 5 ml Culture Volume

| | 18 | 19 | 20.4 | 21.1 | 23.5 | 24.75 |
|---|---|---|---|---|---|---|
| BCΔc-0 | 0.771763 | ND | 0.61757 | 0.891526 | ND | 0 |
| BCΔc-1 | ND | 0.758289 | 0.487994 | ND | ND | 0 |
| BCΔc-2 | ND | 0.661255 | ND | 0.734927 | ND | ND |
| BCΔy-1 | 0 | 0.680134 | 10.645611 | 0.51746 | ND | 0 |
| BCΔy-2 | 0 | ND | ND | ND | ND | ND |
| ECΔc-1 | 0.614706 | 0.588278 | 0.757661 | 0.864296 | 0.528418 | ND |
| ECΔy-0 | ND | 0.48654 | 0 | ND | 0 | 0 |

The results for the 250 ml test tubes are shown in Table 2.

TABLE 2
250 ml Flasks: 50 ml Culture Volume

| | 18 | 19 | 20.4 | 21.1 | 23.5 | 24.75 | 42 | 48.75 | 68 |
|---|---|---|---|---|---|---|---|---|---|
| BCΔc-1 | 1.075856 | 1.810088 | 4.6885221 | 4.430133 | 5.38615 | 4.087325 | ND | ND | ND |
| BCΔy-1 | 0.619483 | 1.318704 | 2.173398 | 4.021549 | 4.112483 | 2.613278 | ND | ND | ND |
| ECΔc-0 | 0 | 0 | 0 | 0 | 0 | 0 | 0 | 0 | 0 |
| ECΔc-1 | ND | ND | 0.732715 | 1.575611 | 4.569002 | 5.8026 | 0 | ND | ND |
| BCΔc-0 | 0 | 0 | 0 | 0 | 0 | 0 | 0 | 0 | 0 |
| ECΔy-0 | 0 | 0 | 0 | 0 | 0 | 0 | 0 | 0 | 0 |

### EXAMPLES

Expression of 1,8-DHN in two different host species (prophetic example)

1. E. coli System

The BL21(DE3) host cell is used for the DHN expression cassettes in pETDuet-1 vector. The overall expression protocol in one embodiment may be as follows below:
1. Prepare the plasmid vector constructs.
2. Perform transformation of the plasmids into commercial BL21(DE3) competent
cell through the heat-shock method and/or electroporation method. 3. The transformed cell harboring the plasmid is spun down through centrifugation and resuspend in 100 ul of DI water. The resuspended cell is spread on LB agar plate that contains ampicillin (100 mg/L) as a selection marker. The plates are incubated in 37° C. chamber overnight.
4. From the LB agar plate, multiple colonies are inoculated in LB liquid media with ampicillin, then the cell culture is placed in a shake incubator at 37° C. overnight.
5. Fully grown cells are sub-cultured in a proper volume of LB liquid media with ampicillin, and is cultured until OD600 reaches 0.5. Then 1 mM of IPTG (Isopropyl-(3-D-thiogalactopyranoside) is added in the cell culture, followed with treatment of the culture in a shake incubator culture for multiple days. After post IPTG induction, a proper amount of cell cultures are collected from the culture flasks or tubes. These samples are centrifuged to remove the pellet, and the pure SPNT is filtrated by 0.22 um syringe filter. These samples are analyzed by HPLC.
6. In order to obtain the best 1,8-DHN expression titer in flask culture, variable culture conditions can be applied, modified and tested such as temperature, concentration of carbon sources, pH of media, IPTG concentration, media compositions, and the like.
7. Based on the HPLC analysis, the best culture condition can be determined and be applied to scale-up culture using bioreactors.
   2. P. putida System
1. Prepare the plasmid vector constructs.
2. Prepare proper Pseudomonas strains, such as P. putida. Then, prepare the cell as competent cell if it is required.
3. Perform transformation of the plasmid into the Pseudomonas competent cell through the heat-shock method, electroporation method, and/or Yoshida method, but the process is not limited to these methods.
4. Successfully transformed cell is grown on proper nutrition media plates at a proper temperature.
5. Successfully grown colonies from the agar plate are inoculated into proper nutrition liquid culture media with antibiotics, then cultured overnight in a shake incubator.
6. When the inoculated cells are fully grown, a proper amount of cells will be inoculated into liquid media in flasks. When the Pseudomonas cell has reached the proper OD600 value (e.g., 0.5), a specific induction method is applied in order to start the gene expression.
7. The flasks are cultured in a shake incubator for multiple days. After post-induction, a proper amount of cell cultures will be collected from the culture flask. These samples are centrifuged to remove the pellet, and the SPNT is filtrated by 0.22 um syringe filter. This sample is analyzed by HPLC.
8. In order to obtain the best 1,8-DHN expression titer in flask culture, variable culture conditions will be modified and tested such as temperature, concentration of carbon sources, pH of media, induction method, media compositions, and the like.
9. Based on the HPLC analysis, the best culture condition will be determined and be applied to scale-up culture using bioreactors.

The successful vector constructs can be compared with each other in order to select the best or better cassette or to select the most effective host cells, the most effective promoters, inhibitors, and the like.

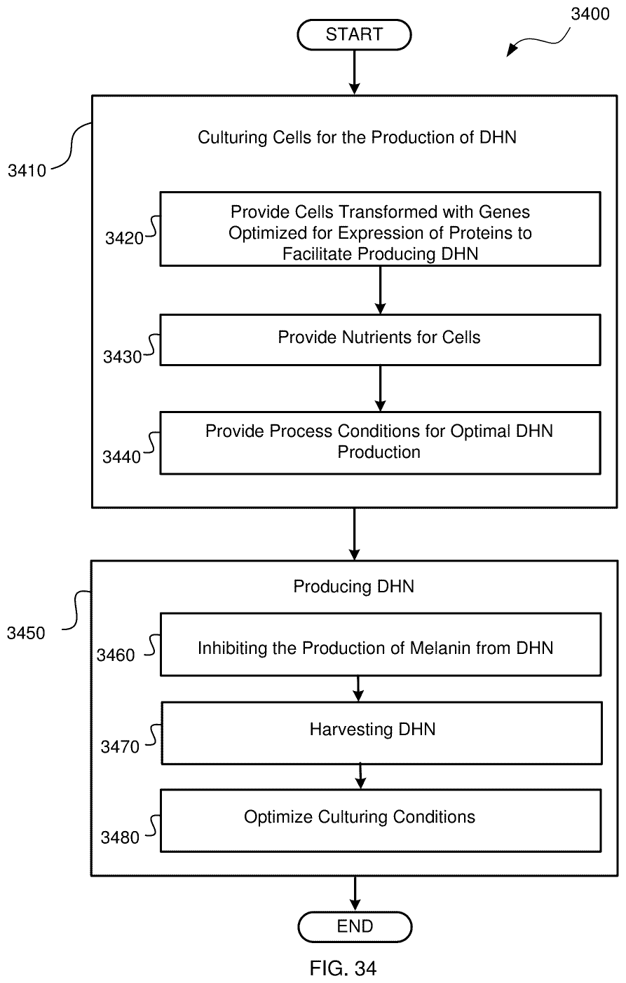

Turning now to FIG. 34, a method 3400 of producing 1,8-dihydroxy naphthalene (DHN) is shown. The method 3400 includes culturing 3410 cells under suitable culture conditions for the production of DHN. In one embodiment, the cells include a heterologous nucleic acid encoding a polyketide synthase polypeptide and one or more heterologous nucleic acids encoding one or more proteins used in the production of DHN. The method 3400 further includes producing DHN 3450. The cells may be any of the cells described herein throughout.

In one embodiment, the cells include heterologous nucleic acids encoding one or more of 1,3,8-trihydroxynapthalene (T3HN) reductase, 1,3,6,8-tetrahydroxynapthalene (T4HN) reductase, scytalone dehydratase, bacterial DHN (dihydroxynaphthalene) synthase A, and bacterial DHN (dihydroxynaphthalene) synthase B. The nucleic acids encoding a polyketide synthase polypeptide may include one or more of PKS18, soceCHS1, RpoA, and PKSwd. In one embodiment, one or more nucleic acids encoding one or more proteins used in the production of DHN includes PKS18 and either i) Brn1, Brn2, and Scd1 or ii) BdsA and BdsB.

The method 3400 includes providing 3420 cells of the kind described herein transformed with genes optimized for expression of proteins to facilitate producing DHN. In one embodiment, the cells include one or more of a nucleotide sequence of a polyketide synthase gene codon-optimized for expression in E. coli, a nucleotide sequence of a polyketide synthase gene codon-optimized for expression in P. Putida, a nucleotide sequence of a 1,3,8-trihydroxynapthalene (T3HN) reductase gene codon-optimized for expression in E. coli, a nucleotide sequence of a 1,3,8-trihydroxynapthalene (T3HN) reductase gene codon-optimized for expression in P. putida, nucleotide sequence of a 1,3,6,8-tetrahydroxynapthalene (T4HN) reductase gene codon-optimized for expression in E. coli, a nucleotide sequence of a 1,3,6,8-tetrahydroxynapthalene (T4HN) reductase gene codon-optimized for expression in P. putida, a nucleotide sequence of a scytalone dehydratase gene codon-optimized for expression in E. coli, a nucleotide sequence of a scytalone dehydratase gene codon-optimized for expression in P. putida, a nucleotide sequence of a bacterial DHN (dihydroxynaphthalene) synthase A gene codon-optimized for expression in E. coli, a nucleotide sequence of a bacterial DHN (dihydroxynaphthalene) synthase A gene codon-optimized for expression in P. putida, a nucleotide sequence of a bacterial DHN (dihydroxynaphthalene) synthase B gene codon-optimized for expression in E. coli, and a nucleotide sequence of a bacterial DHN (dihydroxynaphthalene) synthase B gene codon-optimized for expression in P. putida.

The method 3400 may include at least one cell chose from gram-positive bacterial cells, gram-negative bacterial cells, fungal cells, filamentous fungal cells, algal cells and yeast cells. The method 3400 may include one or more of the host cells described herein throughout. The method 3400 includes providing nutrients 3430 for the cells. The nutrients may include any of the nutrients described herein throughout. In one embodiment, culturing the cells 3410, and/or providing nutrients 3430 for the cells includes providing a feedstock for the host cells. The feedstock may include any of the feedstocks described herein, including without limitation any of the sugar feedstocks. In one embodiment the cells may be provided with a sugar feedstock derived from biomass.

In one embodiment, the method 3400 includes providing the process and/or culture conditions described herein throughout. In one embodiment, the conditions may include less than about 20%, 15%, 10%, 5%, or 1% oxygen.

The method 3400 includes producing DHN 3450. In one embodiment, producing DHN 3450 includes inhibiting the production of melanin from DHN 3460. This may be done using one or more melanogenesis inhibition compounds of the type described herein throughout. In one embodiment, inhibiting the production of melanin 3460 includes inhibiting expression of p-diphenol oxidase enzymes in the cells. Inhibiting melanogenesis of DHN may include removing a gene that can express p-diphenol oxidase in the cells. In one embodiment, one or more melanogenesis inhibition compounds include an antifungal compound.

Producing DHN 3450 may including harvesting the DHN 3470 from the host cell and/or culture media using methods described herein and ways known in the art. After harvesting the DHN, the yield can be measured and the culture conditions and/or processing conditions can be optimized to increase the DHN yield. In one embodiment, the methods and processes described herein can be used for the production of DHN salts.

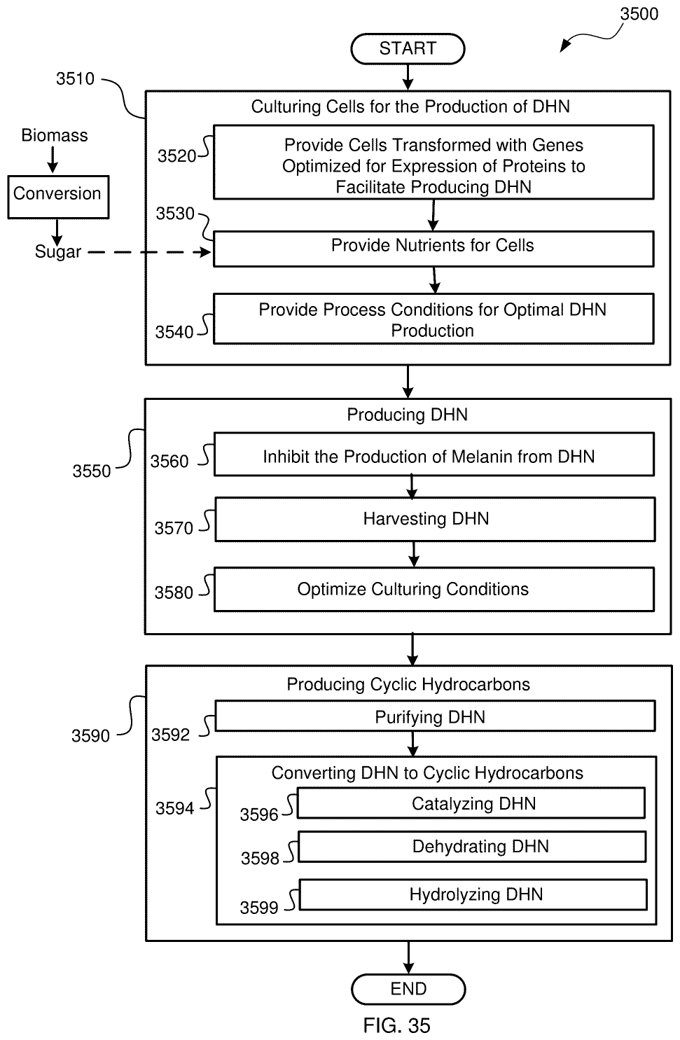

Turning now to FIG. 35, a method 3500 for producing cyclic hydrocarbons, includes culturing cells 3510 under suitable culture conditions for the production of DHN, producing DHN 3550, and converting the DHN 3590 into one or more cyclic hydrocarbons. The steps of culturing cells 3510 and producing DHN may be the same as described above, including the makeup of the cells. In one embodiment, producing cyclic hydrocarbons by converting DHN into one or more cyclic hydrocarbons Includes purifying 3592 the harvested DHN. The purified hydrocarbons may include more than about 25% decalin and/or tetralin. Producing cyclic hydrocarbons 3590 may also include at least one of catalyzing DHN 3596, dehydrating DHN 3598, and hydrolyzing DHN 3599. Converting DHN into one or more cyclic hydrocarbons may include removing two hydroxyl groups from a DHN molecule.

The method 3500 may include biosynthesis of DHN and related compounds followed by conversion to cyclic hydrocarbons, including without limitation, decalin, tetralin, bicyclic alkanes, cyclic alkanes, cyclooctanes, derivatives thereof, and the like, including without limitation methyl tetralin. In one embodiment, conversion of DHN into cyclic hydrocarbons includes at least one of the operations of (1) dehydration (removal of water, typically involving removal of a hydroxyl group from one carbon and a hydrogen from an adjacent carbon); (2) deoxygenation such as removing the oxygen of a hydroxyl group, effectively replacing the hydroxyl group with hydrogen; (3) hydrogenation (adding hydrogen to a carbon, thereby increasing the saturation of a molecule); and (4) hydrodeoxygenation (HDO), a process whereby oxygen is removed as water by adding hydrogen while retaining the carbon molecular architecture. In some embodiments, two or more of these operations may be carried out. When a plurality of such operations are involved, they may be done sequentially or simultaneously in continuous or batch operations.

The conversion of DHN into cyclic hydrocarbons may include using a catalyst including at least one of nickel, molybdenum, iron, vanadium, platinum, and palladium. In one embodiment, the catalyst may be from one or more of metals in the platinum group, noble metals, and transition elements. In one embodiment, the catalyst includes a metal ion that includes one or more of nickel, cobalt, copper, titanium, zirconium, chromium, manganese, zinc, niobium, ruthenium, iridium, scandium, yttrium, tungsten, osmium, gold, molybdenum, iron, vanadium, platinum, rhodium, rhenium, and palladium. The catalyst may take the form of carbides, nitrates, oxides, of the foregoing.

Dehydration of DHN may be done by ways known in the art. In one embodiment, dehydration may be carried out using the system described in “Dehydration of alcohols catalyzed by heteropolyacids supported on silica,” Journal of Chemical Research (S) (2001): 508-510, with the pertinent parts thereof being incorporated herein by reference.

Deoxygenation may be carried out using Barton-McCombie deoxygenation, the Markó-Lam deoxygenation, Bouveault-Blanc reduction, or other deoxygenation reactions. Alternatively, deoxygenation may be facilitated in some embodiments by first oxidizing an alcohol or ketone to a carboxylic acid and then removing the acid group. Such oxidation may be carried out, for example, by treating the compound with a Jones reagent, including without limitation, chromium trioxide (chromium(VI)) in aqueous sulfuric acid and acetone, pyridinium chlorochromate (PCC) and pyridium dichromate (PDC). The carboxylic acid group can then be removed by decarboxylation, such as by use of copper salt in quinoline or heating its silver salt, or by other means for reducing oxidized aromatics such as passing the compound over red-hot zinc dust, and the like.

A molecule of DHN or related compounds will generally need more than one oxygen group to be removed and more than one hydrogen moiety to be added, and these may occur in various steps. In one embodiment, this may be accomplished by a deoxygenation step followed by one or more hydrogenation steps, followed again by deoxygenation, and optionally further hydrogenation. Such operations can be carried out in any feasible order and in any phase involving any suitable catalyst or a plurality of catalysts and catalytic support types.

Dehydration steps or removal of hydroxyl groups may take place on one or more of the following molecules: (1) hydroxynaphthalenes, such as any monohydroxynapthalene, any dihidroxynapthalene, any trihydroxynapthelene (T₃HN), or any tetrahydroxynaphthalene (T₄HN), etc.; (2) hydrogenated hydroxynapthalenes such as 1,2,3,4-tetrahydro-1-naphthol or any hydrogenated naphthol, (3) hydrogenated napthalenes such as tetralin or its oxygenated derivatives (alcohol, ketones, etc.) such as scytalone or vermelone; (4) various oxygenated naphthalene derivatives such as flaviolin, naphthoquinones such as mompain, and the like.

Additional steps may include conversion of carbonyl groups (e.g., aldehydes, ketonea, carboxylic acids, enones, and carboxylate esters) to alcohols (e.g., using ruthenium catalysts on supports such as carbon, silica, ceria, or alumina, optionally in the presence of some water. The alcohols may then be deoxygenated or dehydrated.

One or more dehydration steps may be carried out in any of the following processes:
Elimination of hydroxyl groups using acid and heat, such as heated H2504 or heated phosphoric acid (e.g., 75% to 90% phosphoric acid in water, heated to 150° C. to 200° C. or from 140° C. to 185° C.).
Catalytic dehydration using noble metals such as Pt(111) or other previously mentioned catalysts in the presence of hydrogen or hydrogen donors.
Conversion of hydroxyl groups into good leaving groups using reaction with phosphorus tribromide (PBr3) and thionyl chloride (SOCl2).
Use of gallium(III) oxide and co-precipitated Ga(III)/M(II) oxides (M=Mg, Ca, and Sr) at elevated temperature (e.g., 300-460° C.)

One or more hydrogenation steps may be carried out using any of the following processes:

1. Use of catalysts such as palladium, platinum, rhodium, rhenium, ruthenium, cobalt, nickel, molybdenum, vanadium, and combinations thereof, oxides thereof, salts thereof, etc., and combination thereof such as Ni—Mo, Ni—W, Co—Mo, Va-Ni, Va-Mo, etc., with various supports such as Al₂O₃, SiO₂, TiO, HY, HZSM-5, activated carbon (e.g., a catalyst of p-phase Mo₂C), Al₂O₃/SiO₂, Al₂O₃/TiO₂, ceria, etc. For example, a pressurized stainless steel reactor may be used in which air is placed with nitrogen and then hydrogen is introduced, replacing all or a portion of the nitrogen, and bringing the hydrogen to a suitable pressure such as 0.4-4.0 MPa, and heating the reactor so that the reaction temperature reaches 30-300° C. or 30 to 200° C. to carry out catalytic hydrogenation. Suitable agitators and catalyst carriers may be selected such as zeolite, alumina, titanium dioxide, silica, carbon, or combinations thereof. In one aspect, hydrogenation may occur at a hydrogen pressure of 0.1 MPa or greater, such as 0.5 to 10 MPa, 0.5 to 5 MPa, or 1 to 7 MPa, and may be at temperature of 200° C. to 430° C. or 250° C. to 400° C. such as from 300° C. to 380° C. The catalytic system may comprise a combination of AL₂O₃, cobalt oxide, and molybdenum trioxide. In one aspect, a rhodium catalyst supported on carbon or other support materials may be used, either in supercritical carbon dioxide or in a liquid solvent at temperatures above 31° C. such as from 40° C. to 200° C. or from 35° C. to 150° C. In another aspect, palladium and/or platinum supported on carbon or alumina or other support materials us used at an elevated temperature such as from 200° C. to 800° C. or from 250° C. to 600° C.

2. Catalysis using a metal-organic framework (MOF) such as CuPd@ZIF-8 composite, featuring a cubic CuPd core and a porous ZIF-8 shell, as described in Luyan Li et al., “Accelerating Chemo- and Regioselective Hydrogenation of Alkynes over Bimetallic Nanoparticles in a Metal-Organic Framework,” ACS Catalysis 10/14 (2020): 7753-7762; https://doi.org/10.1021/acscatal.0c00177 and https://pubs.acs.org/doi/10.1021/acscatal.0c00177. NH3BH3 was used as the hydrogen source under visible-light irradiation.

3. Conversion over alumina (e.g., strongly acidic alumina or weakly acidic alumina) in the presence of excess methanol or other solvents at a suitable temperature such as from 150 to 350° C. In one embodiment, γ-Al₂O₃ is used as a support (for example, that produced by Aluminum Corporation of China Limited) and may have a saturated water absorption from 20% to 50%, such as from 30% to 45% or from 25% to 34%. In a related aspect, Al₂O₃ supports may be obtained through co-precipitation method and the sol-gel method, as described by Xiaoping Su et al., “Selective catalytic hydrogenation of naphthalene to tetralin over a Ni—Mo/Al₂O₃ catalyst,” Chinese Journal of Chemical Engineering, 28/10 (October 2020): 2566-2576.

4. Conversion using nickel-based catalysts, such as Ni—Mo, Ni—W and Ni—Mo—W catalysts prepared through hydrothermal synthesis. See, for example, N. Shen et al., “Hydrogenation of naphthalene to decalin on sulfurized massive Ni—Mo catalysts,” Petrochemical Technology (Shiyou Huagong), 44/7 (July 2015): 846-851.

5. Conversion using a Ni-Al₂O₃ catalyst without calcination, or a catalyst comprising NiO and from 10% to 80%, or from 10% to 40% MoO₃/Al₂O₃ catalyst which may be pre-sulphided and/or pre-reduced.

6. Iron-based catalysts such as Fe—Mo.

7. Other nickel-based catalysts include Ni nano-clusters supported on MFI nano-sheets and Ni—Al intermetallic compound catalysts on any support, optionally with a portion of the metal in the form of nanoparticles.

8. Metal carbide catalysts such as molybdenum carbide (β-Mo₂C) catalysts, optionally promoted by K₂CO₃ and/or Ni, supported on substrates that may include any form of carbon such as carbon modified SBA-15, active carbon (AC), and mesoporous carbon CMK-3.

9. Catalysts employing zeolites may be used, such as MFI nanosheet and catalysts comprising ZSM-5, Zeolite Socony Mobil-5 (framework type MFI), an aluminosilicate zeolite belonging to the pentasil family of zeolites. Pentasil (MFI) structures from Clariant include: CZP 27, CZP 30, CZP 90, CZP 200 and CZP 800. In one aspect, hierarchical MFI nanosheets with nickel species chemically bonded on the outer surfaces may be used. Mesoporous ZSM-5 or other zeolites may be used as a support for platinum or other noble metals for hydrogenation.

10. Catalysts employing mineral fiber materials may be used, such as a knitted silica fiber capable of withstanding high temperatures. Quartz fibers, glass fibers, ceramic fibers, asbestos, etc., may be considered and provided in the form of wool, felt, thread, fabrics or knits for use as carriers. In addition, silica-based fibers, both in woven (e.g., knitted) and nonwoven form may be used. In general, a viscose fiber is prepared containing silica and optionally other minerals or metals such as alumina or ceria or titania. Then, upon calcination, a fibrous silica-based material remains that can serve as a support for catalysts (similar techniques may be applied to create mineral fibers that have relatively low silica content or are silica free, including fibers largely made from alumina, ceria, etc. The fibrous material may serve, for example, as a support material for platinum catalysts, prepared with various platinum precursors, yielding platinum, bimetallic or trimetallic combinations with metals such as palladium, nickel, cobalt, molybdenum, indium, iron, chromium, tungsten, tantalum, iridium, osmium, and any transition metal or noble metal, as well as oxides, nitrates, etc., such as platinum nitrate. Chemisorption, impregnation, evaporation from an alcohol or water solution or other methods may be used to place catalytic materials on the surface of this or other supports mentioned herein. Metal loading may be from 1% to 25%, 1% to 10%, or from 2% to 7%. Gas-phase hydrogenation of aromatic compounds such as naphthalene and its derivatives or partially saturated forms thereof may take place at 50° C. to 400° C. or from 50° C. to 300° C. or from 70° C. to 250° C. and at hydrogen partial pressures from 0.03 to 3 bar, such as from 0.05 to 1.5 bar or from 0.05 to 1 bar.

Hydrodeoxygenation may be carried out using catalysts such as rhenium, palladium, or platinum. In some embodiments, oxophilic metals such as iron, ruthenium, and cobalt-molybdenum-sulfide (CoMoS) catalysts may be successful. Acid-based supported catalysts may be used, but non-acid-based supported catalysts such as Rh/C, Pd/C, Pt/C, Pt/SiO2, Pd—Fe/C, and Ni/SiO2 may also perform well. High pressure hydrodeoxygenation (HDO) is one route. Catalytic fast pyrolysis with zeolites may also be used. Traditional hydro-desulphurization (HDS) catalysts, such as cobalt MoS₂/Al₂O₃ or metal catalysts such as Pd/C, or alternative such as Raney Nickel combined with Nafion/SiO₂.

In some catalytic operations, a “chaser” such as toluene can be added during the latter portion of a batch operation with distillation to helped extract a higher yield of the reactant.

In some cases, a partially saturated cyclic or bicyclic hydrocarbon produced from the biosynthesis process and possibly subsequent chemical reactions may be treated to add one or more hydroxyl groups, which can subsequently be removed via deoxygenation or via dehydration to yield a less saturated hydrocarbon. For example, a compound such as 1,2 dihydronapthalene can be converted in the presence of hydrogen peroxide and the enzyme peroxygenase to various naphthalene hydrates with one or more hydroxyl groups added, and the hydroxyl group(s) can then be dehydrated, leaving a more saturated ring, when desired.

Suitable catalysts and supports can be obtained from a variety of sources such as BASF Catalysts (Iselin, NJ), Riogen Inc. (Mullica Hill, NJ), Haldor Topsoe (Denmark), UOP (Des Plaines, IL), Axens (Rueil Malmaison, France), Johnson Matthey (Royston, UK), Shell Catalysts & Technologies (The Hague, Netherlands), Albemarle (Charlotte, NC), Exxonmobil Chemical (Houston, TX), and Grace Davison (Columbia, MD). “BASF” is a registered trademark of BASF SE SOCIETAS EUROPAEA. “HALDOR TOPSOE” is a registered trademark of Haldor Topsoe A/S PUBLIC LIMITED COMPANY. “UOP” is a registered trademark of UOP Inc. “AXENS” is a registered trademark of Axens SOCIÉTÉ ANONYME (SA). “SHELL” is a registered trademark of Shell Trademark Management B.V. LIMITED LIABILITY COMPANY. “ALBEMARLE” is a registered trademark of ALBEMARLE CORPORATION. EXXONMOBIL is a registered trademark of EXXON MOBIL CORPORATION. “GRACE” is a registered trademark of W. R. GRACE & CO.

In one embodiment, the cyclic hydrocarbons are bicyclic hydrocarbons suitable for use in fuels such as jet fuel. In one embodiment, a method includes converting biomass into jet fuels by converting the biomass into sugar, feeding the sugar to cells described herein throughout to produce DHN and converting DHN into one or more types of jet fuel or jet fuel components. Additional method or process steps include those described with other embodiments herein throughout.

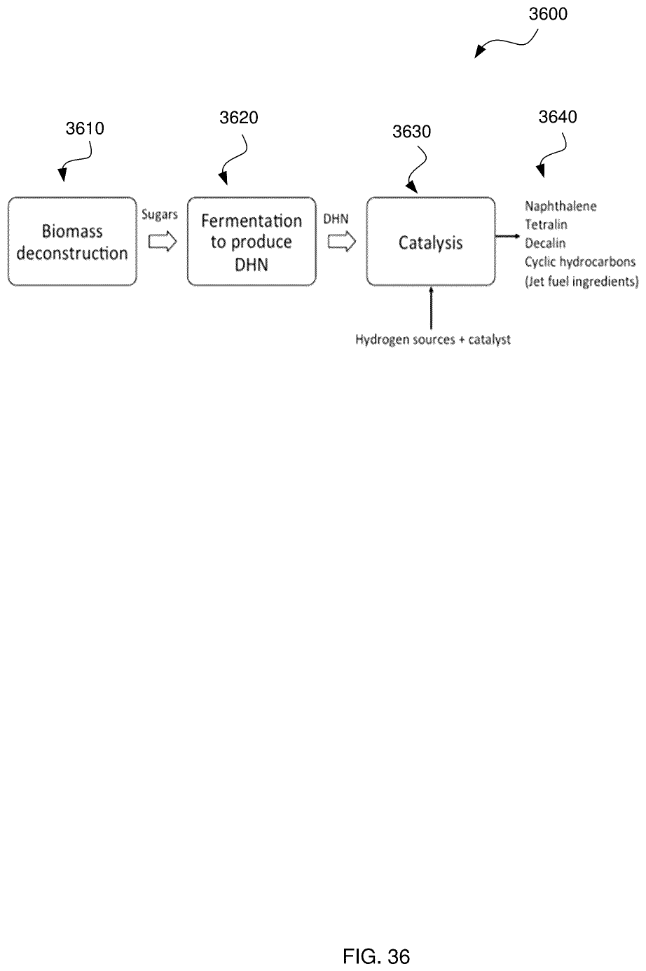

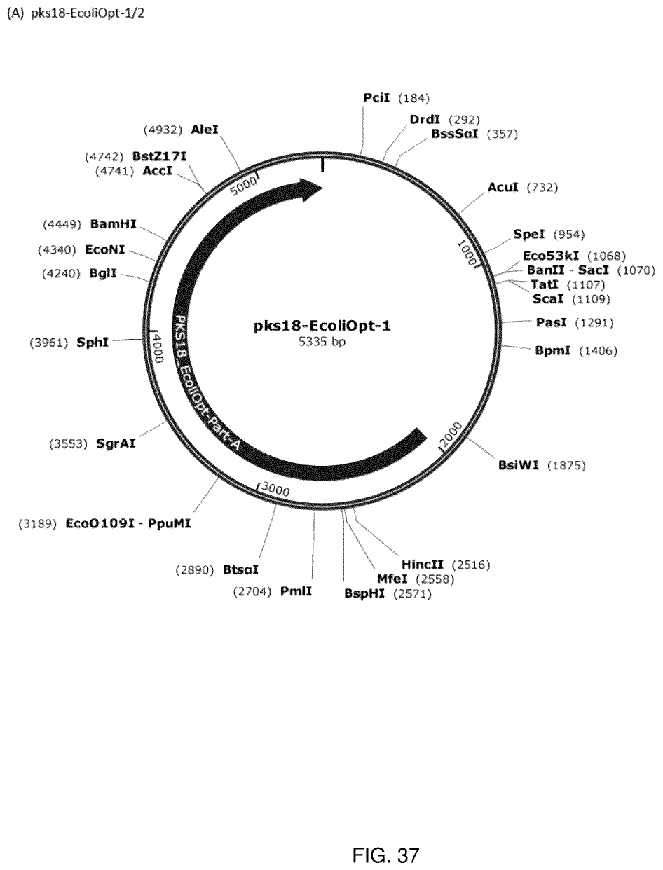

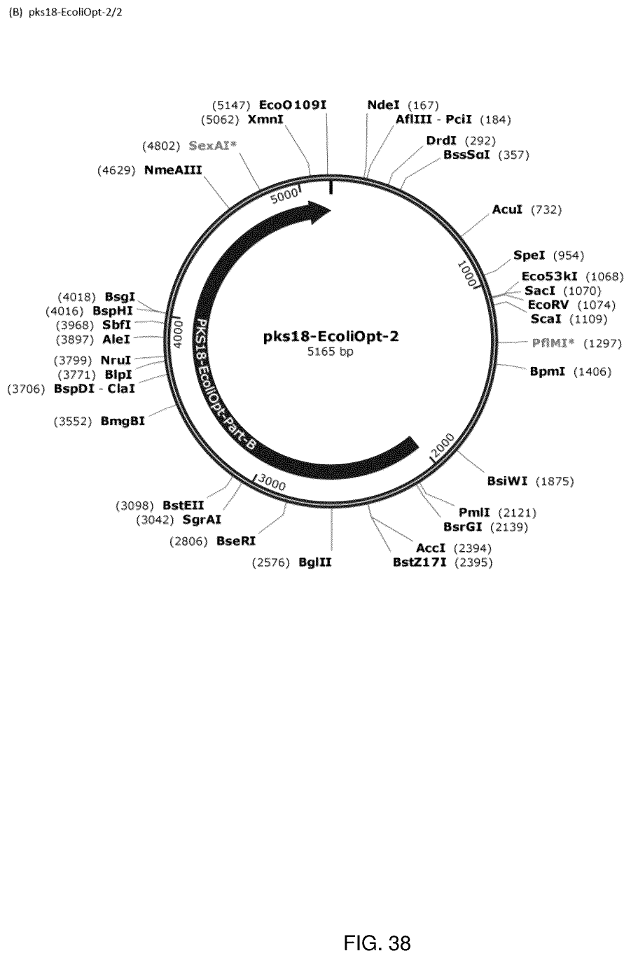

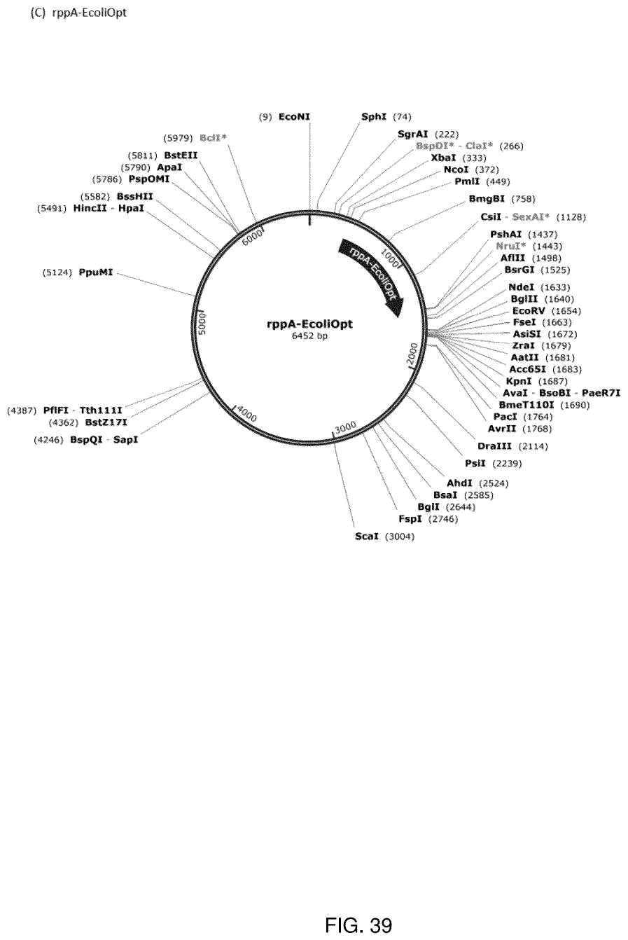

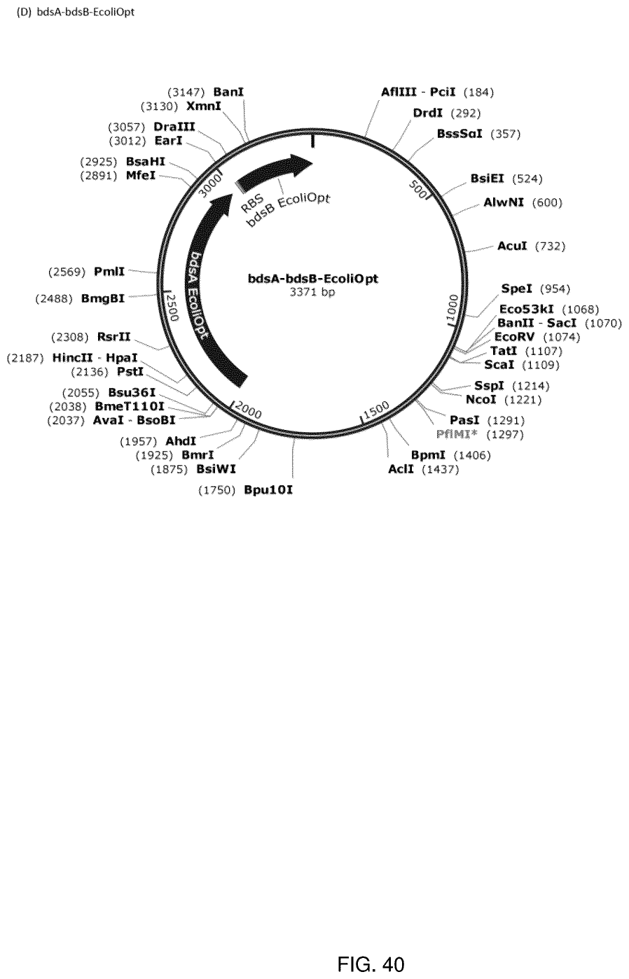

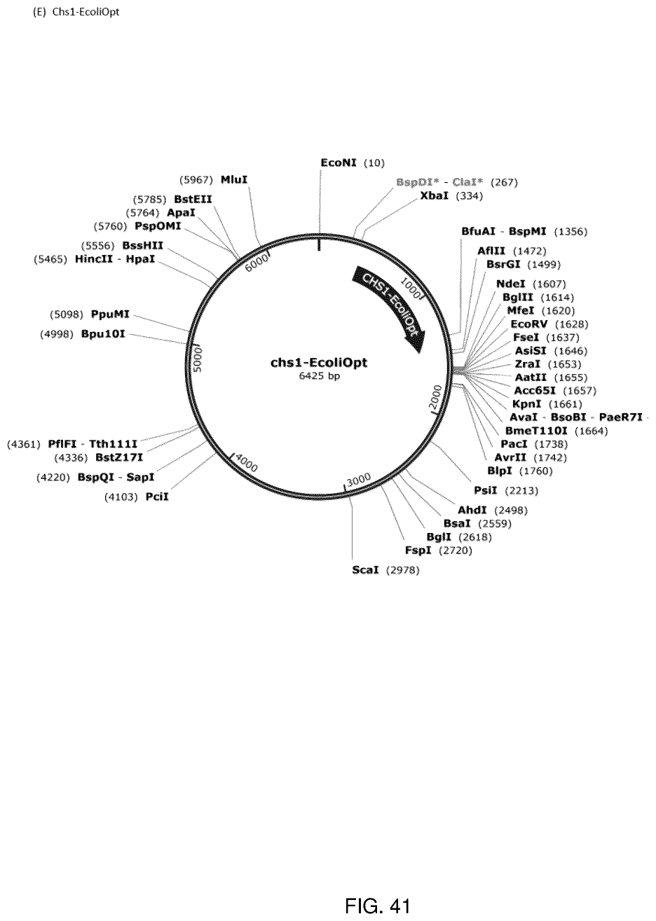

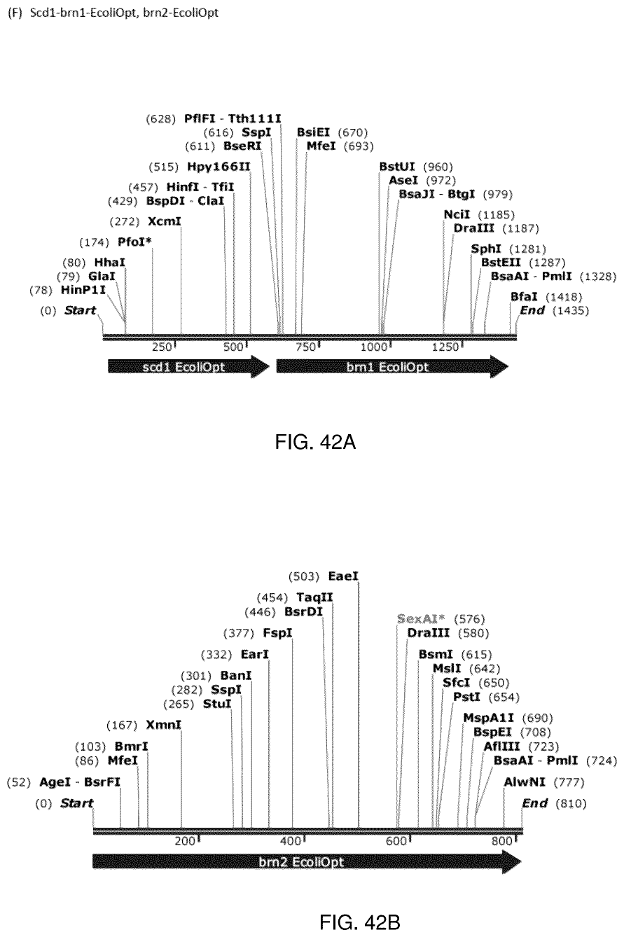

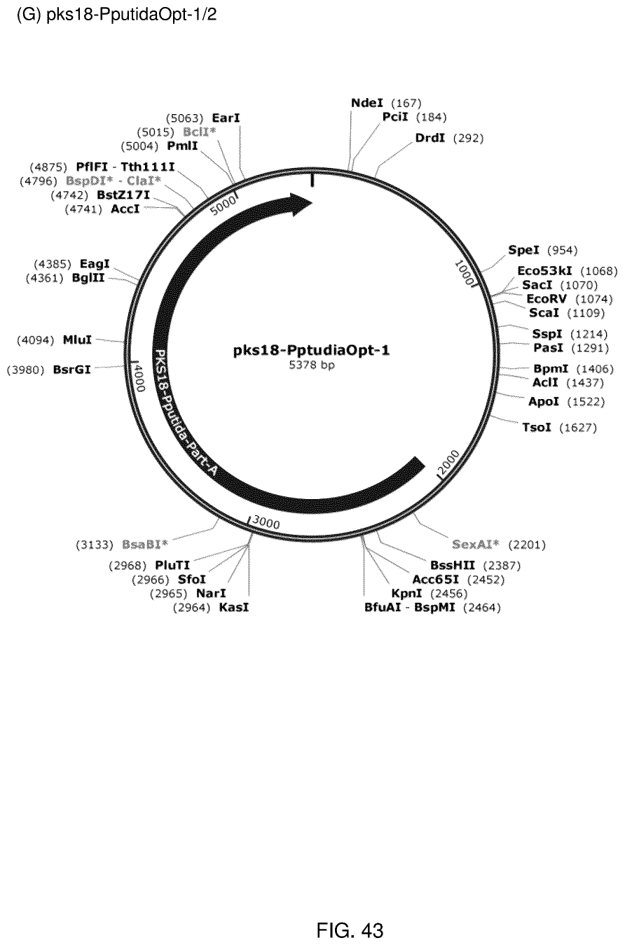

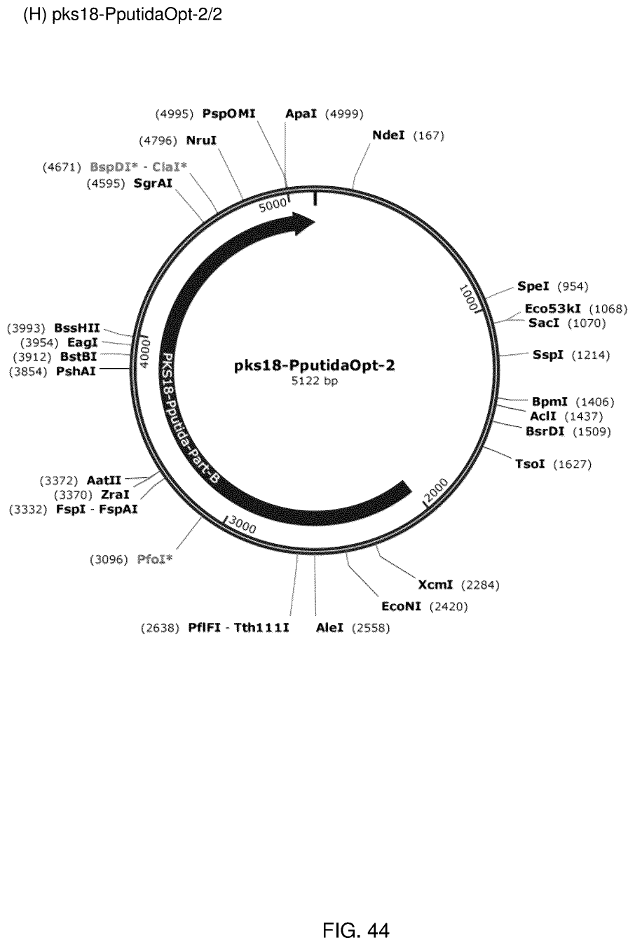

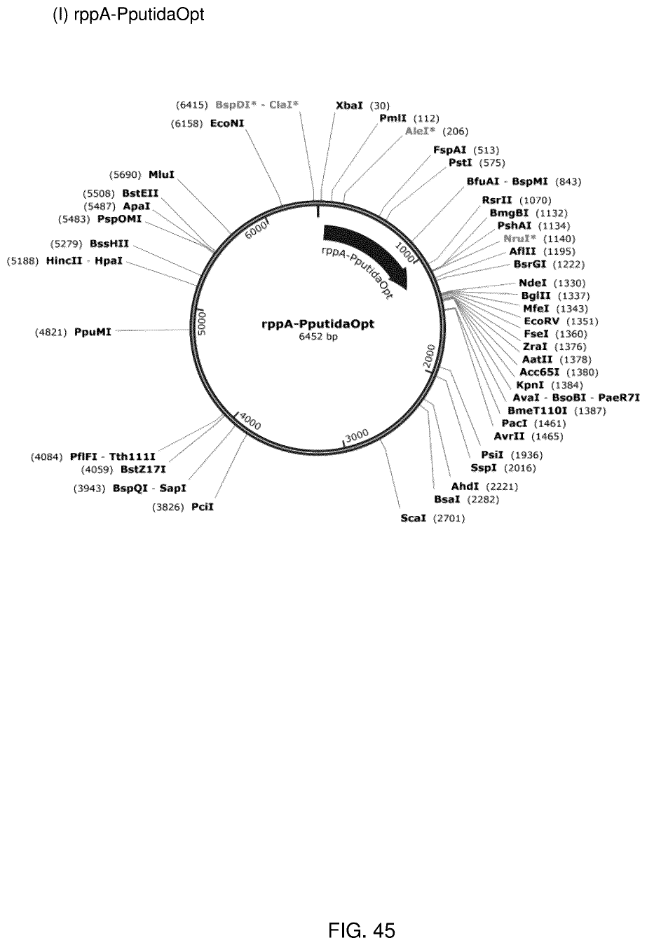

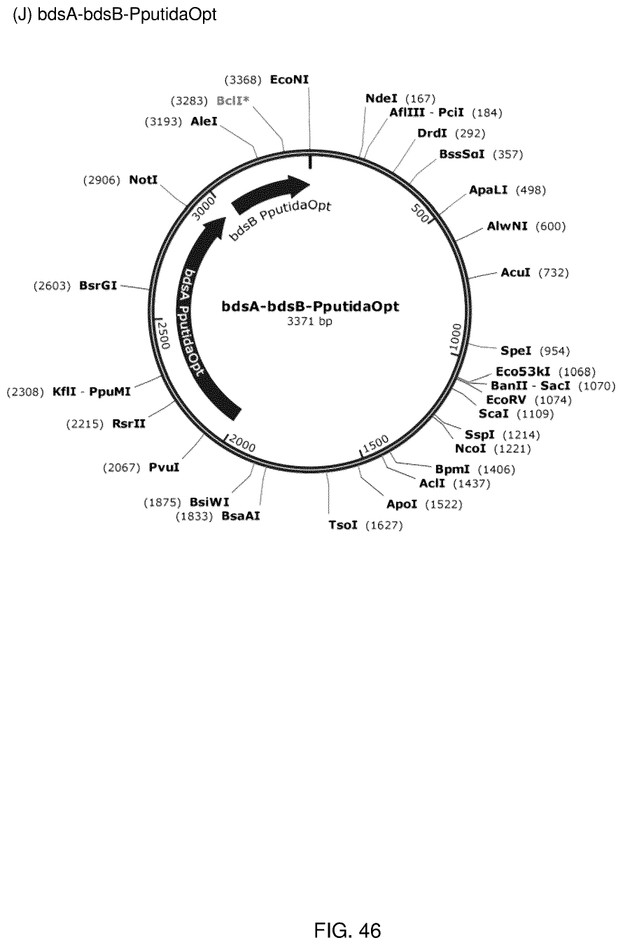

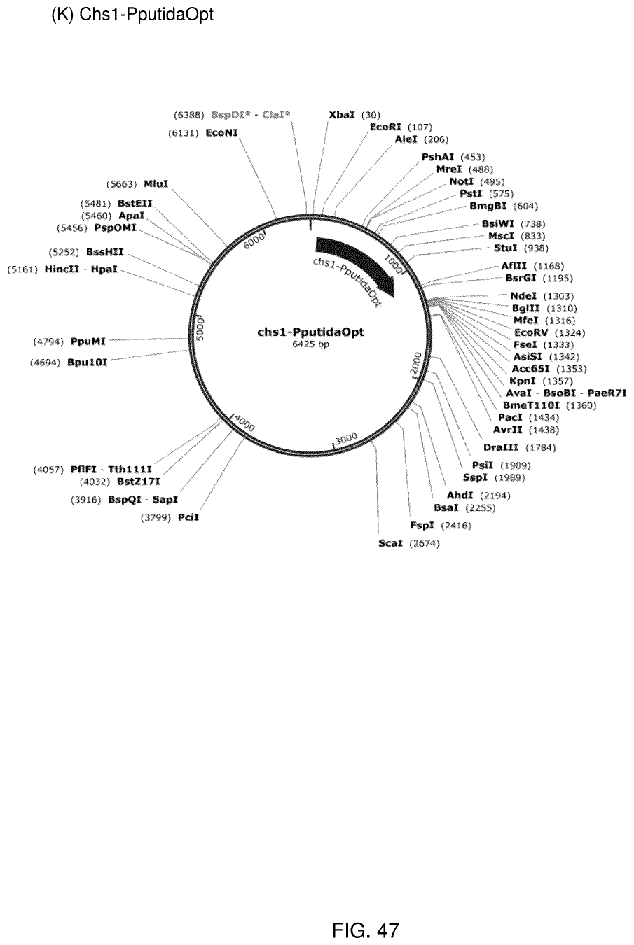

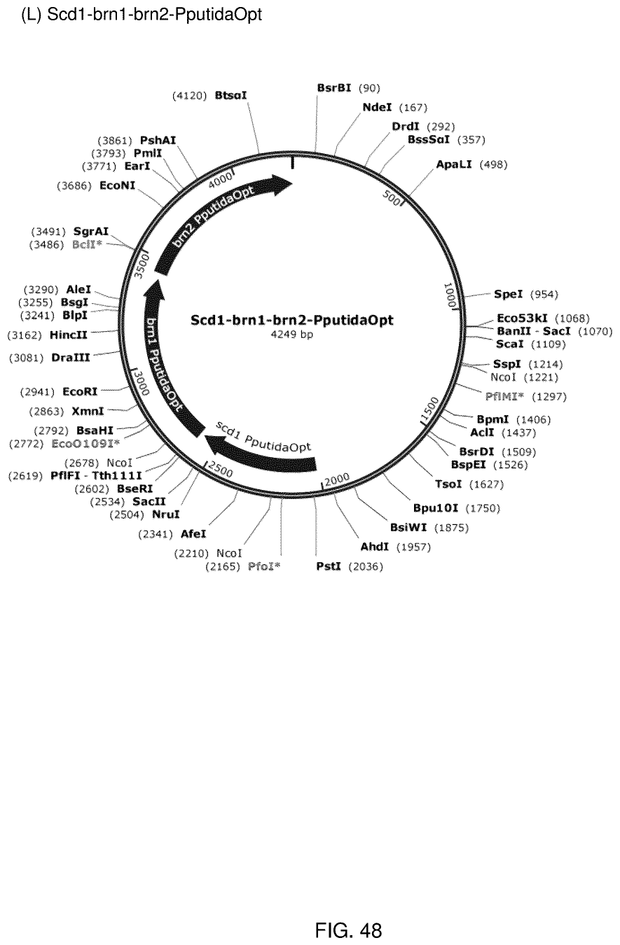

Turning now to FIG. 36, the resulting blend of cyclic hydrocarbon compounds may be suitable for jet fuel or other targeted fuel types, or may be further blended with other alkanes, cyclic alkanes, etc., to meet desired specifications for jet fuel or other targeted fuels. A method 3600 for producing fuel from biomass may include deconstructing biomass 3610 to make sugar, fermenting sugar 3620 to produce DHN, catalyzing DHN 3630 to make fuel 3640, including jet fuel and components thereof. In one embodiment, the catalysis step 3630 may include adding hydrogen and/or a catalyst. Step 3520 may include any of the cells and method or process steps described herein to produce DHN. The catalysis step 3630 may include any of the cells and method or process steps described herein to make cyclic hydrocarbons, including bicyclic hydrocarbons.

Methods of making fuel may be modified to meet the required properties of the desired fuel. For example, jet engine fuels are meant to feed the burners of aircraft turbojet and jet engines. Approved jet engine fuels must have certain properties. For example, jet engine fuel Jet A1, which is the jet engine fuel most commonly used in commercial aviation, must have a sulfur content of less than 0.30 wt. %, a content of aromatic compounds of less than 22% by volume, a flash point above 38° C., a smoke point above 25 mm and a lower freezing point of −47° C., while for Jet A the lower freezing point is −40° C.

In one aspect, a resulting blend of cyclic hydrocarbons, or in other words, the results of method 3600 may, in one embodiment, be suitable as a jet engine fuel having the following characteristics:
i) distilling range from 140 to 300° C.;
ii) cis-decalin/trans-decalin ratio greater than 0.2;
iii) aromatics content less than 22% by volume;
iv) sulfur content less than 100 ppm, and
v) lower heating value per unit volume greater than 34.65 Mj/liter.

Further, the fuel's naphthalene/decalin ratio may be less than 0.2, 0.1, or 0.05. The same may also apply to the naphthalene/trans-decalin ratio.

Preparing the converted naphthalene into a suitable fuel may comprise taking a cut from catalytic cracking distilling between 140 and 300° C., and also providing an optional hydrotreatment step and an optional dearomatization step.

Prior to mixing to form jet fuel, unreacted components such as remaining naphthalene or any undesired by produces may be removed from the reactant stream using distillation, reverse osmosis, crystallization, or any suitable separation method.

All patents and patent publications cited herein may be presumed to be incorporated by reference to the extent it is non-contradictory herewith. When a document is explicitly said to be “incorporated by reference,” it is also implied that it is incorporated by reference to the extent it is non-contradictory herewith.

Having described aspects of the invention in detail, it will be apparent that modifications and variations are possible without departing from the scope of aspects of the invention as defined in the appended claims. As various changes could be made in the above compositions, products, and methods without departing from the scope of aspects of the invention, it is intended that all matter contained in the above description shall be interpreted as illustrative and not in a limiting sense.

While the foregoing makes reference to particular illustrative embodiments, these examples should not be construed as limitations. The inventive system, methods, and products can be adapted for other uses or forms not explicitly listed above, and can be modified in numerous ways within the spirit of the present disclosure. Thus, the present invention is not limited to the disclosed embodiments, but is to be accorded the widest scope consistent with the claims below.

We claim:

1. Cells, comprising: a nucleic acid encoding a polyketide synthase polypeptide and one or more nucleic acids encoding one or more proteins used in the production of DHN.

2. The cells of claim 1, wherein the proteins comprise proteins in a DHN melanin pathway.

3. The cells of claim 2, wherein the DHN melanin pathway comprises the DHN melanin pathway found in Cochliobolus heterostrophus.

4. The cells of claim 2, wherein the DHN melanin pathway comprises the DHN melanin pathway found in Sorangium cellulosum.

5. The cells of claim 2, wherein the DHN melanin pathway comprises the DHN melanin pathway found in Streptomyces griseus.

6. The cells of claim 1, wherein the nucleic acid encoding a polyketide synthase polypeptide is a heterologous nucleic acid.

7. The cells of claim 1, wherein the polyketide synthase is a type I polyketide synthase.

8. The cells of claim 1, wherein the polyketide synthase is a type III polyketide synthase.

9. The cells of claim 1, wherein the cells further comprise one or more nucleic acids encoding the polypeptides of an entire DHN melanin pathway

10. The cells of claim 1, wherein the polyketide synthase polypeptide is a fungal polyketide synthase polypeptide.

11. The cells of claim 1, wherein the polyketide synthase polypeptide is a bacterial polyketide synthase polypeptide.

12. The cells of claim 1, further comprising nucleic acids encoding one or more of 1,3,8-trihydroxynapthalene (T3HN) reductase, 1,3,6,8-tetrahydroxynapthalene (T4HN) reductase, scytalone dehydratase, bacterial DHN (dihydroxynaphthalene) synthase A, and bacterial DHN (dihydroxynaphthalene) synthase B.

13. The cells of claim 1, wherein the nucleic acids encoding a polyketide synthase polypeptide comprise one or more of PKS18, soceCHS1, RppA, and PKSwd.

14. The cells of claim 1, wherein the one or more nucleic acids encoding one or more proteins used in the production of DHN comprises PKS18 and either i) Brn1, Brn2, and Scd1 or ii) BdsA and BdsB.

15. The cells of claim 1, wherein the cells further comprise one or more of a nucleotide sequence of a polyketide synthase gene codon-optimized for expression in E. coli, a nucleotide sequence of a polyketide synthase gene codon-optimized for expression in P. putida, a nucleotide sequence of a 1,3,8-trihydroxynapthalene (T3HN) reductase gene codon-optimized for expression in E. coli, a nucleotide sequence of a 1,3,8-trihydroxynapthalene (T3HN) reductase gene codon-optimized for expression in P. putida, nucleotide sequence of a 1,3,6,8-tetrahydroxynapthalene (T4HN) reductase gene codon-optimized for expression in E. coli, a nucleotide sequence of a 1,3,6,8-tetrahydroxynapthalene (T4HN) reductase gene codon-optimized for expression in P. putida, a nucleotide sequence of a scytalone dehydratase gene codon-optimized for expression in E. coli, a nucleotide sequence of a scytalone dehydratase gene codon-optimized for expression in P. putida, a nucleotide sequence of a bacterial DHN (dihydroxynaphthalene) synthase A gene codon-optimized for expression in E. coli, a nucleotide sequence of a bacterial DHN (dihydroxynaphthalene) synthase A gene codon-optimized for expression in P. putida, a nucleotide sequence of a bacterial DHN (dihydroxynaphthalene) synthase B gene codon-optimized for expression in E. coli, and a nucleotide sequence of a bacterial DHN (dihydroxynaphthalene) synthase B gene codon-optimized for expression in P. putida.

16. The cells of claim 1, further comprising at least one cell chose from gram-positive bacterial cells, gram-negative bacterial cells, fungal cells, filamentous fungal cells, algal cells and yeast cells.

17. The cells of claim 16, wherein the cells comprise one or more of Escherichia coli, Pseudomonas putida, Pantoae citrea, Bacillus subtilis, Bacillus licheniformis, Bacillus lentus, Bacillus brevis, Bacillus stearothermophilus, Bacillus alkalophilus, Bacillus amyloliquefaciens, Bacillus clausii, Bacillus halodurans, Bacillus megaterium, Bacillus coagulans, Bacillus circulans, Bacillus lautus, Bacillus thuringiensis, Streptomyces lividans, Streptomyces coelicolor, Streptomyces griseus, Pseudomonas sp., and Pseudomonas alcaligenes cells.

18. The cells of claim 1, further comprising one or more melanogenesis inhibition compounds.

19. A method of producing 1,8-dihydroxy naphthalene (DHN), the method comprising: (a) culturing cells under suitable culture conditions for the production of DHN, the cells comprising (i) a nucleic acid encoding a polyketide synthase polypeptide and one or more nucleic acids encoding one or more proteins used in the production of DHN; and (b) producing DHN.

20. The method of claim 19, wherein the cells further comprise nucleic acids encoding one or more of 1,3,8-trihydroxynapthalene (T3HN) reductase, 1,3,6,8-tetrahydroxynapthalene (T4HN) reductase, scytalone dehydratase, bacterial DHN (dihydroxynaphthalene) synthase A, and bacterial DHN (dihydroxynaphthalene) synthase B.

21. The method of claim 19, wherein the nucleic acids encoding a polyketide synthase polypeptide comprise one or more of PKS18, soceCHS1, RppA, and PKSwd.

22. The method of claim 19, wherein the one or more nucleic acids encoding one or more proteins used in the production of DHN comprises PKS18 and either i) Brn1, Brn2, and Scd1 or ii) BdsA and BdsB.

23. The method of claim 19, wherein the cells further comprise one or more of a nucleotide sequence of a polyketide synthase gene codon-optimized for expression in E. coli, a nucleotide sequence of a polyketide synthase gene codon-optimized for expression in P. putida, a nucleotide sequence of a 1,3,8-trihydroxynapthalene (T3HN) reductase gene codon-optimized for expression in E. coli, a nucleotide sequence of a 1,3,8-trihydroxynapthalene (T3HN) reductase gene codon-optimized for expression in P. putida, nucleotide sequence of a 1,3,6,8-tetrahydroxynapthalene (T4HN) reductase gene codon-optimized for expression in E. coli, a nucleotide sequence of a 1,3,6,8-tetrahydroxynapthalene (T4HN) reductase gene codon-optimized for expression in P. putida, a nucleotide sequence of a scytalone dehydratase gene codon-optimized for expression in E. coli, a nucleotide sequence of a scytalone dehydratase gene codon-optimized for expression in P. putida, a nucleotide sequence of a bacterial DHN (dihydroxynaphthalene) synthase A gene codon-optimized for expression in E. coli, a nucleotide sequence of a bacterial DHN (dihydroxynaphthalene) synthase A gene codon-optimized for expression in P. putida, a nucleotide sequence of a bacterial DHN (dihydroxynaphthalene) synthase B gene codon-optimized for expression in E. coli, and a nucleotide sequence of a bacterial DHN (dihydroxynaphthalene) synthase B gene codon-optimized for expression in P. putida.

24. The method of claim 19, further comprising at least one cell chose from gram-positive bacterial cells, gram-negative bacterial cells, fungal cells, filamentous fungal cells, algal cells and yeast cells.

25. The method of claim 19, wherein culturing the cells comprises providing a sugar feedstock for the host cells.

26. The method of claim 25, wherein the sugar feedstock is derived from biomass.

27. The method of claim 25, wherein producing DHN comprises inhibiting melanogenesis of DHN.

28. The method of claim 27, wherein inhibiting melanogenesis of DHN comprises inhibiting expression of p-diphenol oxidase enzymes in the cells.

29. The method of claim 27, wherein inhibiting melanogenesis of DHN comprises removing a gene that can express p-diphenol oxidase in the cells.

30. The method of claim of 19, wherein at least a portion of the method is carried out in atmosphere having less than 10% oxygen.

31. A method for producing cyclic hydrocarbons, comprising: (a) culturing cells under suitable culture conditions for the production of DHN; (b) producing DHN; (c) harvesting the DHN; and removing hydroxyl groups from a DHN molecule to form a cyclic hydrocarbon.

32. The method of claim 31, wherein the cells comprise (i) a nucleic acid encoding a polyketide synthase polypeptide and one or more nucleic acids encoding one or more proteins used in the production of DHN.

33. The method of claim 32, further comprising nucleic acids encoding one or more of 1,3,8-trihydroxynapthalene (T3HN) reductase, 1,3,6,8-tetrahydroxynapthalene (T4HN) reductase, scytalone dehydratase, bacterial DHN (dihydroxynaphthalene) synthase A, and bacterial DHN (dihydroxynaphthalene) synthase B.

34. The method of claim 33, wherein the cells further comprise one or more of a nucleotide sequence of a polyketide synthase gene codon-optimized for expression in E. coli, a nucleotide sequence of a polyketide synthase gene codon-optimized for expression in P. putida, a nucleotide sequence of a 1,3,8-trihydroxynapthalene (T3HN) reductase gene codon-optimized for expression in E. coli, a nucleotide sequence of a 1,3,8-trihydroxynapthalene (T3HN) reductase gene codon-optimized for expression in P. putida, nucleotide sequence of a 1,3,6,8-tetrahydroxynapthalene (T4HN) reductase gene codon-optimized for expression in E. coli, a nucleotide sequence of a 1,3,6,8-tetrahydroxynapthalene (T4HN) reductase gene codon-optimized for expression in P. putida, a nucleotide sequence of a scytalone dehydratase gene codon-optimized for expression in E. coli, a nucleotide sequence of a scytalone dehydratase gene codon-optimized for expression in P. putida, a nucleotide sequence of a bacterial DHN (dihydroxynaphthalene) synthase A gene codon-optimized for expression in E. coli, a nucleotide sequence of a bacterial DHN (dihydroxynaphthalene) synthase A gene codon-optimized for expression in P. putida, a nucleotide sequence of a bacterial DHN (dihydroxynaphthalene) synthase B gene codon-optimized for expression in E. coli, and a nucleotide sequence of a bacterial DHN (dihydroxynaphthalene) synthase B gene codon-optimized for expression in P. putida.

35. The method of claim 31, wherein the cells further comprise at least one cell chosen from gram-positive bacterial cells, gram-negative bacterial cells, fungal cells, filamentous fungal cells, algal cells and yeast cells.

36. The method of claim 31, wherein removing hydroxyl groups from the DHN to form a cyclic hydrocarbon comprises at least one of deoxygenation, hydration and hydrogenation.

37. The method of claim 31, wherein removing hydroxyl groups from the DHN to form a cyclic hydrocarbon comprises using a catalyst comprising a metal ion comprising one or more of nickel, cobalt, copper, titanium, zirconium, chromium, manganese, zinc, niobium, ruthenium, iridium, scandium, yttrium, tungsten, osmium, gold, molybdenum, iron, vanadium, platinum, rhodium, rhenium, and palladium.

38. The method of claim 31, wherein at least a portion of the method is carried out in atmosphere having less than 10% oxygen.

39. A method for producing jet fuel from biomass, comprising: (a) deconstructing biomass to make sugar, fermenting sugar with proteins used to convert sugar into DHN, catalyzing DHN to make jet fuel components.
